(19) United States
(12) Patent Application Publication
(10) Pub. No.: US 2021/0275480 A1
(43) Pub. Date: Sep. 9, 2021

(54) COMPOSITIONS AND METHODS FOR THE REDUCTION OR TREATMENT OF FIBROSIS

(71) Applicant: AXCELLA HEALTH INC., Cambridge, MA (US)

(72) Inventors: Michael Hamill, Wellesley, MA (US); Raffi Afeyan, Cambridge, MA (US); Chung-Wei Lee, Waban, MA (US); Harry Luithardt, Cambridge, MA (US); Luke S. Hamm, Boston, MA (US); Svetlana Marukian, Watertown, MA (US); Nadine Daou, Medford, MA (US)

(21) Appl. No.: 17/254,089

(22) PCT Filed: Jun. 19, 2019
(86) PCT No.: PCT/US2019/038036

§ 371 Date: Dec. 18, 2020

Related U.S. Application Data
(60) Provisional application No. 62/794,154, filed on Jan. 18, 2019.
(60) Provisional application No. 62/758,174, filed on Nov. 9, 2018.
(60) Provisional application No. 62/687,718, filed on Jun. 20, 2018.

(51) Int. Cl. A61K 31/198; A61K 38/02; A61P 21/00; A23L 33/175

(52) U.S. Cl. CPC A61K 31/198; A23L 33/175; A61P 21/00; A61K 38/02

(57) ABSTRACT

This disclosure provides compositions and methods for reducing or treating fibrosis, e.g., in a subject having a fibrotic condition or disorder.

### BACKGROUND

Fibrosis is a serious health problem characterized by the development of excess fibrous connective tissue due at least in part to reparative or reactive processes, such as in response to an injury. In fibrosis, the abnormal accumulation of extracellular matrix proteins can result in scarring and thickening of the affected tissue. Fibrosis can occur in various organs including the lung, liver, heart, kidney, pancreas, skin, and brain. Various conditions and disorders are accompanied by fibrosis, such as cardiomyopathies, hypertension, arterial stiffness, chronic hepatitis C infection, Crohn's disease, adult respiratory distress syndrome, and sarcoidosis. Currently available therapies for fibrotic conditions have limited efficacy.

Given the limitations of available treatments, there is still a need for anti-fibrotic agents, e.g., dietary compositions and therapeutics that reduce fibrosis in a subject.

### SUMMARY OF THE INVENTION

Provided herein is a composition including amino acid entities that is useful for improving or reducing fibrosis in a subject, e.g., a subject with a fibrotic condition or disorder. The composition can be used in a method of reducing and/or treating (e.g., reversing, reducing, ameliorating, or preventing) fibrosis in a subject in need thereof (e.g, a human). The method can further include monitoring the subject for an improvement in one or more symptoms of fibrosis after administration of the composition including amino acid entities.

In one aspect, the invention features a method for reducing fibrosis in a subject, comprising administering to the subject in need thereof an effective amount of a composition (e.g., an Active Moiety) comprising:

a) a leucine amino acid entity,

b) a arginine amino acid entity,

c) glutamine amino acid entity; and

d) a N-acetylcysteine (NAC) entity;

thereby reducing fibrosis in the subject.

In some embodiments, the fibrosis is not liver fibrosis.

In another aspect, the invention features a method of treating a fibrotic condition or disorder in a subject in need thereof, comprising administering to the subject an effective amount of a composition (e.g., an Active Moiety) comprising:

a) a leucine amino acid entity,

b) a arginine amino acid entity,

c) glutamine amino acid entity; and

d) NAC entity;

thereby treating the fibrotic condition or disorder.

In some embodiments, the fibrotic condition or disorder is not a liver fibrotic condition or disorder.

In another aspect, the invention features a composition for use in reducing fibrosis in a subject, comprising an effective amount of a composition comprising:

a) a leucine amino acid entity,

b) an arginine amino acid entity,

c) glutamine amino acid entity; and

d) a N-acetylcysteine (NAC)-entity;

provided that:

the fibrosis is not liver fibrosis.

In another aspect, the invention features a composition for use in intreating a fibrotic condition or disorder in a subject in need thereof, comprising an effective amount of a composition comprising:

a) a leucine-amino acid entity

b) an arginine-amino acid entity,

c) glutamine-amino acid entity; and

d) NAC-entity;

provided that:

the fibrotic condition or disorder is not a liver fibrotic condition or disorder.

In some embodiments, the fibrotic condition or disorder is chosen from a lung fibrotic condition or disorder, a heart or vasculature fibrotic condition or disorder, a kidney fibrotic condition or disorder, a pancreas fibrotic condition or disorder, a skin fibrotic condition or disorder, a gastrointestinal fibrotic condition or disorder, a bone marrow or hematopoietic tissue fibrotic condition or disorder, a nervous system fibrotic condition or disorder, an eye fibrotic condition or disorder, or a combination thereof.

In some embodiments, administration of the composition (e.g., the Active Moiety) results in a reduction or inhibition of one, two, three, four, five, six, or more (e.g., all) of: (a) formation or deposition of tissue fibrosis; (b) the size, cellularity, composition, or cellular content, of a fibrotic lesion; (c) the collagen of a fibrotic lesion; (d) the collagen or hydroxyproline content, of a fibrotic lesion; (e) expression or activity of a fibrogenic protein; (f) fibrosis associated with an inflammatory response; or (g) weight loss associated with fibrosis.

In some embodiments, the method further comprises determining the level of one, two, three, four, five, six, seven, eight, nine, ten, or more (e.g., all) of the following: (a) Col1a1; (b) FGF-21; (c) hydroxyproline content; (d) IL-1β; (e) matrix metalloproteinase (MMP), e.g., MMP-13, MMP-2, MMP-9, MT1-MMP, MMP-3, or MMP-10; (f) N-terminal fragment of type III collagen (proC3); (g) PIIINP (N-Terminal Propeptide of Type III Collagen); (h) α-smooth muscle actin (aSMA); (i) TGF-β; (j) tissue inhibitor of metalloproteinase (TIMP); e.g., TIMP1 or TIMP2; or (k) Hsp47.

In some embodiments, the composition (e.g., the Active Moiety) further comprises one or both of (e) an isoleucine-amino acid entity or (f) a valine amino acid entity.

In some embodiments, the total wt. % of (a)-(d) or (a)-(f) is greater than the total wt. % of one, two, or three of other amino acid entity components, non-amino acid entity protein components (e.g., whey protein), or non-protein components in the composition (e.g., in dry form), e.g., (a)-(d) or (a)-(f) is at least: 50 wt. %, 75 wt. %, or 90 wt. % of the total wt. of one or both of amino acid entity components or total components in the composition (e.g., in dry form). In some embodiments, the comprises a combination of 18 or fewer, 15 or fewer, or 10 or fewer amino acid entities, e.g., the combination comprising at least: 42 wt. %, 75 wt. %, or 90 wt. % of the total wt. of amino acid entity components or total components in the composition (e.g., in dry form).

In some embodiments, the composition does not comprise a peptide of more than 20 amino acid residues in length (e.g., whey protein), or if a peptide of more than 20 amino acid residues in length is present, the peptide is present at less than: 10 wt. %, 1 wt. %, 0.5 wt. %, 0.1 wt. %, 0.05 wt. %, 0.01 wt. %, 0.001 wt. %, or less of the total wt. of non-amino acid entity protein components or total components of the composition (e.g., in dry form).

In some embodiments, at least one, two, three, or more (e.g., all) of methionine, tryptophan, valine, or cysteine is absent from the composition, or if present, are present at less than: 10 wt. %, 1 wt. %, 0.5 wt. %, 0.1 wt. %, 0.05 wt. %, 0.01 wt. %, 0.001 wt. %, or less, e.g., of the total wt. of total components in the composition (e.g., in dry form). In some embodiments, one, two, three, or more (e.g., all) of methionine, tryptophan, valine, or cysteine, if present, are present in free form. In some embodiments, one, two, three, or more (e.g., all) of methionine, tryptophan, valine, or cysteine, if present, are present in salt form.

In some embodiments, methionine, tryptophan, valine, or cysteine, if present, may be present in an oligopeptide, polypeptide, or protein, with the proviso that the protein is not whey, casein, lactalbumin, or any other protein used as a nutritional supplement, medical food, or similar product, whether present as intact protein or protein hydrolysate.

In some embodiments, at least one, two, three, four, five, or more (e.g., all) of (a)-(f) is selected from Table 1.

In some embodiments, the wt. ratio of the leucine amino acid entity, the arginine amino acid entity, the glutamine amino acid entity, and the NAC-amino acid entity is 1+/−20%:1.5+/−20%:2+/−20%:0.15+/−20%. In some embodiments, the wt. ratio of the leucine amino acid entity, the isoleucine amino acid entity, the valine amino acid entity, the arginine amino acid entity, the glutamine amino acid entity, and the NAC-amino acid entity is 1+/−20%:0.5+/−20%:0.5+/−20%:1.5+/−20%:2+/−20%:0.15+/−20%.

In some embodiments, the composition (e.g., the Active Moiety) comprises:

a) an leucine amino acid entity chosen from: i) L-leucine or a salt thereof, ii) a dipeptide or salt thereof, or tripeptide or salt thereof, comprising L-leucine, or iii) β-hydroxy-β-methylbutyrate (HMB) or a salt thereof;

b) an arginine amino acid entity chosen from: i) L-arginine or a salt thereof, ii) a dipeptide or salt thereof, or tripeptide or salt thereof, comprising L-arginine, iii) creatine or a salt thereof, or iv) a dipeptide or salt thereof, or tripeptide or salt thereof, comprising creatine;

c) the glutamine amino acid entity is L-glutamine or a salt thereof or a dipeptide or salt thereof, or tripeptide or salt thereof, comprising L-glutamine; and

d) the NAC entity is NAC or a salt thereof or a dipeptide or salt thereof, comprising NAC.

In some embodiments, the composition (e.g., the Active Moiety) further comprises one or both of: e) L-isoleucine or a salt thereof or a dipeptide or salt thereof, or tripeptide or salt thereof, comprising L-isoleucine; or f) L-valine or a salt thereof or a dipeptide or salt thereof, or tripeptide or salt thereof, comprising L-valine.

In some embodiments, the composition (e.g., the Active Moiety) comprises: a) the leucine amino acid entity is L-leucine or a salt thereof; b) the arginine amino acid entity is L-arginine or a salt thereof; c) the glutamine amino acid entity is L-glutamine or a salt thereof; and d) the NAC entity is NAC or a salt thereof.

In some embodiments, the composition (e.g., the Active Moiety) comprises: a) the leucine amino acid entity is L-leucine or a salt thereof; b) the arginine amino acid entity is L-arginine or a salt thereof; c) the glutamine amino acid entity is L-glutamine or a salt thereof; d) the NAC entity is NAC or a salt thereof; e) the isoleucine amino acid entity is L-isoleucine or a salt thereof; and f) the valine amino acid entity is L-valine or a salt thereof.

In some embodiments, the composition (e.g., the Active Moiety) is formulated with a pharmaceutically acceptable carrier.

In some embodiments, the composition (e.g., the Active Moiety) is formulated as a dietary composition.

### BRIEF DESCRIPTION OF THE DRAWINGS

FIGS. 1A-1B are graphs showing the effect of treatment with an amino acid composition (Amino Acid Composition A-1) on the NAFLD activity score, ballooning, and fibrosis in the STAM mouse model (FIG. 1A) and in the FATZO mouse model (FIG. 1B).

FIG. 2 is a schematic showing treatment regimens for administration of an amino acid composition to STAM and FATZO mice.

FIGS. 3A-3E are a series of graphs and images showing the effect of treating STAM and FATZO mice with an amino acid composition on the NAFLD activity score (NAS), steatosis, inflammation, and liver fibrosis as determined with histology.

FIG. 4 is an image of a gene map of the liver gene expression pattern following treatment with the amino acid composition in STAM mice showing suppression of the fibrogenic TGF-b signaling pathway.

FIG. 5 is a series of graphs showing MCP-1 and MIP-1 protein levels, which are the ligands of C-C chemokine receptor types 2 (CCR2) and 5 (CCR5), following treatment with the amino acid composition.

FIG. 6 is a series of microscopy images showing liver histology (H&E stain or Sirius Red stain for collagen deposition) from FATZO mice after administration of the indicated amino acid compositions.

FIG. 7 is a series of microscopy images showing liver histology from FATZO mice after administration of the indicated amino acid compositions.

FIG. 8 is a series of graphs showing NAFLD activity scores (top left panel), Sirius Red staining (top right panel), steatosis levels (bottom left panel), inflammation (bottom middle panel), and ballooning (bottom right panel) observed in fixed liver tissues from FATZO mice after administration of the indicated amino acid compositions.

FIGS. 9A-9B are a series of graphs showing the effect of treating human subjects with an amino acid composition on levels of proC3 (FIG. 9A) in addition to PIIINP and TIMP-1 (FIG. 9B).

### DETAILED DESCRIPTION

Described herein, in part, is a composition (e.g., an Active Moiety) comprising amino acid entities and methods of reducing fibrosis by administering an effective amount of the composition. The composition may be administered to treat or prevent a fibrotic condition or disorder in a subject in need thereof. The amino acid entities and relative amounts of the amino acid entities in the composition have been carefully selected, e.g., to reduce fibrosis in a subject (e.g., a subject having a fibrotic condition or disorder) that requires the coordination of many biological, cellular, and molecular processes. The composition allows for multi-pathway beneficial effects on tissue physiology to optimize modulation of signaling pathways involved in the fibrotic response and reduce deposition (and improve resorption) of extracellular matrix in fibrosis. In particular, the compositions have been specifically tailored to reduce fibrogenic gene/protein expression, reduce inflammation associated with fibrosis, and inhibit pathways associated with fibrosis.

In an example described in detail below, a composition of the invention improved fibrosis and reduced fibrogenic gene and protein expression.

### Definitions

Terms used in the claims and specification are defined as set forth below unless otherwise specified.

It must be noted that, as used in the specification and the appended claims, the singular forms “a,” “an” and “the” include plural referents unless the context clearly dictates otherwise.

As used herein, the term “amino acid entity” refers to a levo (L)-amino acid in free form or salt form (or both), the L-amino acid residue in a peptide smaller than 20 amino acid residues (e.g., oligopeptide, e.g., a dipeptide or a tripeptide), a derivative of the amino acid, a precursor of the amino acid, or a metabolite of the amino acid (see, e.g., Table 1). An amino acid entity includes a derivative of the amino acid, a precursor of the amino acid, a metabolite of the amino acid, or a salt form of the amino acid that is capable of effecting biological functionality of the free L-amino acid. An amino acid entity does not include a naturally occurring polypeptide or protein of greater than 20 amino acid residues, either in whole or modified form, e.g., hydrolyzed form.

Salts of amino acids include any ingestible salt. For pharmaceutical compositions, the salt form of an amino acid present in the composition (e.g., Active Moiety) should be a pharmaceutically acceptable salt. In a specific example, the salt form is the hydrochloride (HCl) salt form of the amino acid.

In some embodiments, the derivative of an amino acid entity comprises an amino acid ester (e.g., an alkyl ester, e.g., an ethyl ester or a methyl ester of an amino acid entity) or a keto-acid.

TABLE 1
Amino acid entities include amino acids, precursors, metabolites, and
derivatives of the compositions described herein.

| | Exemp- | | | |
|---|---|---|---|---|
| | lary | | | |
| | Amino | | | |
| | Acid | Precursors | Metabolites | Derivatives |
| Leucine | L-Leucine | Oxo-leucine | HMB (beta- | N-Acetyl- |
| | | | hydroxy-beta- | Leucine |
| | | | methybutyrate); | |
| | | | Oxo-leucine; | |
| | | | Isovaleryl-CoA | |
| Isoleucine | L- | 2-Oxo-3- | 2-Oxo-3- | N-Acetyl- |
| | Isoleucine | methyl- | methyl- | Isoleucine |
| | | valerate | valerate; | |
| | | | Methylbutyrl- | |
| | | | CoA | |
| Valine | L-Valine | 2-Oxo- | Isobutyrl-CoA | N-Acetyl- |
| | | valerate | | Valine |
| Arginine | L- | Arginino- | Agmatine; | N-Acetyl- |
| | Arginine | succinate; | Creatine | Arginine |
| | | Aspartate; | | |
| | | Glutamate | | |
| Glutamine | L- | Glutamate | Carbamoyl-P; | N-Acetyl- |
| | Glutamine | | Glutamate | Glutamine |
| NAC | N-Acetyl | Acetylserine; | Glutathione; | Cystine; |
| | cysteine | Cystathionine; | Cystathionine | Cysteamine |
| | | | Homocysteine; | |
| | | | Methionine | |
| Serine | L-Serine | Phospho- | Glycine, | |
| | | serine, P- | Tryptophan, | |
| | | hydroxy-, | Acetylserine, | |
| | | pyruvate | Cystathionine, | |
| | | L-Glycine | Phosphatidyl- | |
| | | | serine | |

“About” and “approximately” shall generally mean an acceptable degree of error for the quantity measured given the nature or precision of the measurements. Exemplary degrees of error are within 15 percent (%), typically, within 10%, and more typically, within 5% of a given value or range of values.

An “amino acid” refers to an organic compound having an amino group (—NH₂), a carboxylic acid group (—C(═O)OH), and a side chain bonded through a central carbon atom, and includes essential and non-essential amino acids and natural, non-proteinogenic, and unnatural amino acids.

As used herein, the term “Active Moiety” means a combination of four or more amino acid entities that, in aggregate, have the ability to have a physiological effect as described herein, e.g., an anti-fibrotic effect. For example, an Active Moiety can rebalance a metabolic dysfunction in a subject suffering from a disease or disorder. An Active Moiety of the invention can contain other biologically active ingredients. In some examples, the Active Moiety comprises a defined combination of four or more amino acid entities, as set out in detail below. In other embodiments, the Active Moiety consists of a defined combination of amino acid entities, as set out in detail below.

The individual amino acid entities are present in the composition, e.g., Active Moiety, in various amounts or ratios, which can be presented as amount by weight (e.g., in grams), ratio by weight of amino acid entities to each other, amount by mole, amount by weight percent of the composition, amount by mole percent of the composition, caloric content, percent caloric contribution to the composition, etc. Generally this disclosure will provide grams of amino acid entity in a dosage form, weight percent of an amino acid entity relative to the weight of the composition, i.e., the weight of all the amino acid entities and any other biologically active ingredient present in the composition, or in ratios. In some embodiments, the composition, e.g., Active Moiety, is provided as a pharmaceutically acceptable preparation (e.g., a pharmaceutical product).

The term “effective amount” as used herein means an amount of an active of the invention in a composition of the invention, particularly a pharmaceutical composition of the invention, which is sufficient to reduce a symptom and/or improve a condition to be treated (e.g., provide a desired clinical response). The effective amount of an active for use in a composition will vary with the particular condition being treated, the severity of the condition, the duration of treatment, the nature of concurrent therapy, the particular active being employed, the particular pharmaceutically-acceptable excipient(s) and/or carrier(s) utilized, and like factors with the knowledge and expertise of the attending physician.

A “pharmaceutical composition” described herein comprises at least one “Active Moiety” and a pharmaceutically acceptable carrier or excipient. In some embodiments, the pharmaceutical composition is used as a therapeutic. Other compositions, which need not meet pharmaceutical standards (GMP; pharmaceutical grade components) can be used as a nutraceutical, a medical food, or as a supplement, these are termed “consumer health compositions”.

The term “pharmaceutically acceptable” as used herein, refers to amino acids, materials, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In a specific embodiment, “pharmaceutically acceptable” means free of detectable endotoxin or endotoxin levels are below levels acceptable in pharmaceutical products.

In a specific embodiment, “pharmaceutically acceptable” means a standard used by the pharmaceutical industry or by agencies or entities (e.g., government or trade agencies or entities) regulating the pharmaceutical industry to ensure one or more product quality parameters are within acceptable ranges for a medicine, pharmaceutical composition, treatment, or other therapeutic. A product quality parameter can be any parameter regulated by the pharmaceutical industry or by agencies or entities, e.g., government or trade agencies or entities, including but not limited to composition; composition uniformity; dosage; dosage uniformity; presence, absence, and/or level of contaminants or impurities; and level of sterility (e.g., the presence, absence and/or level of microbes). Exemplary government regulatory agencies include: Federal Drug Administration (FDA), European Medicines Agency (EMA), SwissMedic, China Food and Drug Administration (CFDA), or Japanese Pharmaceuticals and Medical Devices Agency (PMDA).

The term “pharmaceutically acceptable excipient” refers to an ingredient in a pharmaceutical formulation, other than an active, which is physiologically compatible. A pharmaceutically acceptable excipient can include, but is not limited to, a buffer, a sweetener, a dispersion enhancer, a flavoring agent, a bitterness masking agent, a natural coloring, an artificial coloring, a stabilizer, a solvent, or a preservative. In a specific embodiment, a pharmaceutically acceptable excipient includes one or both of citric acid or lecithin.

The term “non-amino acid entity protein component,” as used herein, refers to a peptide (e.g., a polypeptide or an oligopeptide), a fragment thereof, or a degraded peptide. Exemplary non-amino acid entity protein components include, but are not limited to, one or more of whey protein, egg white protein, soy protein, casein, hemp protein, pea protein, brown rice protein, or a fragment or degraded peptide thereof.

The term “non-protein component,” as used herein, refers to any component of a composition other than a protein component. Exemplary non-protein components can include, but are not limited to, a saccharide (e.g., a monosaccharide (e.g., dextrose, glucose, or fructose), a disaccharide, an oligosaccharide, or a polysaccharide); a lipid (e.g., a sulfur-containing lipid (e.g., alpha-lipoic acid), a long chain triglyceride, an omega 3 fatty acid (e.g., EPA, DHA, STA, DPA, or ALA), an omega 6 fatty acid (GLA, DGLA, or LA), a medium chain triglyceride, or a medium chain fatty acid); a vitamin (e.g., vitamin A, vitamin E, vitamin C, vitamin D, vitamin B6, vitamin B12, biotin, or pantothenic acid); a mineral (zinc, selenium, iron, copper, folate, phosphorous, potassium, manganese, chromium, calcium, or magnesium); or a sterol (e.g., cholesterol).

A composition, formulation or product is “therapeutic” if it provides a desired clinical effect. A desired clinical effect can be shown by lessening the progression of a disease and/or alleviating one or more symptoms of the disease.

A “unit dose” or “unit dosage” comprises the drug product or drug products in the form in which they are marketed for use, with a specific mixture of the active and inactive components (excipients), in a particular configuration (e.g, a capsule shell, for example), and apportioned into a particular dose (e.g., in multiple stick packs).

As used herein, the terms “treat,” “treating,” or “treatment” of fibrosis (e.g. a fibrotic condition or disorder) refers to ameliorating fibrosis (e.g., slowing, arresting, or reducing the development of fibrosis or at least one of the clinical symptoms thereof); alleviating or ameliorating at least one physical parameter including those which may not be discernible by the patient; and/or preventing or delaying the onset or development or progression of fibrosis.

### Compositions Comprising Amino Acid Entities (e.g., Active Moieties)

The composition of the invention as described herein (e.g., an Active Moiety) comprises amino acid entities, e.g., the amino acid entities shown in Table 1.

In certain embodiments, the leucine amino acid entity is chosen from L-leucine, β-hydroxy-β-methylbutyrate (HMB), oxo-leucine (α-ketoisocaproate (KIC)), isovaleryl-CoA, n-acetyl-leucine, or a combination thereof.

In certain embodiments, the arginine amino acid entity is chosen from L-arginine, creatine, argininosuccinate, aspartate, glutamate, agmatine, N-acetyl-arginine, or a combination thereof.

In certain embodiments, the glutamine amino acid entity is chosen from L-glutamine, glutamate, carbamoyl-P, glutamate, n-acetylglutamine, or a combination thereof.

In certain embodiments, the NAC-amino acid entity is selected chosen from NAC, acetylserine, cystathionine, cystathionine, homocysteine, glutathione, or a combination thereof.

In certain embodiments, the isoleucine amino acid entity is chosen from L-isoleucine, 2-oxo-3-methyl-valerate (α-keto-beta-methylvaleric acid (KMV)), methylbutyrl-CoA, N-acetyl-isoleucine, or a combination thereof.

In certain embodiments, the valine amino acid entity chosen from L-valine, 2-oxo-valerate (α-ketoisovalerate (KIV)), isobutyrl-CoA, N-acetyl-valine, or a combination thereof.

In certain embodiments, the serine amino acid entity is chosen from L-serine, phosphoserine, p-hydroxypyruvate, glycine, acetylserine, cystathionine, phosphatidylserine, or a combination thereof. In some embodiments, the serine amino acid entity is chosen from L-serine or L-glycine. In one embodiment, the serine amino acid entity is L-serine. In another embodiment, the serine amino acid entity is L-glycine. In another embodiment, the serine amino acid entity is L-glycine and L-serine (e.g., L-glycine and L-serine at a wt. ratio of 1:1).

The composition described herein can further comprise one, two, three, four, five, or more (e.g., all) or more of L-serine, L-glycine, creatine, or glutathione.

In some embodiments, the composition comprises an leucine amino acid entity, an isoleucine amino acid entity, an valine amino acid entity, an arginine amino acid entity, a glutamine amino acid entity (e.g., L-glutamine or a salt thereof), a NAC-entity, and L-serine.

In some embodiments, the composition comprises an leucine amino acid entity, an isoleucine amino acid entity, an valine amino acid entity, an arginine amino acid entity, a glutamine amino acid entity (e.g., L-glutamine or a salt thereof), a NAC-entity, and L-glycine.

In some embodiments, the composition comprises an leucine amino acid entity, an isoleucine amino acid entity, an valine amino acid entity, an arginine amino acid entity, a glutamine amino acid entity (e.g., L-glutamine or a salt thereof), a NAC-entity, L-glycine, and L-serine.

In some embodiments, the composition comprises an leucine amino acid entity, an isoleucine amino acid entity, an valine amino acid entity, an arginine amino acid entity, a glutamine amino acid entity (e.g., L-glutamine or a salt thereof), and a NAC-entity. In some embodiments, one, two, three, four, five, or more (e.g., all) of (a)-(f) are in free amino acid form in the composition, e.g., at least: 42 wt. %, 75 wt. %, 90 wt. %, or more of the total wt. of amino acid entity components or total components is one, two, three, four, five, or more (e.g., all) of (a)-(f) in free amino acid form in the composition (e.g., in dry form).

In some embodiments, one, two, three, four, five, or more (e.g., all) of (a)-(f) is in salt form in the composition, e.g., at least: 0.01 wt. %, 0.1 wt. %, 0.5 wt. %, 1 wt. %, 5 wt. %, or 10 wt. %, or more of the total wt. of amino acid entity components or total components is one, two, three, four, five, or more (e.g., all) of (a)-(f) in salt form in the composition.

In some embodiments, one, two, three, four, five, or more (e.g., all) of (a)-(f) is provided as part of a dipeptide or tripeptide, e.g., in an amount of at least: 0.01 wt. %, 0.1 wt. %, 0.5 wt. %, 1 wt. %, 5 wt. %, or 10 wt. %, or more of amino acid entity components or total components of the composition.

In some embodiments, the composition comprises, consists essentially of, or consists of:

a) a leucine amino acid entity,

b) a arginine amino acid entity,

c) glutamine amino acid entity; and

d) a N-acetylcysteine (NAC) entity.

In some embodiments, the composition (e.g., the Active Moiety) comprises, consists essentially of, or consists of:

a) an leucine amino acid entity chosen from: i) L-leucine or a salt thereof, ii) a dipeptide or salt thereof, or tripeptide or salt thereof, comprising L-leucine, or iii) β-hydroxy-β-methylbutyrate (HMB) or a salt thereof;

b) an arginine amino acid entity chosen from: i) L-arginine or a salt thereof, ii) a dipeptide or salt thereof, or tripeptide or salt thereof, comprising L-arginine, iii) creatine or a salt thereof, or v) a dipeptide or salt thereof, or tripeptide or salt thereof, comprising creatine;

c) the glutamine amino acid entity is L-glutamine or a salt thereof or a dipeptide or salt thereof, or tripeptide or salt thereof, comprising L-glutamine; and

d) the NAC entity is NAC or a salt thereof or a dipeptide or salt thereof, comprising NAC.

In some embodiments, the composition (e.g., the Active Moiety) further comprises, consists essentially of, or consists of one or both of: e) L-isoleucine or a salt thereof or a dipeptide or salt thereof, or tripeptide or salt thereof, comprising L-isoleucine; or f) L-valine or a salt thereof or a dipeptide or salt thereof, or tripeptide or salt thereof, comprising L-valine.

In some embodiments, the composition (e.g., the Active Moiety) comprises, consists essentially of, or consists of: a) L-leucine or a salt thereof; b) L-arginine or a salt thereof; c) L-glutamine or a salt thereof; and d) NAC or a salt thereof.

In some embodiments, the composition (e.g., the Active Moiety) is capable of reducing or preventing fibrosis. For instance, the one or both of reducing or inhibiting fibrosis comprises reducing a level of one or both of collagen, e.g., type I and III collagen or α-smooth muscle actin (aSMA).

In certain embodiments, the composition (e.g., the Active Moiety) is capable of reducing, or reduces, fibrosis by at least 5%, 10%, or 15%, as detected using an assay of hydroxyproline, e.g., an antibody-based detection assay, e.g., an ELISA, e.g., as described in Example 1, e.g., relative to a reference composition (e.g., a vehicle control).

In certain embodiments, the composition (e.g., the Active Moiety) is capable of reducing, or reduces, liver fibrosis or liver injury by at least 20%, 50%, or 65%, as detected using LX-2 cells, e.g., levels of Col1a1, and/or TIMP2 in LX-2 cells, e.g., as assessed using a nucleic acid amplification method, e.g., PCR or qRT-PCR, e.g., as described in Example 3, e.g., relative to a reference composition (e.g., a vehicle control, single amino acid entity, or combination of amino acid entities).

In some embodiments, the composition (e.g., the Active Moiety) is capable of reducing, or reduces, fibrosis in one or more liver cell types (e.g., one, two, or three of hepatocyte cells, stellate cells, or macrophages, e.g., in a triculture of hepatocyte cells, stellate cells, and macrophages), e.g., as detected by a change (e.g., a decrease) in a level of a fibrotic marker, e.g., one, two, three, or more (e.g., all) of procollagen Iα1, MCP-1, YKL40, or GROalpha (CXCL1)), e.g., by at least 20%, 30%, 40%, or 50%, e.g., as assessed using an antibody-based detection assay, e.g., an ELISA, e.g., as described in Example 9, e.g., relative to a reference composition (e.g., a lower concentration of the composition, a vehicle control, a single amino acid entity, or a combination of amino acid entities). In certain embodiments, the composition results in a decrease of one, two, three, or more (e.g., all) of:
(i) a level of procollagen Iα1 (e.g., a decrease in the level of procollagen Iα1 of at least 20%, 30%, 40%, or 50%);
(ii) a level of MCP-1 (e.g., a decrease in the level of MCP-1 of at least 50%, 60%, 70%, 80%, or 90%);
(iii) a level of YKL40 (e.g., a decrease in the level of YKL40 of at least 70%, 80%, 90%, or 95%); or
(iv) a level of GROalpha (CXCL1) (e.g., a decrease in the level of GROalpha (CXCL1) of at least 15%, 20%, 25%, or 30%).

In some embodiments, the activity of the composition (e.g., the Active Moiety) is assessed by contacting one or more liver cell types (e.g., one, two, or three of hepatocyte cells, stellate cells, or macrophages), e.g. liver cell types separated by a membrane (e.g., a permeable membrane, e.g., a Transwell) in culture (e.g., hepatocyte cells separated by a membrane from one or both of stellate cells or macrophages) with the composition under the conditions described in Example 9.

In certain embodiments, the composition (e.g., the Active Moiety) is capable of reducing, or reduces, liver fibrosis or liver injury as detected by proliferation of stellate cells, e.g., levels of DNA synthesis in stellate cells, e.g., by at least 50%, 60%, 70%, or 80%, e.g., as assessed using a nuclei stain, e.g., EdU (5-ethynyl-2′-deoxyuridine), e.g., as described in Example 10, e.g., relative to a reference composition (e.g., a vehicle control (PBS), a single amino acid entity, or combination of amino acid entities).

i. Amounts

The composition (e.g., the Active Moiety) can include 0.5 g+/−20% to 10 g+/−20% of an leucine amino acid entity, 1 g+/−20% to 15 g+/−20% of an arginine amino acid entity, 0.5 g+/−20% to 20 g+/−20% of a glutamine amino acid entity, and 0.1 g+/−20% to 5 g+/−20% of a NAC-entity.

An exemplary composition can include 1 g of an leucine amino acid entity, 0.5 g of an isoleucine amino acid entity, 0.5 g of a valine amino acid entity, 1.5 g or 1.81 g of an arginine amino acid entity, 2 g of a glutamine amino acid entity, and 0.15 g of a NAC-entity (e.g., g/packet as shown in Table 2).

TABLE 2
Exemplary composition including the form L-arginine (R) or L-arginine HCl (R HCl)).

| | wt. ratio | wt. ratio | wt. % | wt. % | g /packet | g /packet | g dose #1 | g dose #1 | g dose #2 | g dose #2 |
|---|---|---|---|---|---|---|---|---|---|---|
| Amino acid | (R) | (R HCl) | (R) | (R HCl) | (R) | (R HCl) | (R) | (R HCl) | (R) | (R HCl) |
| Leucine | 1 | 1 | 17.70 | 16.78 | 1.00 g | 1.00 g | 2 | 2 g | 4 | 4 g |
| Isoleucine | 0.5 | 0.5 | 8.85 | 8.39 | 0.50 g | 0.50 g | 1 | 1 g | 2 | 2 g |
| Valine | 0.5 | 0.5 | 8.85 | 8.39 | 0.50 g | 0.50 g | 1 | 1 g | 2 | 2 g |
| Arginine | 1.5 | 1.81 | 26.55 | 30.37 | 1.5 g | 1.81 g | 3 | 3.62 g | 6 | 7.24 g |
| Glutamine | 2 | 2 | 35.4 | 33.56 | 2.00 g | 2.00 g | 4 | 4 g | 8 | 8 g |
| NAC | 0.15 | 0.15 | 2.65 | 2.52 | 0.15 g | 0.15 g | 0.3 | 0.3 g | 0.6 | 0.6 g |
| Total AAs | | | | | 5.65 g | 5.96 g | 11.3 g | ~12 g | 2. | ~24 g |
| | | | | | | | | | 2.6 g | |

In some embodiments, the composition (e.g., the Active Moiety) includes 1 g+/−20% of an leucine amino acid entity, 0.5 g+/−20% of an isoleucine amino acid entity, 0.5+/−20% g of a valine amino acid entity, 1.5 g+/−20% of an arginine amino acid entity, 2 g+/−20% of a glutamine amino acid entity, and 0.15 g+/−20% of a NAC-entity. In some embodiments, the composition includes 1 g+/−15% of an leucine amino acid entity, 0.5 g+/−15% of an isoleucine amino acid entity, 0.5+/−15% g of a valine amino acid entity, 1.5 g+/−15% of an arginine amino acid entity, 2 g+/−15% of a glutamine amino acid entity, and 0.15 g+/−15% of a NAC-entity. In some embodiments, the composition includes 1 g+/−10% of an leucine amino acid entity, 0.5 g+/−10% of an isoleucine amino acid entity, 0.5+/−10% g of a valine amino acid entity, 1.5 g+/−10% of an arginine amino acid entity, 2 g+/−10% of a glutamine amino acid entity, and 0.15 g+/−10% of a NAC-entity. In some embodiments, the composition includes 1 g+/−5% of an leucine amino acid entity, 0.5 g+/−5% of an isoleucine amino acid entity, 0.5+/−5% g of a valine amino acid entity, 1.5 g+/−5% of an arginine amino acid entity, 2 g+/−5% of a glutamine amino acid entity, and 0.15 g+/−5% of a NAC-entity. In some embodiments, the composition includes 1 g of an leucine amino acid entity, 0.5 g of an isoleucine amino acid entity, 0.5 g of a valine amino acid entity, 1.5 g or 1.81 g of an arginine amino acid entity, 2 g of a glutamine amino acid entity, and 0.15 g of a NAC-entity.

In some embodiments, the composition (e.g., the Active Moiety) includes 1 g+/−20% of an leucine amino acid entity, 0.5 g+/−20% of an isoleucine amino acid entity, 0.5+/−20% g of a valine amino acid entity, 1.5 g+/−20% of an arginine amino acid entity, 2 g+/−20% of a glutamine amino acid entity, and 0.3 g+/−20% of a NAC-entity. In some embodiments, the composition includes 1 g+/−15% of an leucine amino acid entity, 0.5 g+/−15% of an isoleucine amino acid entity, 0.5+/−15% g of a valine amino acid entity, 1.5 g+/−15% of an arginine amino acid entity, 2 g+/−15% of a glutamine amino acid entity, and 0.3 g+/−15% of a NAC-entity. In some embodiments, the composition includes 1 g+/−10% of an leucine amino acid entity, 0.5 g+/−10% of an isoleucine amino acid entity, 0.5+/−10% g of a valine amino acid entity, 1.5 g+/−10% of an arginine amino acid entity, 2 g+/−10% of a glutamine amino acid entity, and 0.3 g+/−10% of a NAC-entity. In some embodiments, the composition includes 1 g+/−5% of an leucine amino acid entity, 0.5 g+/−5% of an isoleucine amino acid entity, 0.5+/−5% g of a valine amino acid entity, 1.5 g+/−5% of an arginine amino acid entity, 2 g+/−5% of a glutamine amino acid entity, and 0.3 g+/−5% of a NAC-entity. In some embodiments, the composition includes 1 g of an leucine amino acid entity, 0.5 g of an isoleucine amino acid entity, 0.5 g of a valine amino acid entity, 1.5 g or 1.81 g of an arginine amino acid entity, 2 g of a glutamine amino acid entity, and 0.3 g of a NAC-entity.

An exemplary composition can include 1 g of an leucine amino acid entity, 0.5 g of an isoleucine amino acid entity, 0.5 g of a valine amino acid entity, 0.75 g or 0.905 g of an arginine amino acid entity, 2 g of a glutamine amino acid entity, and 0.15 g of a NAC-entity (e.g., g/packet as shown in Table 3).

TABLE 3
Exemplary composition including the form L-arginine (R) or L-arginine HCl (R HCl)).

| Amino | wt. ratio | wt. ratio | wt. % | wt. % | g /packet | g /packet | g dose #1 | g dose #1 | g dose #2 | g dose #2 |
|---|---|---|---|---|---|---|---|---|---|---|
| acid | (R) | (R HCl) | (R) | (R HCl) | (R) | (R HCl) | (R) | (R HCl) | (R) | (R HCl) |
| Leucine | 1 | 1 | 20.41 | 19.78 | 1.00 g | 1.00 g | 2 | 2 g | 4 | 4 g |
| Isoleucine | 0.5 | 0.5 | 10.20 | 9.89 | 0.50 g | 0.50 g | 1 | 1 g | 2 | 2 g |
| Valine | 0.5 | 0.5 | 10.20 | 9.89 | 0.50 g | 0.50 g | 1 | 1 g | 2 | 2 g |
| Arginine | 0.75 | 0.905 | 15.31 | 17.90 | 0.75 g | 0.905 g | 1.5 | 1.81 g | 3 | 3.62 g |
| Glutamine | 2 | 2 | 40.82 | 39.56 | 2.00 g | 2.00 g | 4 | 4 g | 8 | 8 g |
| NAC | 0.15 | 0.15 | 3.06 | 2.97 | 0.15 g | 0.15 g | 0.3 | 0.3 g | 0.6 | 0.6 g |
| Total AAs | | | | | 4.9 g | 5.06 g | 9.8 g | ~10 g | 19.6 g | ~20 g |

In some embodiments, the composition (e.g., the Active Moiety) includes 1 g+/−20% of an leucine amino acid entity, 0.5 g+/−20% of an isoleucine amino acid entity, 0.5+/−20% g of a valine amino acid entity, 0.75 g+/−20% of an arginine amino acid entity, 2 g+/−20% of a glutamine amino acid entity, and 0.15 g+/−20% of a NAC-entity. In some embodiments, the composition includes 1 g+/−15% of an leucine amino acid entity, 0.5 g+/−15% of an isoleucine amino acid entity, 0.5+/−15% g of a valine amino acid entity, 0.75 g+/−15% of an arginine amino acid entity, 2 g+/−15% of a glutamine amino acid entity, and 0.15 g+/−15% of a NAC-entity. In some embodiments, the composition includes 1 g+/−10% of an leucine amino acid entity, 0.5 g+/−10% of an isoleucine amino acid entity, 0.5+/−10% g of a valine amino acid entity, 0.75 g+/−10% of an arginine amino acid entity, 2 g+/−10% of a glutamine amino acid entity, and 0.15 g+/−10% of a NAC-entity. In some embodiments, the composition includes 1 g+/−5% of an leucine amino acid entity, 0.5 g+/−5% of an isoleucine amino acid entity, 0.5+/−5% g of a valine amino acid entity, 0.75 g+/−5% of an arginine amino acid entity, 2 g+/−5% of a glutamine amino acid entity, and 0.15 g+/−5% of a NAC-entity. In some embodiments, the composition includes 1 g of an leucine amino acid entity, 0.5 g of an isoleucine amino acid entity, 0.5 g of a valine amino acid entity, 0.75 g or 0.905 g of an arginine amino acid entity, 2 g of a glutamine amino acid entity, and 0.15 g of a NAC-entity.

In some embodiments, the composition (e.g., the Active Moiety) includes 1 g+/−20% of an leucine amino acid entity, 0.5 g+/−20% of an isoleucine amino acid entity, 0.5+/−20% g of a valine amino acid entity, 0.75 g+/−20% of an arginine amino acid entity, 2 g+/−20% of a glutamine amino acid entity, and 0.3 g+/−20% of a NAC-entity. In some embodiments, the composition includes 1 g+/−15% of an leucine amino acid entity, 0.5 g+/−15% of an isoleucine amino acid entity, 0.5+/−15% g of a valine amino acid entity, 0.75 g+/−15% of an arginine amino acid entity, 2 g+/−15% of a glutamine amino acid entity, and 0.3 g+/−15% of a NAC-entity. In some embodiments, the composition includes 1 g+/−10% of an leucine amino acid entity, 0.5 g+/−10% of an isoleucine amino acid entity, 0.5+/−10% g of a valine amino acid entity, 0.75 g+/−10% of an arginine amino acid entity, 2 g+/−10% of a glutamine amino acid entity, and 0.3 g+/−10% of a NAC-entity. In some embodiments, the composition includes 1 g+/−5% of an leucine amino acid entity, 0.5 g+/−5% of an isoleucine amino acid entity, 0.5+/−5% g of a valine amino acid entity, 0.75 g+/−5% of an arginine amino acid entity, 2 g+/−5% of a glutamine amino acid entity, and 0.3 g+/−5% of a NAC-entity. In some embodiments, the composition includes 1 g of an leucine amino acid entity, 0.5 g of an isoleucine amino acid entity, 0.5 g of a valine amino acid entity, 0.75 g or 0.905 g of an arginine amino acid entity, 2 g of a glutamine amino acid entity, and 0.3 g of a NAC-entity.

An exemplary composition can include 1 g of an leucine amino acid entity, 0.5 g of an isoleucine amino acid entity, 0.25 g of a valine amino acid entity, 0.75 g or 0.905 g of an arginine amino acid entity, 1 g of a glutamine amino acid entity, and 0.225 g of a NAC-entity (e.g., g/packet as shown in Table 4).

TABLE 4
Exemplary composition including the form L-arginine (R) or L-arginine HCl (R HCl)).

| Amino | wt. ratio | wt. ratio | wt. % | wt. % | g /packet | g /packet | g dose #1 | g dose #1 | g dose #2 | g dose #2 |
|---|---|---|---|---|---|---|---|---|---|---|
| acid | (R) | (R HCl) | (R) | (R HCl) | (R) | (R HCl) | (R) | (R HCl) | (R) | (R HCl) |
| Leucine | 1 | 1 | 26.85 | 25.77 | 1.00 g | 1.00 g | 2 | 2 g | 4 | 4 g |
| Isoleucine | 0.5 | 0.5 | 13.42 | 12.89 | 0.50 g | 0.50 g | 1 | 1 g | 2 | 2 g |
| Valine | 0.25 | 0.25 | 6.71 | 6.44 | 0.25 g | 0.25 g | 0.5 | 0.50 g | 1 | 1 g |
| Arginine | 0.75 | 0.905 | 20.13 | 23.32 | 0.75 g | 0.905 g | 1.5 | 1.81 g | 3 | 3.62 g |
| Glutamine | 1 | 1 | 26.85 | 25.77 | 1.00 g | 1.00 g | 2 | 2 g | 4 | 4 g |
| NAC | 0.225 | 0.225 | 6.04 | 5.80 | 0.225 g | 0.225 g | 0.45 | 0.45 g | 0.9 | 0.9 g |
| Total AAs | | | | | 3.725 g | 3.88 g | 7.45 g | 7.76 g | 14.9 g | 15.52 g |

In some embodiments, the composition (e.g., the Active Moiety) includes 1 g+/−20% of an leucine amino acid entity, 0.5 g+/−20% of an isoleucine amino acid entity, 0.25+/−20% g of a valine amino acid entity, 0.75 g+/−20% of an arginine amino acid entity, 1 g+/−20% of a glutamine amino acid entity, and 0.225 g+/−20% of a NAC-entity. In some embodiments, the composition includes 1 g+/−15% of an leucine amino acid entity, 0.5 g+/−20% of an isoleucine amino acid entity, 0.25+/−20% g of a valine amino acid entity, 0.75 g+/−15% of an arginine amino acid entity, 1 g+/−15% of a glutamine amino acid entity, and 0.225 g+/−15% of a NAC-entity. In some embodiments, the composition includes 1 g+/−10% of an leucine amino acid entity, 0.5 g+/−20% of an isoleucine amino acid entity, 0.25+/−20% g of a valine amino acid entity, 0.75 g+/−10% of an arginine amino acid entity, 1 g+/−10% of a glutamine amino acid entity, and 0.225 g+/−10% of a NAC-entity. In some embodiments, the composition includes 1 g+/−5% of an leucine amino acid entity, 0.5 g+/−20% of an isoleucine amino acid entity, 0.25+/−20% g of a valine amino acid entity, 0.75 g+/−5% of an arginine amino acid entity, 1 g+/−5% of a glutamine amino acid entity, and 0.225 g+/−5% of a NAC-entity. An exemplary composition can include 1 g of an leucine amino acid entity, 0.5 g of an isoleucine amino acid entity, 0.25 g of a valine amino acid entity, 0.75 g or 0.905 g of an arginine amino acid entity, 1 g of a glutamine amino acid entity, 0.225 g of a NAC-entity, and 1.5 g of the serine amino acid entity (e.g., g/packet as shown in Table 5).

TABLE 5
Exemplary composition including the form L-arginine (R) or L-arginine HCl (R HCl)).

| Amino | wt. ratio | wt. ratio | wt. % | wt. % | g /packet | g /packet | g dose #1 | g dose #1 | g dose #2 | g dose #2 |
|---|---|---|---|---|---|---|---|---|---|---|
| acid | (R) | (R HCl) | (R) | (R HCl) | (R) | (R HCl) | (R) | (R HCl) | (R) | (R HCl) |
| Leucine | 1 | 1 | 19.14 | 18.59 | 1.00 g | 1.00 g | 2 | 2 g | 4 | 4 g |
| Isoleucine | 0.5 | 0.5 | 9.57 | 9.29 | 0.50 g | 0.50 g | 1 | 1 g | 2 | 2 g |
| Valine | 0.25 | 0.25 | 4.78 | 4.65 | 0.25 g | 0.25 g | 0.5 | 0.50 g | 1 | 1 g |
| Arginine | 0.75 | 0.905 | 14.35 | 16.82 | 0.75 g | 0.905 g | 1.5 | 1.81 g | 3 | 3.62 g |
| Glutamine | 1 | 1 | 19.14 | 18.59 | 1.00 g | 1.00 g | 2 | 2 g | 4 | 4 g |
| NAC | 0.225 | 0.225 | 4.31 | 4.18 | 0.225 g | 0.225 g | 0.45 | 0.45 g | 0.9 | 0.9 g |
| Serine | 1.5 | 1.5 | 28.71 | 27.88 | 1.5 | 1.5 | 3 | 3 | 6 | 6 |
| Total AAs | | | | | 5.225 | 5.38 g | 10.45 | 10.76 g | 20.9 | 21.52 g |

In some embodiments, the composition comprises 1 g+/−20% of the leucine amino acid entity, 0.5 g+/−20% of the isoleucine amino acid entity, 0.25 g+/−20% of the valine amino acid entity, 0.75 g+/−20% of the arginine amino acid entity, 1 g+/−20% of the glutamine amino acid entity, 0.225 g+/−20% of the NAC-amino acid entity, and 1.5 g+/−20% of the serine amino acid entity. In some embodiments, the composition comprises 1 g+/−15% of the leucine amino acid entity, 0.5 g+/−15% of the isoleucine amino acid entity, 0.25 g+/−15% of the valine amino acid entity, 0.75 g+/−15% of the arginine amino acid entity, 1 g+/−15% of the glutamine amino acid entity, 0.225 g+/−15% of the NAC-amino acid entity, and 1.5 g+/−15% of the serine amino acid entity. In some embodiments, the composition comprises 1 g+/−10% of the leucine amino acid entity, 0.5 g+/−10% of the isoleucine amino acid entity, 0.25 g+/−10% of the valine amino acid entity, 0.75 g+/−10% of the arginine amino acid entity, 1 g+/−10% of the glutamine amino acid entity, 0.225 g+/−10% of the NAC-amino acid entity, and 1.5 g+/−10% of the serine amino acid entity. In some embodiments, the composition comprises 1 g+/−5% of the leucine amino acid entity, 0.5 g+/−5% of the isoleucine amino acid entity, 0.25 g+/−5% of the valine amino acid entity, 0.75 g+/−5% of the arginine amino acid entity, 1 g+/−5% of the glutamine amino acid entity, 0.225 g+/−5% of the NAC-amino acid entity, and 1.5 g+/−5% of the serine amino acid entity.

An exemplary composition can include 1 g of an leucine amino acid entity, 0.5 g of an isoleucine amino acid entity, 0.25 g of a valine amino acid entity, 0.75 g or 0.905 g of an arginine amino acid entity, 1 g of a glutamine amino acid entity, 0.225 g of a NAC-entity, and 1.667 g of the serine amino acid entity (e.g., g/packet as shown in Table 6).

TABLE 6
Exemplary composition including the form L-arginine (R) or L-arginine HCl (R HCl)).

| | wt. ratio | wt. ratio | wt. % | wt. % | g/packet | | g/packet | | g dose #1 | | g dose #1 | | g dose #2 | | g dose #2 | |
|---|---|---|---|---|---|---|---|---|---|---|---|---|---|---|---|---|
| Amino acid | (R) | (R HCl) | (R) | (R HCl) | (R) | | (R HCl) | | (R) | | (R HCl) | | (R) | | (R HCl) | |
| Leucine | 1 | 1 | 18.54 | 18.02 | 1.00 | g | 1.00 | g | 2 | | 2 | g | 4 | | 4 | g |
| Isoleucine | 0.5 | 0.5 | 9.27 | 9.01 | 0.50 | g | 0.50 | g | 1 | | 1 | g | 2 | | 2 | g |
| Valine | 0.25 | 0.25 | 4.64 | 4.50 | 0.25 | g | 0.25 | g | 0.5 | | 0.50 | g | 1 | | 1 | g |
| Arginine | 0.75 | 0.905 | 13.91 | 16.31 | 0.75 | g | 0.905 | g | 1.5 | | 1.81 | g | 3 | | 3.62 | g |
| Glutamine | 1 | 1 | 18.54 | 18.02 | 1.00 | g | 1.00 | g | 2 | | 2 | g | 4 | | 4 | g |
| NAC | 0.225 | 0.225 | 4.17 | 4.05 | 0.225 | g | 0.225 | g | 0.45 | | 0.45 | g | 0.9 | | 0.9 | g |
| Serine | 1.667 | 1.667 | 30.92 | 30.09 | 1.67 | g | 1.67 | g | 3.33 | g | 3.33 | g | 6.67 | g | 6.67 | g |
| Total AAs | | | | | 5.395 | g | 5.55 | g | 10.78 | g | 11.09 | g | 21.57 | g | 22.19 | g |

In some embodiments, the composition comprises 1 g+/−20% of the leucine amino acid entity, 0.5 g+/−20% of the isoleucine amino acid entity, 0.25 g+/−20% of the valine amino acid entity, 0.75 g+/−20% of the arginine amino acid entity, 1 g+/−20% of the glutamine amino acid entity, 0.225 g+/−20% of the NAC-amino acid entity, and 1.667 g+/−20% of the serine amino acid entity. In some embodiments, the composition comprises 1 g+/−15% of the leucine amino acid entity, 0.5 g+/−15% of the isoleucine amino acid entity, 0.25 g+/−15% of the valine amino acid entity, 0.75 g+/−15% of the arginine amino acid entity, 1 g+/−15% of the glutamine amino acid entity, 0.225 g+/−15% of the NAC-amino acid entity, and 1.667 g+/−15% of the serine amino acid entity. In some embodiments, the composition comprises 1 g+/−10% of the leucine amino acid entity, 0.5 g+/−10% of the isoleucine amino acid entity, 0.25 g+/−10% of the valine amino acid entity, 0.75 g+/−10% of the arginine amino acid entity, 1 g+/−10% of the glutamine amino acid entity, 0.225 g+/−10% of the NAC-amino acid entity, and 1.667 g+/−10% of the serine amino acid entity. In some embodiments, the composition comprises 1 g+/−5% of the leucine amino acid entity, 0.5 g+/−5% of the isoleucine amino acid entity, 0.25 g+/−5% of the valine amino acid entity, 0.75 g+/−5% of the arginine amino acid entity, 1 g+/−5% of the glutamine amino acid entity, 0.225 g+/−5% of the NAC-amino acid entity, and 1.667 g+/−5% of the serine amino acid entity.

ii. Ratios

An exemplary composition can include a weight (wt.) ratio of the leucine amino acid entity, the isoleucine amino acid entity, the valine amino acid entity, the arginine amino acid entity, the glutamine amino acid entity, and the NAC-amino acid entity of 1+/−15%:0.5+/−15%:0.5+/−15%:1.5+/−15%:2+/−15%:0.15+/−15% or 1+/−15%:0.5+/−15%:0.5+/−15%:1.81+/−15%:2+/−15%:0.15+/−15%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−10%:0.5+/−10%:0.5+/−10%:1.5+/−10%:2+/−10%:0.15+/−10% or 1+/−10%:0.5+/−10%:0.5+/−10%:1.81+/−10%:2+/−10%:0.15+/−10%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−5%:0.5+/−5%:0.5+/−5%:1.5+/−5%:2+/−5%:0.15+/−5% or 1+/−5%:0.5+/−5%:0.5+/−5%:1.81+/−5%:2+/−5%:0.15+/−5%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1:0.5:0.5:1.5:2:0.15 or 1:0.5:0.5:1.81:2:0.15.

An exemplary composition can include a weight (wt.) ratio of the leucine amino acid entity, the isoleucine amino acid entity, the valine amino acid entity, the arginine amino acid entity, the glutamine amino acid entity, and the NAC-amino acid entity of 1+/−20%:0.5+/−20%:0.5+/−20%:1.5+/−20%:2+/−20%:0.3+/−20% or 1+/−20%:0.5+/−20%:0.5+/−20%:1.81+/−20%:2+/−20%:0.3+/−20%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−15%:0.5+/−15%:0.5+/−15%:1.5+/−15%:2+/−15%:0.3+/−15% or 1+/−15%:0.5+/−15%:0.5+/−15%:1.81+/−15%:2+/−15%:0.3+/−15%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−10%:0.5+/−10%:0.5+/−10%:1.5+/−10%:2+/−10%:0.3+/−10% or 1+/−10%:0.5+/−10%:0.5+/−10%:1.81+/−10%:2+/−10%:0.3+/−10%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−5%:0.5+/−5%:0.5+/−5%:1.5+/−5%:2+/−5%:0.3+/−5% or 1+/−5%:0.5+/−5%:0.5+/−5%:1.81+/−5%:2+/−5%:0.3+/−5%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1:0.5:0.5:1.5:2:0.3 or 1:0.5:0.5:1.81:2:0.3.

An exemplary composition can include a weight (wt.) ratio of the leucine amino acid entity, the isoleucine amino acid entity, the valine amino acid entity, the arginine amino acid entity, the glutamine amino acid entity, and the NAC-amino acid entity of 1+/−20%:0.5+/−20%:0.5+/−20%:0.75+/−20%:2+/−20%:0.15+/−20% or 1+/−20%:0.5+/−20%:0.5+/−20%:0.905+/−20%:2+/−20%:0.15+/−20%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−15%:0.5+/−15%:0.5+/−15%:0.75+/−15%:2+/−15%:0.15+/−15% or 1+/−15%:0.5+/−15%:0.5+/−15%:0.905+/−15%:2+/−15%:0.15+/−15%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−10%:0.5+/−10%:0.5+/−10%:0.75+/−10%:2+/−10%:0.15+/−10% or 1+/−10%:0.5+/−10%:0.5+/−10%:0.905+/−10%:2+/−10%:0.15+/−10%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−5%:0.5+/−5%:0.5+/−5%:0.75+/−5%:2+/−5%:0.15+/−5% or 1+/−5%:0.5+/−5%:0.5+/−5%:0.905+/−5%:2+/−5%:0.15+/−5%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1:0.5:0.5:0.75:2:0.15 or 1:0.5:0.5:0.905:2:0.15.

An exemplary composition can include a weight (wt.) ratio of the leucine amino acid entity, the isoleucine amino acid entity, the valine amino acid entity, the arginine amino acid entity, the glutamine amino acid entity, and the NAC-amino acid entity of 1+/−20%:0.5+/−20%:0.5+/−20%:0.75+/−20%:2+/−20%:0.3+/−20% or 1+/−20%:0.5+/−20%:0.5+/−20%:0.905+/−20%:2+/−20%:0.3+/−20%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−15%:0.5+/−15%:0.5+/−15%:0.75+/−15%:2+/−15%:0.3+/−15% or 1+/−15%:0.5+/−15%:0.5+/−15%:0.905+/−15%:2+/−15%:0.3+/−15%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−10%:0.5+/−10%:0.5+/−10%:0.75+/−10%:2+/−10%:0.3+/−10% or 1+/−10%:0.5+/−10%:0.5+/−10%:0.905+/−10%:2+/−10%:0.3+/−10%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−5%:0.5+/−5%:0.5+/−5%:0.75+/−5%:2+/−5%:0.3+/−5% or 1+/−5%:0.5+/−5%:0.5+/−5%:0.905+/−5%:2+/−5%:0.3+/−5%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1:0.5:0.5:0.75:2:0.3 or 1:0.5:0.5:0.905:2:0.3.

An exemplary composition can include a weight (wt.) ratio of the leucine amino acid entity, the isoleucine amino acid entity, the valine amino acid entity, the arginine amino acid entity, the glutamine amino acid entity, and the NAC-amino acid entity of 1+/−20%:0.5+/−20%:0.25+/−20%:0.75+/−20%:1+/−20%:0.225+/−20% or 1+/−20%:0.5+/−20%:0.25+/−20%:0.905+/−20%:1+/−20%:0.225+/−20%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−15%:0.5+/−15%:0.25+/−15%:0.75+/−15%:1+/−15%:0.225+/−15% or 1+/−15%:0.5+/−15%:0.25+/−15%:0.905+/−15%:1+/−15%:0.225+/−15%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−10%:0.5+/−10%:0.25+/−10%:0.75+/−10%:1+/−10%:0.225+/−10% or 1+/−10%:0.5+/−10%:0.25+/−10%:0.905+/−10%:1+/−10%:0.225+/−10%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−5%:0.5+/−5%:0.25+/−5%:0.75+/−5%:1+/−5%:0.225+/−5% or 1+/−5%:0.5+/−5%:0.25+/−5%:0.905+/−5%:1+/−5%:0.225+/−5%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1:0.5:0.25:0.75:1:0.225 or 1:0.5:0.25:0.905:1:0.225.

An exemplary composition comprising amino acid entities can include a weight (wt.) ratio of the leucine amino acid entity, the isoleucine amino acid entity, the valine amino acid entity, the arginine amino acid entity, the glutamine amino acid entity, the NAC-amino acid entity, and the serine amino acid entity of 1+/−20%:0.5+/−20%:0.25+/−20%:0.75+/−20%:1+/−20%:0.225+/−20%:1.5+/−20% or 1+/−20%:0.5+/−20%:0.25+/−20%:0.905+/−20%:1+/−20%:0.225+/−20%:1.5+/−20%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1+/−15%:0.5+/−15%:0.25+/−15%:0.75+/−15%:1+/−15%:0.225+/−15%:1.5+/−15% or 1+/−15%:0.5+/−15%:0.25+/−15%:0.905+/−15%:1+/−15%:0.225+/−15%:1.5+/−15%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1+/−10%:0.5+/−10%:0.25+/−10%:0.75+/−10%:1+/−10%:0.225+/−10%:1.5+/−10% or 1+/−10%:0.5+/−10%:0.25+/−10%:0.905+/−10%:1+/−10%:0.225+/−10%:1.5+/−10%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1+/−5%:0.5+/−5%:0.25+/−5%:0.75+/−5%:1+/−5%:0.225+/−5%:1.5+/−5% or 1+/−5%:0.5+/−5%:0.25+/−5%:0.905+/−5%:1+/−5%:0.225+/−5%:1.5+/−5%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1:0.5:0.25:0.75:1:0.225:1.5 or 1:0.5:0.25:0.905:1:0.225:1.5.

An exemplary composition can include a weight (wt.) ratio of the leucine amino acid entity, the isoleucine amino acid entity, the valine amino acid entity, the arginine amino acid entity, the glutamine amino acid entity, the NAC-amino acid entity, and the serine amino acid entity of 1+/−20%:0.5+/−20%:0.25+/−20%:0.75+/−20%:1+/−20%:0.225+/−20%:1.667+/−20% or 1+/−20%:0.5+/−20%:0.25+/−20%:0.905+/−20%:1+/−20%:0.225+/−20%:1.667+/−20%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1+/−15%:0.5+/−15%:0.25+/−15%:0.75+/−15%:1+/−15%:0.225+/−15%:1.667+/−15% or 1+/−15%:0.5+/−15%:0.25+/−15%:0.905+/−15%:1+/−15%:0.225+/−15%:1.667+/−15%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1+/−10%:0.5+/−10%:0.25+/−10%:0.75+/−10%:1+/−10%:0.225+/−10%:1.667+/−10% or 1+/−10%:0.5+/−10%:0.25+/−10%:0.905+/−10%:1+/−10%:0.225+/−10%:1.667+/−10%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1+/−5%:0.5+/−5%:0.25+/−5%:0.75+/−5%:1+/−5%:0.225+/−5%:1.667+/−5% or 1+/−5%:0.5+/−5%:0.25+/−5%:0.905+/−5%:1+/−5%:0.225+/−5%:1.667+/−5%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1:0.5:0.25:0.75:1:0.225:1.667 or 1:0.5:0.25:0.905:1:0.225:1.667.

In some embodiments, the composition includes 10 wt. %+/−15% to 30 wt. %+/−15% of an leucine amino acid entity, 5 wt. %+/−15% to 15 wt. %+/−15% of a isoleucine amino acid entity, 5 wt. %+/−15% to 15 wt. %+/−15% of a valine amino acid entity, 15 wt. %+/−15% to 40 wt. %+/−15% of an arginine amino acid entity, 20 wt. %+/−15% to 50 wt. %+/−15% of a glutamine amino acid entity, and 1 wt. %+/−15% to 8 wt. %+/−15% of a NAC entity.

In some embodiments, the composition includes 10 wt. %+/−15% to 30 wt. %+/−15% of an leucine amino acid entity. In some embodiments, the composition includes 5 wt. %+/−15% to 15 wt. %+/−15% of a isoleucine amino acid entity. In some embodiments, the composition includes 5 wt. %+/−15% to 15 wt. %+/−15% of a valine amino acid entity. In some embodiments, the composition includes 15 wt. %+/−15% to 40 wt. %+/−15% of an arginine amino acid entity. In some embodiments, the composition includes 20 wt. %+/−15% to 50 wt. %+/−15% of a glutamine amino acid entity. In some embodiments, the composition includes 1 wt. %+/−15% to 8 wt. %+/−15% of a NAC entity.

In some embodiments, the composition includes 16 wt. %+/−15% to 18 wt. %+/−15% of an leucine amino acid entity, 7 wt. %+/−15% to 9 wt. %+/−15% of a isoleucine amino acid entity, 7 wt. %+/−15% to 9 wt. %+/−15% of a valine amino acid entity, 28 wt. %+/−15% to 32 wt. %+/−15% of an arginine amino acid entity, 31 wt. %+/−15% to 34 wt. %+/−15% of a glutamine amino acid entity, and 1 wt. %+/−15% to 5 wt. %+/−15% of a NAC-entity. In some embodiments, the composition includes 16 wt. %+/−15% to 18 wt. %+/−15% of an leucine amino acid entity. In some embodiments, the composition includes 7 wt. %+/−15% to 9 wt. %+/−15% of a isoleucine amino acid entity. In some embodiments, the composition includes 7 wt. %+/−15% to 9 wt. %+/−15% of a valine amino acid entity. In some embodiments, the composition includes 28 wt. %+/−15% to 32 wt. %+/−15% of an arginine amino acid entity. In some embodiments, the composition includes 31 wt. %+/−15% to 34 wt. %+/−15% of a glutamine amino acid entity. In some embodiments, the composition includes 1 wt. %+/−15% to 5 wt. %+/−15% of a NAC-entity.

In some embodiments, the composition includes 16.8 wt. %+/−15% of an leucine amino acid entity, 8.4 wt. %+/−15% of a isoleucine amino acid entity, 8.4 wt. %+/−15% of a valine amino acid entity, 30.4 wt. %+/−15% of an arginine amino acid entity, 33.6 wt. %+/−15% of a glutamine amino acid entity, and 2.5 wt. %+/−15% of a NAC-entity.

iii. Relationships of Amino Acid Entities

In some embodiments, the composition (e.g., the Active Moiety) has one or more of the following properties:

a) a wt. % of the Q-amino acid entity in the composition is greater than the wt. % of the R-amino acid entity;
b) the wt. % of the Q-amino acid entity in the composition is greater than the wt. % of the L-amino acid entity;
c) the wt. % of the R-amino acid entity in the composition is greater than the wt. % of the L-amino acid entity; or
d) a combination of two or three of (a)-(c).

In some embodiments, the wt. % of the glutamine amino acid entity in the composition is greater than the wt. % of the arginine amino acid entity, e.g., the wt. % of the glutamine amino acid entity in the composition is at least 5% greater than the wt. % of the arginine amino acid entity, e.g., the wt. % of the glutamine amino acid entity is at least 10% or 25% greater than the wt. % of the arginine amino acid entity.

In some embodiments, the wt. % of the glutamine amino acid entity in the composition is greater than the wt. % of the leucine amino acid entity, e.g., the wt. % of the glutamine amino acid entity in the composition is at least 20% greater than the wt. % of the leucine amino acid entity, e.g., the wt. % of the glutamine amino acid entity in the composition is at least 25% 50% greater than the wt. % of the leucine amino acid entity.

In some embodiments, the wt. % of the arginine amino acid entity in the composition is greater than the wt. % of the leucine amino acid entity, e.g., the wt. % of the arginine amino acid entity in the composition is at least 10% greater than the wt. % of the leucine amino acid entity, e.g., the wt. % of the arginine amino acid entity in the composition is at least 15% or 30% greater than the wt. % of the leucine amino acid entity.

In some embodiments, the wt. % of the leucine amino acid entity in the composition is greater than the wt. % of the isoleucine amino acid entity in the composition, e.g., the wt. % of the leucine amino acid entity in the composition is at least 25 wt. % greater than the wt. % of the isoleucine amino acid entity in the composition.

In some embodiments, the wt. % of the leucine amino acid entity in the composition is greater than the wt. % of the valine amino acid entity in the composition, e.g., the wt. % of the leucine amino acid entity in the composition is at least 25 wt. % greater than the wt. % of the valine amino acid entity in the composition.

In some embodiments, the wt. % of the arginine amino acid entity, the glutamine amino acid entity, and the NAC entity is at least: 50 wt. % or 70 wt. % of the amino acid entities in the composition, but not more than 90 wt. % of the amino acid entities in the composition.

In some embodiments, the wt. % of the NAC entity is at least: 1 wt. % or 2 wt. % of the amino acid entity components or total components in the composition, but not more than 10 wt. % or more of the amino acid entity components or total components in the composition.

In some embodiments, the isoleucine amino acid entity, and the valine amino acid entity in combination is at least: 15 wt. %, or 20 wt. % of the amino acid entity components or total components in the composition, but not more than: 50 wt. % of the amino acid entity components or total components in the composition;

In some embodiments, the glutamine amino acid entity, and the NAC entity is at least: 40 wt. % or 50 wt. % of the amino acid entity components or total components in the composition, but not more than 90 wt. % of the amino acid entity components or total components in the composition.

In some embodiments, the composition (e.g., the Active Moiety) further comprises an serine amino acid entity, e.g., the serine amino acid entity is present at a higher amount than any other amino acid entity component in the composition. In some embodiments, the wt. % of the serine amino acid entity is at least 20 wt. % or more of the amino acid entities or total components in the composition.

iv. Amino Acid Molecules to Exclude or Limit from the Composition

In some embodiments, the composition does not comprise a peptide of more than 20 amino acid residues in length (e.g., protein supplement) chosen from or derived from one, two, three, four, five, or more (e.g., all) of egg white protein, soy protein, casein, hemp protein, pea protein, or brown rice protein, or if the peptide is present, the peptide is present at less than: 10 weight (wt.) 5 wt. %, 1 wt. %, 0.1 wt. %, 0.05 wt. %, 0.01 wt. %, of the total wt. of non-amino acid entity protein components or total components in the composition (e.g., in dry form).

In some embodiments, the composition comprises a combination of 3 to 19, 3 to 15, or 3 to 10 different amino acid entities; e.g., the combination comprises at least: 42 wt. %, 75 wt. %, or 90 wt. % of the total wt. % of amino acid entity components or total components in the composition (e.g., in dry form).

In some embodiments, dipeptides or salts thereof or tripeptides or salts thereof are present at less than: 10 wt. %, 0.5 wt. %, 0.1 wt. %, 0.05 wt. %, 0.01 wt. %, 0.001 wt. %, or less of the total wt. of amino acid entity components or total components in the composition (e.g., in dry form).

In some embodiments, at least 50%, 60%, 70%, or more of the total grams of amino acid entity components in the composition (e.g., in dry form) are from one, two, three, four, five, seven, eight, nine, or more (e.g., all) of (a)-(j).

In some embodiments, at least: 50%, 60%, 70%, or more of the calories from amino acid entity components or total components in the composition (e.g., in dry form) are from one, two, three, four, five, seven, eight, nine, or more (e.g., all) of (a)-(j).

In some embodiments, a carbohydrate (e.g., one, two, three, four, five, six, seven, eight, nine, 10, 11, 12, 13, 14, 15, 16, 17, or 18 of dextrose, maltodextrose, sucrose, dextrin, fructose, galactose, glucose, glycogen, high fructose corn syrup, honey, inositol, invert sugar, lactose, levulose, maltose, molasses, sugarcane, or xylose) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a vitamin (e.g., one, two, three, four, five, six, or seven of vitamin B 1, vitamin B2, vitamin B3, vitamin B6, vitamin B12, vitamin C, or vitamin D) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, one or both of nitrate or nitrite are absent from the composition, or if present, are present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, 4-hydroxyisoleucine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a probiotic (e.g., a Bacillus probiotic) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, phenylacetate is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, gelatin (e.g., a gelatin capsule) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, one, two, or three of S-allyl cysteine, S-allylmercaptocysteine, or fructosyl-arginine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

### Uses, e.g., Methods of Treatment

The composition of the invention as described herein (e.g., the Active Moiety) can be administered to improve or reduce fibrosis, e.g., treat or prevent a fibrotic condition or disorder in a subject. The method includes administering the composition described herein to a subject in need thereof, in an amount sufficient to decrease or inhibit fibrosis in the subject. The composition can be administered to improve tissue repair, e.g., in a patient with a fibrotic condition or disorder.

In some embodiments, the subject has fibrosis or has been diagnosed with a fibrotic condition or disorder. In some embodiments, the subject with a fibrotic condition or disorder is a human. In some embodiments, the subject has not received prior treatment with the composition (e.g., a naïve subject).

The disclosure features a method for improving or reducing fibrosis, comprising administering to a subject in need thereof an effective amount of a composition disclosed herein (e.g., an Active Moiety). The composition can be administered according to a dosage regimen described herein to treat a subject with a fibrotic condition or disorder.

In some embodiments, the composition described herein (e.g., the Active Moiety) is for use as a medicament in treating (e.g., reversing, reducing, ameliorating, or preventing) fibrosis in a subject (e.g., a subject with a fibrotic condition or disorder). In some embodiments, the composition described herein (e.g., the Active Moiety) is for use in the manufacture of a medicament for treating (e.g., reversing, reducing, ameliorating, or preventing) fibrosis in a subject (e.g., a subject with a fibrotic condition or disorder).

In certain embodiments, reducing or treating fibrosis includes reducing one, two, three, four, five or more (e.g., all) of: the formation or deposition of tissue fibrosis; the size, cellularity (e.g., fibroblast or immune cell numbers), composition, or cellular content of a fibrotic lesion; the collagen or hydroxyproline content of a fibrotic lesion; expression or activity of a fibrogenic protein; fibrosis associated with an inflammatory response; or weight loss associated with fibrosis. In some embodiments, reducing fibrosis increases survival of a subject.

Exemplary fibrotic diseases include, but are not limited to, multi-systemic (e.g., systemic sclerosis, multifocal fibrosclerosis, sclerodermatous graft-versus-host disease in bone marrow transplant recipients, nephrogenic systemic fibrosis, or scleroderma) and organ-specific disorders (e.g., fibrosis of the lung, heart, kidney, pancreas, skin, brain, and other organs). In certain embodiments, the fibrotic condition is a fibrotic condition of the lung, a fibrotic condition of the a fibrotic condition of the heart or vasculature, a fibrotic condition of the kidney, a fibrotic condition of the skin, a fibrotic condition of the gastrointestinal tract, a fibrotic condition of the bone marrow or hematopoietic tissue, a fibrotic condition of the nervous system, a fibrotic condition of the eye, or a combination thereof.

In certain embodiments, the fibrotic condition is primary fibrosis. In one embodiment, the fibrotic condition is idiopathic. In other embodiments, the fibrotic condition is associated with (e.g., is secondary to) a disease (e.g., an infectious disease, an inflammatory disease, an autoimmune disease, and/or a connective disease); a toxin; an insult (e.g., an environmental hazard (e.g., asbestos, coal dust, and/or polycyclic aromatic hydrocarbons), cigarette smoking, or a wound); a medical treatment (e.g., surgical incision, chemotherapy, or radiation); or a combination thereof.

In certain embodiments, the fibrotic condition is a fibrotic condition of the lung. In certain embodiments, the fibrotic condition of the lung is chosen from one or more of: pulmonary fibrosis, idiopathic pulmonary fibrosis (IPF), usual interstitial pneumonitis (UIP), interstitial lung disease, cryptogenic fibrosing alveolitis (CFA), bronchiectasis, and scleroderma lung disease. In one embodiment, the fibrosis of the lung is secondary to a disease, a toxin, an insult, a medical treatment, or a combination thereof.

For example, the fibrosis of the lung can be associated with (e.g., secondary to) one or more of: a disease process, such as asbestosis and silicosis; an occupational hazard; an environmental pollutant; cigarette smoking; an autoimmune connective tissue disorders (e.g., rheumatoid arthritis, scleroderma and systemic lupus erythematosus (SLE)); a connective tissue disorder (e.g., sarcoidosis); or an infectious disease (e.g., infection, particularly chronic infection). In one embodiment, the fibrotic condition of the lung is associated with an autoimmune connective tissue disorder (e.g., scleroderma or lupus, e.g., SLE).

In other embodiments, pulmonary fibrosis includes, but is not limited to, pulmonary fibrosis associated with chronic obstructive pulmonary disease (COPD), acute respiratory distress syndrome, scleroderma, pleural fibrosis, chronic asthma, acute lung syndrome, amyloidosis, bronchopulmonary dysplasia, Caplan's disease, Dressler's syndrome, histiocytosis X, idiopathic pulmonary haemosiderosis, lymphangiomyomatosis, mitral valve stenosis, polymyositis, pulmonary edema, pulmonary hypertension (e.g., idiopathic pulmonary hypertension (IPH)), pneumoconiosis, radiotherapy (e.g., radiation induced fibrosis), rheumatoid disease, Shaver's disease, systemic lupus erythematosus, systemic sclerosis, tropical pulmonary eosinophilia, tuberous sclerosis, Weber-Christian disease, Wegener's granulomatosis, Whipple's disease, or exposure to toxins or irritants (e.g., pharmaceutical drugs, such as amiodarone, bleomycin, busulphan, carmustine, chloramphenicol, hexamethonium, methotrexate, methysergide, mitomycin C, nitrofurantoin, penicillamine, peplomycin, or practolol; or inhalation of talc or dust, e.g., coal dust, silica). In certain embodiments, the pulmonary fibrosis is associated with an inflammatory disorder of the lung, e.g., one or both of asthma or COPD.

In certain embodiments, the fibrotic condition is a fibrotic condition of the kidney. In certain embodiments, the fibrotic condition of the kidney is chosen from one or more of: renal fibrosis (e.g., chronic kidney fibrosis), nephropathies associated with one or both of injury or fibrosis (e.g., chronic nephropathies associated with diabetes (e.g., diabetic nephropathy)), lupus, scleroderma of the kidney, glomerular nephritis, focal segmental glomerular sclerosis, IgA nephropathyrenal fibrosis associated with human chronic kidney disease (CKD), chronic progressive nephropathy (CPN), tubulointerstitial fibrosis, ureteral obstruction, chronic uremia, chronic interstitial nephritis, radiation nephropathy, glomerulosclerosis, progressive glomerulonephrosis (PGN), endothelial/thrombotic microangiopathy injury, HIV-associated nephropathy, or fibrosis associated with exposure to a toxin, an irritant, or a chemotherapeutic agent. In one embodiment, the fibrotic condition of the kidney is scleroderma of the kidney. In some embodiments, the fibrotic condition of the kidney is transplant nephropathy, diabetic nephropathy, lupus nephritis, focal segmental glomerulosclerosis (FSGS), endothelial/thrombotic microangiopathy injury, or HIV-associated nephropathy (HIVVAN).

In other embodiments, the fibrotic condition is associated with leprosy or tuberculosis.

In other embodiments, the composition described herein is used to treat a hyperproliferative fibrotic disease, e.g., a non-cancerous fibrotic disease. In one embodiment, the hyperproliferative fibrotic disease is multisystemic or organ-specific. Exemplary hyperproliferative fibrotic diseases include, but are not limited to, multisystemic (e.g., systemic sclerosis, multifocal fibrosclerosis, sclerodermatous graft-versus-host disease in bone marrow transplant recipients, nephrogenic systemic fibrosis, or scleroderma), and organ-specific disorders (e.g., fibrosis of the eye, lung, heart, kidney, pancreas, skin, and other organs).

In certain embodiments, the fibrotic condition is a fibrotic condition of the heart. In certain embodiments, the fibrotic condition of the heart is myocardial fibrosis (e.g., myocardial fibrosis associated with radiation myocarditis, a surgical procedure complication (e.g., myocardial post-operative fibrosis); infectious diseases (e.g., Chagas disease, bacterial, trichinosis, or fungal myocarditis)); granulomatous; metabolic storage disorders (e.g., cardiomyopathy, hemochromatosis); developmental disorders (e.g, endocardial fibroelastosis); arteriosclerotic, or exposure to toxins or irritants (e.g., drug induced cardiomyopathy, drug induced cardiotoxicity, alcoholic cardiomyopathy, cobalt poisoning or exposure). In certain embodiments, the myocardial fibrosis is associated with an inflammatory disorder of cardiac tissue (e.g., myocardial sarcoidosis). In some embodiments, the fibrotic condition is a fibrotic condition associated with a myocardial infarction. In some embodiments, the fibrotic condition is a fibrotic condition associated with congestive heart failure.

In some embodiments, the fibrotic condition is associated with an autoimmune disease selected from scleroderma or lupus, e.g., systemic lupus erythematosus.

In some embodiments, the fibrotic condition is systemic. In some embodiments, the fibrotic condition is systemic sclerosis (e.g., limited systemic sclerosis, diffuse systemic sclerosis, or systemic sclerosis sine scleroderma), nephrogenic systemic fibrosis, cystic fibrosis, chronic graft vs. host disease, or atherosclerosis.

In some embodiments, the fibrotic condition is scleroderma. In some embodiments, the scleroderma is localized, e.g., morphea or linear scleroderma. In some embodiments, the condition is a systemic sclerosis, e.g., limited systemic sclerosis, diffuse systemic sclerosis, or systemic sclerosis sine scleroderma.

In other embodiment, the fibrotic condition affects a tissue chosen from one or more of: tendon, cartilage, skin (e.g., skin epidermis or endodermis), cardiac tissue, vascular tissue (e.g., artery, vein), pancreatic tissue, lung tissue, kidney tissue, uterine tissue, ovarian tissue, neural tissue, testicular tissue, peritoneal tissue, colon, small intestine, biliary tract, gut, bone marrow, hematopoietic tissue, or eye (e.g., retinal) tissue.

In some embodiments, the fibrotic condition is a fibrotic condition of the eye. In some embodiments, the fibrotic condition is glaucoma, macular degeneration (e.g., age-related macular degeneration), macular edema (e.g., diabetic macular edema), retinopathy (e.g., diabetic retinopathy), or dry eye disease.

In certain embodiments, the fibrotic condition is a fibrotic condition of the skin. In certain embodiments, the fibrotic condition of the skin is chosen from one or more of: skin fibrosis (e.g., hypertrophic scarring, keloid), scleroderma, nephrogenic systemic fibrosis (e.g., resulting after exposure to gadolinium (which is frequently used as a contrast substance for MRIs) in patients with severe kidney failure), and keloid.

In certain embodiments, the fibrotic condition is a fibrotic condition of the gastrointestinal tract. In certain embodiments, the fibrotic condition is chosen from one or more of: fibrosis associated with scleroderma; radiation induced gut fibrosis; fibrosis associated with a foregut inflammatory disorder (e.g., Barrett's esophagus or chronic gastritis), and/or fibrosis associated with a hindgut inflammatory disorder (e.g., inflammatory bowel disease (IBD), ulcerative colitis, or Crohn's disease). In some embodiments, the fibrotic condition of the gastrointestinal tract is fibrosis associated with scleroderma.

In one embodiment, the fibrotic condition is a chronic fibrotic condition or disorder. In certain embodiments, the fibrotic condition is associated with an inflammatory condition or disorder.

In some embodiments, the fibrotic and/or inflammatory condition is osteomyelitis, e.g., chronic osteomyelitis.

In some embodiments, the fibrotic condition is an amyloidosis. In certain embodiments, the amyloidosis is associated with chronic osteomyelitis.

In some embodiments, the fibrotic condition or disorder is a fibrotic condition or disorder of the liver. In certain embodiments, the fibrotic condition of the liver is chosen from: non-alcoholic fatty liver (NAFL), non-alcoholic fatty liver disease (NAFLD), non-alcoholic steatohepatitis (NASH), alcoholic fatty liver disease (AFLD), or alcoholic steatohepatitis (ASH). In some embodiments, the fibrotic condition of the liver is chosen from: cirrhosis, cholestatic liver disease (e.g., primary biliary cirrhosis (PBC)), biliary duct injury, biliary fibrosis, or cholangiopathies.

In some embodiments, the fibrotic condition or disorder is not a liver fibrotic condition or disorder. In some embodiments, the fibrotic condition or disorder is not a muscle fibrotic condition or disorder.

### Dosage Regimens

The composition (e.g., the Active Moiety) can be administered according to a dosage regimen described herein to reduce or treat fibrosis. For example, the composition may be administered to the subject for a treatment period of, e.g., two weeks, three weeks, four weeks, five weeks, six weeks, seven weeks, eight weeks, nine weeks, 10 weeks, 11 weeks, 12 weeks, 13 weeks, 14 weeks, 15 weeks, 16 weeks, or longer at a dose of 2 g+/−20% g daily to 90 g+/−20% g daily (e.g., 72 g+/−20% total amino acid entities daily).

In some embodiments, the composition can be provided to a subject with a fibrotic condition or disorder in either a single or multiple dosage regimen. In some embodiments, a dose is administered twice daily, three times daily, four times daily, five times daily, six times daily, seven times daily, or more. In certain embodiments, the composition is administered one, two, or three times daily. In some embodiments, the composition is administered for at least 2 days, 3 days, 4 days, 5 days, 6 days, 7 days, or 2 weeks. In some embodiments, the composition is administered chronically (e.g., more than 30 days, e.g., 31 days, 40 days, 50 days, 60 days, 3 months, 6 months, 9 months, one year, two years, or three years).

In some embodiments, the composition is administered prior to a meal. In other embodiments, the composition is administered concurrent with a meal. In other embodiments, the composition is administered following a meal.

The composition can be administered every 2 hours, every 3 hours, every 4 hours, every 5 hours, every 6 hours, every 7 hours, every 8 hours, every 9 hours, or every 10 hours to improve or reduce fibrosis in a subject (e.g., a subject having a fibrotic condition or disorder).

In some embodiments, the composition comprises four stick packs, e.g., each stick pack comprising 25%+/−15% of the quantity of each amino acid entity included in the composition described herein. In certain embodiments, four stick packs are administered three times daily. In some embodiments, the composition comprises three stick packs, e.g., each stick pack comprising 33.3%+/−15% of the quantity of each amino acid entity included in the composition described herein. In certain embodiments, three stick packs are administered three times daily.

In some embodiments, the composition is administered at a dose of about 2 g+/−20% to 50 g+/−20% total amino acid entities, e.g., once per day, twice per day, three times per day, four times per day, five times per day, or six times per day (e.g., three times per day). In certain embodiments, the composition is administered at a dose of 2 g+/−20% to 10 g+/−20% total amino acid entities three times daily, e.g., 8 g+/−20% or 10 g+/−20% total amino acid entities three times daily. In certain embodiments, the composition is administered at a dose of 10 g+/−20% to 20 g+/−20% total amino acid entities three times daily, e.g., 11 g+/−20%, 12 g+/−20%, 15 g+/−20%, 16 g+/−20%, or 20 g+/−20% total amino acid entities three times daily. In certain embodiments, the composition is administered at a dose of 20 g+/−20% to 30 g+/−20% total amino acid entities three times daily, e.g., 21 g+/−20%, 22 g+/−20%, 23 g+/−20%, or 24 g+/−20% total amino acid entities three times daily.

### Production of Active Moiety and Pharmaceutical Compositions

The present disclosure features a method of manufacturing or making a composition (e.g., an Active Moiety) of the foregoing invention. Amino acid entities used to make the compositions may be agglomerated, and/or instantized to aid in dispersal and/or solubilization. The compositions may be made using amino acid entities from the following sources, or other sources may used: e.g., FUSI-BCAA™ Instantized Blend (L-Leucine, L-Isoleucine and L-Valine in 2:1:1 weight ratio), instantized L-Leucine, and other acids may be obtained from Ajinomoto Co., Inc. Pharma. grade amino acid entity raw materials may be used in the manufacture of pharmaceutical amino acid entity products. Food (or supplement) grade amino acid entity raw materials may be used in the manufacture of dietary amino acid entity products.

To produce the compositions of the instant disclosure, the following general steps may be used: the starting materials (individual amino acid entities and excipients) may be blended in a blending unit, followed by verification of blend uniformity and amino acid entity content, and filling of the blended powder into stick packs or other unit dosage form. The content of stick packs or other unit dosage forms may be dispersed in water at time of use for oral administration.

Food supplement and medical nutrition compositions of the invention will be in a form suitable for oral administration.

When combining raw materials, e.g., pharmaceutical grade amino acid entities and/or excipients, into a composition, contaminants may be present in the composition. A composition meets a standard for level of contamination when the composition does not substantially comprise (e.g., comprises less than 10, 9, 8, 7, 6, 5, 4, 3, 2, 1, 0.1, 0.01, or 0.001% (w/w)) a contaminant. In some embodiments, a composition described in a method herein does not comprise a contaminant. Contaminants include any substance that is not deliberately present in the composition (for example, pharmaceutical grade amino acid entities and excipients, e.g., oral administration components, may be deliberately present) or any substance that has a negative effect on a product quality parameter of the composition (e.g., side effects in a subject, decreased potency, decreased stability/shelf life, discoloration, odor, bad taste, bad texture/mouthfeel, or increased segregation of components of the composition). In some embodiments, contaminants include microbes, endotoxins, metals, or a combination thereof. In some embodiments, the level of contamination, e.g., by metals, lecithin, choline, endotoxin, microbes, or other contaminants (e.g., contaminants from raw materials) of each portion of a composition is below the level permitted in food.

### Excipients

The amino acid compositions of the present disclosure may be compounded or formulated with one or more excipients. Non-limiting examples of suitable excipients include a tastant, a flavorant, a buffering agent, a preservative, a stabilizer, a binder, a compaction agent, a lubricant, a dispersion enhancer, a disintegration agent, a flavoring agent, a sweetener, and a coloring agent.

In some embodiments, the excipient comprises a buffering agent. Non-limiting examples of suitable buffering agents include citric acid, sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In some embodiments, the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the composition comprises a dispersion enhancer as an excipient. Non-limiting examples of suitable dispersants include starch, alginic acid, polyvinylpyrrolidones, guar gum, kaolin, xanthan gum, bentonite, purified wood cellulose, sodium starch glycolate, isoamorphous silicate, and microcrystalline cellulose as high HLB emulsifier surfactants.

In some embodiments, the composition comprises a disintegrant as an excipient. In some embodiments, the disintegrant is a non-effervescent disintegrant. Non-limiting examples of suitable non-effervescent disintegrants include starches such as corn starch, potato starch, pregelatinized and modified starches thereof, sweeteners, clays, such as bentonite, microcrystalline cellulose, alginates, sodium starch glycolate, gums such as agar, guar, locust bean, karaya, pecitin, and tragacanth. In some embodiments, the disintegrant is an effervescent disintegrant. Non-limiting examples of suitable effervescent disintegrants include sodium bicarbonate in combination with citric acid, and sodium bicarbonate in combination with tartaric acid.

In some embodiments, the excipient comprises a flavoring agent. Flavoring agents can be chosen from synthetic flavor oils and flavoring aromatics; natural oils; extracts from plants, leaves, flowers, and fruits; and combinations thereof. In some embodiments, the flavoring agent is selected from cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments, the excipient comprises a sweetener. Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; and sugar alcohols such as sorbitol, mannitol, xylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof. In some embodiments, the composition comprises a coloring agent. Non-limiting examples of suitable color agents include food, drug and cosmetic colors (FD&C), drug and cosmetic colors (D&C), and external drug and cosmetic colors (Ext. D&C). The coloring agents can be used as dyes or their corresponding lakes.

Particular excipients may include one or more of: citric acid, lecithin, (e.g. Alcolec F100), sweeteners (e.g. sucralose, sucralose micronized NF, acesulfame potassium (e.g. Ace-K)), a dispersion enhancer (e.g. xanthan gum (e.g. Ticaxan Rapid-3)), flavorings (e.g. vanilla custard #4306, Nat Orange WONF #1326, lime 865.0032U, and lemon 862.2169U), a bitterness masking agent (e.g. 936.2160U), and natural or artificial colorings (e.g. FD&C Yellow 6). Exemplary ingredient contents for each stick pack are shown in Table 7.

TABLE 7
Ingredient contents in each stick pack.

| INGREDIENT | GRADE | FUNCTION | SOURCE; COMMENT |
|---|---|---|---|
| Amino Acids | USP | Active | Various sources; |
| | | Pharmaceutical | Non-instantized |
| | | Ingredient (API) | form (MFG scale) |
| Citric Acid | USP | pH, Flavor | Spectrum Chems; |
| | | | f(volume) ≤ 1.0% w/v |
| Acesulfame K | NF | Sweetness | Spectrum Chems; |
| | | (rapid onset) | Target 1 |
| | | | Sweetener |
| Sucralose | NF | Sweetness | Spectrum Chems; |
| | | (slow onset) | WHO |
| | | | ADI ≤ 15 mg/kg |
| Lecithin | FCC | Wetting Agent | American Lecithin |
| (Alecolec | | | Company |
| F100) | | | |
| Xanthan Gum | FCC | Stabilizer/ | TIC Gums; |
| | | Thickener | f(volume) ≤ |
| | | | 0.5% w/v |
| Vanilla Custard | GRAS | Taste/Aroma | David Michael; Mask |
| (Art) | | | sulfur |
| Orange (Natural | GRAS | 1° flavor | David Michael; Citrus |
| and WONF) | | | profile matches low |
| | | | pH |
| Lime (Natural | GRAS | 2° flavor | FONA; Single flavor |
| and WONF) | | | supplier |
| Lemon (Natural | GRAS | 2° flavor | FONA; Single flavor |
| and artificial) | | | supplier |
| Taste Modifier | GRAS | Bitterness | FONA; Useful at low |
| | | masking | volume |
| FD&C Yellow | USP | Color | Sensient; Match flavor |
| No. 6 | | | profile |

In another embodiment, excipients are limited to citric acid, a sweetener (e.g., sucralose), xanthan gum, an aroma agent (e.g., vanilla custard #4036), a flavoring agent (e.g., Nat orange WONF #1362), and a coloring agent (e.g., FD&C Yellow 6), e.g., the excipient specifically excludes lecithin (Table 8).

TABLE 8
Exemplary contents in each stick pack.

| INGREDIENT | GRADE | FUNCTION |
|---|---|---|
| Amino Acids | USP | Active Pharmaceutical |
| | | Ingredient (API) |
| Citric Acid | USP | pH, Flavor |
| Sucralose | NF | Sweetness (slow onset) |
| Xanthan Gum | FCC | Stabilizer/Thickener |
| Vanilla Custard (Art) | GRAS | Aroma |
| Orange (Nat + WONF) | GRAS | 1° flavor |
| FD&C Yellow No. 6 | USP | Color |

### Dietary Compositions

The composition (e.g., the Active Moiety) including amino acid entities can be formulated and used as a dietary composition, e.g., chosen from a medical food, a functional food, or a supplement. In such an embodiment, the raw materials and final product should meet the standards of a food product.

The composition of any of the aspects and embodiments disclosed herein can be for use as a dietary composition, e.g., chosen from a medical food, a functional food, or a supplement. In some embodiments, the dietary composition is for use in a method, comprising administering the composition to a subject. The composition can be for use in a dietary composition for the purpose of improving or reducing fibrosis.

In some embodiments, the dietary composition is chosen from a medical food, a functional food, or a supplement. In some embodiments, the composition is in the form of a nutritional supplement, a dietary formulation, a functional food, a medical food, a food, or a beverage comprising a composition described herein. In some embodiments, the nutritional supplement, the dietary formulation, the functional food, the medical food, the food, or the beverage comprising a composition described herein for use in the management of fibrosis (e.g., in a subject with a fibrotic condition or disorder).

The present disclosure features a method of improving fibrosis comprising administering to a subject an effective amount of a dietary composition described herein.

The present disclosure features a method of providing nutritional support or supplementation to a subject with fibrosis (e.g., a subject with a fibrotic condition or disorder), comprising administering to the subject an effective amount of a composition described herein.

The present disclosure features a method of providing nutritional support or supplementation that aids in the management of fibrosis (e.g., a fibrotic condition or disorder), comprising administering to a subject in need thereof an effective amount of a composition described herein.

In some embodiments, the subject has or has been diagnosed with a fibrotic condition or disorder. In other embodiments, the subject does not have a fibrotic condition or disorder.

Additionally, the compositions can be used in methods of dietary management of a subject (e.g., a subject without fibrosis).

In some embodiments, the subject has a lung fibrotic condition or disorder. In some embodiments, the subject has a heart or vasculature fibrotic condition or disorder. In some embodiments, the subject has a kidney fibrotic condition or disorder. In some embodiments, the subject has a pancreas fibrotic condition or disorder. In some embodiments, the subject has a skin fibrotic condition or disorder. In some embodiments, the subject has a gastrointestinal fibrotic condition or disorder. In some embodiments, the subject has a bone marrow or hematopoietic tissue fibrotic condition or disorder. In some embodiments, the subject has a nervous system fibrotic condition or disorder. In some embodiments, the subject has an eye fibrotic condition or disorder.

### Biomarkers

Any of the methods disclosed herein can include evaluating or monitoring the effectiveness of administering a composition of the invention as described herein (e.g., the Active Moiety) to a subject with fibrosis (e.g., a subject with a fibrotic condition or disorder). The method includes acquiring a value of effectiveness to the composition, such that the value is indicative of the effectiveness of the therapy.

In some embodiments, the subject exhibits increased levels of proC3, e.g., relative to a healthy subject without fibrosis. In some embodiments, the subject exhibits increased levels of ALT, e.g., relative to a healthy subject without fibrosis. In some embodiments, the subject exhibits increased levels of AST, e.g., relative to a healthy subject without fibrosis. In some embodiments, the subject exhibits increased levels of TIMP (e.g., TIMP1 or TIMP2), e.g., relative to a healthy subject without fibrosis. In some embodiments, the subject exhibits increased levels of Col1a1, e.g., relative to a healthy subject without fibrosis. In some embodiments, the subject exhibits increased levels of Acta2, e.g., relative to a healthy subject without fibrosis. In some embodiments, the subject exhibits increased levels of Hsp47, e.g., relative to a healthy subject without fibrosis. In some embodiments, the subject exhibits increased levels of hydroxyproline, e.g., relative to a healthy subject without fibrosis.

In some embodiments, administration of the composition (e.g., the Active Moiety) at a dosage regimen described herein to the subject reduces the level or activity of one, two, three, four, five, six, seven, eight, nine, ten, 11, 12, 13, 14, 15, 16, or more (e.g., all) of the following: (a) N-terminal fragment of type III collagen (proC3); (b) a tissue inhibitor of metalloproteinase (TIMP) protein; e.g., TIMP1 or TIMP2; (c) Col1a1; (d) Acta2; (e) ALT; (f) AST; (g) hydroxyproline; (h) TGF-b; (i) MCP-1; (j) MIP-1; (k) collagen, e.g., type I and III collagen; (l) α-smooth muscle actin (aSMA); (m) PIIINP; (n) Hsp47; (o) procollagen Iα1; (p) YKL40; or (q) GROalpha (CXCL1).

### A Method of Evaluating (e.g., Screening)

In another aspect, disclosed herein is a method or assay for evaluating a composition as described herein. The method includes: (a) contacting one or more liver cell types (e.g., one, two, or three of hepatocyte cells, stellate cells, or macrophages, e.g., in a triculture of hepatocyte cells, stellate cells, and macrophages), e.g. separated by a membrane (e.g., a permeable membrane, e.g., a Transwell) in culture (e.g., hepatocyte cells separated by a membrane from one or both of stellate cells and macrophages) with the composition under the conditions described in Example 9; and (b) detecting a level of a fibrotic marker, e.g., one, two, three, or more (e.g., all) of procollagen Iα1, MCP-1, YKL40, or GROalpha (CXCL1)). In some embodiments, a change (e.g., a decrease) in the level of the fibrotic marker (e.g., one, two, three, or more (e.g., all) of procollagen Iα1, MCP-1, YKL40, or GROalpha (CXCL1)) indicates that the composition is suitable for reducing or treating fibrosis. In some embodiments, the composition results in a decrease, e.g., a decrease of at least 10%, 20%, 30%, 40%, 50%, or more in the level of the fibrotic marker (e.g., one, two, three, or more (e.g., all) of procollagen Iα1, MCP-1, YKL40, or GROalpha (CXCL1)), e.g., the decrease indicative that the composition is suitable for reducing or treating fibrosis. In certain embodiments, the composition results in a decrease of one, two, three, or more (e.g., all) of:
(i) a level of procollagen Iα1 (e.g., a decrease in the level of procollagen Iα1 of at least 20%, 30%, 40%, or 50%);
(ii) a level of MCP1 (e.g., a decrease in the level of MCP1 of at least 50%, 60%, 70%, 80%, or 90%);
(iii) a level of YKL40 (e.g., a decrease in the level of YKL40 of at least 70%, 80%, 90%, or 95%); or
(iv) a level of GROalpha (CXCL1) (e.g., a decrease in the level of GROalpha (CXCL1) of at least 15%, 20%, 25%, or 30%).

In some embodiments, the one or more liver cell types (e.g., hepatocyte cells, stellate cells, and macrophages) are present in a co-culture, e.g., liver cell types separated by a membrane (e.g., a permeable membrane, e.g., a Transwell) in culture (e.g., hepatocyte cells separated by a membrane from one or both of stellate cells or macrophages), e.g., in a ratio of hepatocytes to macrophages to stellate cells of about 10:2:1 (e.g., a ratio of about 10:2:1 of hepatocyte cells separated by a membrane (e.g., a permeable membrane, e.g., a Transwell) to stellate cells to macrophages).

In some embodiments, the detection step comprises obtaining a sample, e.g., a culture sample, e.g., a culture sample from a transwell plate as described in Example 9, and measuring the level of the fibrotic marker (e.g., one, two, three, or more (e.g., all) of procollagen Iα1, MCP-1, YKL40, or GROalpha (CXCL1)).

### EXAMPLES

The Examples below are set forth to aid in the understanding of the inventions, but are not intended to, and should not be construed to, limit its scope in any way.

### Example 1. Therapeutic Amino Acid Composition A-1 Treatment Improves Liver Fibrosis in an Animal Model of Chemically Induced Fibrosis

Amino Acid Composition A-1 was tested for its ability to affect liver fibrosis in a model of chemically induced liver fibrosis. A commonly used model of experimental hepatic fibrosis is induced chemically in mice using carbon tetrachloride; CCl₄(Gideon Smith, Animal Models of Cutaneous and Hepatic Fibrosis; Progress in Molecular Biology and Translational Science, Vol. 105, pp. 371-408). CCl₄ causes inflammation, hepatocyte damage, necrosis and fibrosis after 4 weeks of treatment and cirrhosis after 8 weeks. Liver fibrosis induced in mice by carbon tetrachloride (CCl₄) resembles important properties of human liver fibrosis including inflammation, regeneration and fiber formation.

Male BALB/c mice 7 to 8 weeks of age were used for this study. Animals were housed four per cage, kept on a standard 12 hr light cycle and given free access to water and standard mouse chow. Food and water were available ad libitum.

Animals were dosed with 5% CCl₄ or vehicle intraperitoneally (IP) typically 3 days a week for 4 weeks. CCl₄ was formulated weekly. 10 ml/kg of Amino Acid Composition A-1 at 23 mg/ml, 76 mg/ml or 153 mg/ml was dosed by oral gavage twice daily. Animals were weighed twice weekly and blood was collected via retro-orbital sinus once per week for serum. After four weeks, blood was collected for serum isolation and mice were euthanized via cervical dislocation. Two lobes of liver were removed—the left lobe was placed in a tube containing 10% formalin for histopathology, while the right lobe was weighed and placed in a beadbeater tube containing 2.3 mm zirconia beads and 2× volume of 1:100 protease inhibitor (Sigma Aldrich, #P8340). Tissue samples were homogenized for 2 minutes in a beadbeater machine and immediately spun down at 3,000 rpm for 15 minutes at 4° C. Serum was analyzed for ALT/AST levels at weeks 2 and 4. Homogenized liver samples were further evaluated for Hydroxyproline (Hyp) content to identify formation of liver fibrosis.

Hydroxyproline (Week 4)

Hydroxyproline (4-hydroxyproline, Hyp) is a common nonproteinogenic amino acid and is used as an indirect measure of the amount of collagen present, indicative of fibrosis. Hepatic Hyp content levels in CCl₄-treated animals were significantly higher than vehicle treated animals. Data are mean±standard deviation (stdev); “Comp A-1”: Amino Acid Composition A-1; *p<0.05 compared to vehicle control by unpaired T test. Raw data are shown in Table 9.

TABLE 9
Hepatic Hyp content level results

| | Hydroxyproline | | | | |
|---|---|---|---|---|---|
| | Vehicle/ | Vehicle/ | Comp A-1, | Comp A-1, | Comp A-1, |
| | Sham | CCL4 | 23 mg/ml | 76 mg/ml | 153 mg/ml |
| mean | 0.160 | 0.263* | 0.280 | 0.228 | 0.201 |
| stdev | 0.067 | 0.107 | 0.104 | 0.124 | 0.057 |

AST Levels and ALT Levels

Aspartate transaminase (AST) and alanine transaminase (ALT) are commonly measured clinical biomarkers of liver health. Both AST and ALT levels were significantly elevated in CCl₄ administered animals for the entire duration of the study, suggesting that liver damage has occurred. Data are mean±standard deviation (stdev); “Comp A-1”: Amino Acid Composition A-1; p values are compared to vehicle/CCl₄ control; by one-tailed T test; n.s. not significant. Raw data are shown in Tables 29 and 30.

TABLE 10
ALT level results

| | Liver ALT | | | | |
|---|---|---|---|---|---|
| | Vehicle/ | Vehicle/ | Comp A-1, | Comp A-1, | Comp A-1, |
| | Sham | CCL4 | 23 mg/ml | 76 mg/ml | 153 mg/ml |
| mean | 1608.4 | 4153.4 | 3694.9 | 3023.4 | 2992.7 |
| stdev | 1099.5 | 1427.4 | 2106.4 | 1343.8 | 1674.2 |
| | | | n.s. | p < 0.05 | p = 0.0371 |

TABLE 11
AST level results

| | Liver AST | | | | |
|---|---|---|---|---|---|
| | Vehicle/ | Vehicle/ | Comp A-1, | Comp A-1, | Comp A-1, |
| | Sham | CCL4 | 23 mg/ml | 76 mg/ml | 153 mg/ml |
| mean | 155.8 | 933.6 | 879.2 | 554.7 | 680.4 |
| stdev | 69.7 | 237.0 | 527.3 | 336.6 | 431.2 |
| | | | n.s. | p < 0.01 | p = 0.0394 |

Summary

Treatment with Amino Acid Composition A-1 resulted in reduction of chemically-induced fibrosis as indicated by reduced levels of hydroxyproline, a marker for collagen production, and in improvement of clinical biomarkers of liver damage as indicated by reduction in levels of liver enzymes ALT and AST (Tables 12-14).

TABLE 12
Hepatic Hyp content level results: raw data
Hydroxyproline

| | | Comp A-1, | Comp A-1, | Comp A-1, |
|---|---|---|---|---|
| Vehicle/Sham | Vehicle/CCL4 | 23 mg/ml | 76 mg/ml | 153 mg/ml |
| 0.122 | 0.241 | 0.246154 | 0.190323 | 0.248649 |
| 0.277 | 0.318 | 0.529578 | 0.174684 | 0.24 |
| 0.152 | 0.298 | 0.234783 | 0.226549 | 0.18 |
| 0.108 | 0.493 | 0.216393 | 0.169128 | 0.174233 |
| 0.123 | 0.2 | 0.294737 | 0.175887 | 0.133333 |
| 0.108 | 0.196 | 0.22439 | 0.107692 | 0.135758 |
| 0.232 | 0.183 | 0.305512 | 0.212389 | 0.210219 |
| | 0.177 | 0.393064 | 0.316191 | 0.150265 |
| | | 0.272897 | 0.612174 | 0.231293 |
| | | 0.192683 | 0.18018 | 0.308824 |
| | | 0.164341 | 0.218803 | |
| | | | 0.203279 | |
| | | | 0.17971 | |

TABLE 13
ALT level results: raw data
Liver ALT

| | | Comp A-1, | Comp A-1, | Comp A-1, |
|---|---|---|---|---|
| Vehicle/Sham | Vehicle/CCL4 | 23 mg/ml | 76 mg/ml | 153 mg/ml |
| 685.0737 | 4963.448 | 1299.647 | 4325.237 | 2611.524 |
| 2623.343 | 578.7053 | 5069.816 | 4325.237 | 2150.594 |
| 1606.933 | 5235.278 | 5566.202 | 2304.237 | 1866.945 |
| 3805.214 | 2115.138 | 5188.003 | 1051.454 | 696.8924 |
| 779.6234 | 4384.331 | 3828.851 | 1488.746 | 1725.121 |
| 637.7988 | 4207.05 | 330.5123 | 4313.419 | 3722.483 |
| 1417.834 | 5471.652 | 649.6176 | 4112.501 | 5211.641 |
| 1311.466 | 5105.273 | 1441.471 | 2859.717 | 4797.986 |
| | 3462.471 | 5495.29 | 2564.249 | 1216.916 |
| | 4147.957 | 4892.536 | 5318.009 | 1796.033 |
| | 5436.196 | 5329.828 | 2836.079 | 5069.816 |
| | 3852.489 | 5247.097 | 2457.881 | 5046.179 |
| | 5034.36 | | 1346.922 | |

TABLE 14
AST level results: raw data
Liver AST

| | | Comp A-1, | Comp A-1, | Comp A-1, |
|---|---|---|---|---|
| Vehicle/Sham | Vehicle/CCL4 | 23 mg/ml | 76 mg/ml | 153 mg/ml |
| 95.37346 | 908.3081 | 315.7015 | 703.1751 | 508.1721 |
| 57.38585 | 1050.129 | 928.5682 | 720.9027 | 335.9616 |
| 239.7263 | 877.918 | 1389.484 | 371.4167 | 379.0142 |
| 194.1412 | 660.1224 | 1047.596 | 262.5189 | 211.8688 |
| 123.231 | 599.3423 | 589.2123 | 267.5839 | 510.7046 |
| 102.971 | 675.3175 | 181.4787 | 819.6704 | 885.5156 |
| 237.1938 | 1470.525 | 285.3115 | 629.7324 | 1214.742 |
| 196.6737 | 1070.389 | 305.5715 | 414.4693 | 941.2307 |
| | 733.5651 | 1690.853 | 505.6396 | 252.3889 |
| | 976.6858 | 1100.779 | 1485.72 | 297.974 |
| | 1088.116 | 1232.469 | 356.2217 | 1437.602 |
| | 918.4382 | 1483.187 | 406.8718 | 1189.416 |
| | 1108.376 | | 267.5839 | |

### Example 2. Therapeutic Treatment of NAFLD, NASH, and HCC with Amino Acid Composition A-1 in a Pre-Clinical Animal Model

Amino Acid Composition A-1 and Obeticholic acid (6α-ethyl-chenodeoxycholic acid; “OCA”) were tested for their ability to treat NASH in the STAM™ model (Stelic Institute & Co., Tokyo, Japan; Saito K. et al., 2015 Sci Rep 5: 12466). Two additional groups of normal C57BL/6 mice fed standard chow and vehicle treated STAM™ mice were included as controls. All animals receiving treatment or vehicle were treated starting at 6 weeks until 9 weeks of age. Compounds were administered via oral gavage, with a dose volume of 10 ml/kg. Amino Acid Composition A-1 was administered twice daily at a dose of 1500 mg/kg, and OCA was administered once daily at a dose of 30 mg/kg.

STAM™ is a model for non-alcoholic steatohepatitis (NASH) and hepatocellular carcinoma (HCC), developed by SMC Laboratories, Inc. and created by the combination of chemical and dietary interventions using C57BL/6 mice (Saito K. et al., 2015 Sci Rep 5: 12466). Mice are treated with a low dose of streptozotocin at birth and fed a high fat diet starting at 4 weeks. Evidence of fatty liver is present by 5 weeks, followed by NASH by 7 weeks and fibrosis by 9 weeks.

NASH was induced in 53 male mice by a single subcutaneous injection of 200 μg streptozotocin (STZ, Sigma-Aldrich, USA) solution 2 days after birth and feeding with high fat diet (HFD, 57 kcal % fat, Cat #HFD32, CLEA Japan, Japan) after 4 weeks of age.

Amino Acid Composition A-1, OCA and Vehicle (described below) were administered by oral route in a volume of 10 mL/kg. Amino Acid Composition A-1 was solubilized in deionized water to 150 mg/ml (10×). OCA (Advanced ChemBlocks Inc.) was resuspended in 0.5% methylcellulose in water to 3 mg/ml (10×). Amino Acid Composition A-1 was administered at a dose of 1500 mg/kg twice daily (9 am and 7 pm). OCA was administered at a dose of 30 mg/kg once daily (9 am).

Liver samples from mice in Group 2 (Vehicle), 3 (Amino Acid Composition A-1) and 4 (OCA) were used for the following assays. For HE staining, sections were cut from paraffin blocks of liver tissue prefixed in Bouin's solution and stained with Lillie-Mayer's Hematoxylin (Muto Pure Chemicals Co., Ltd., Japan) and eosin solution (Wako Pure Chemical Industries). NAFLD Activity score (NAS) was calculated according to the criteria of Kleiner (Kleiner D. E. et al., Hepatology, 2005; 41:1313).

Study Groups

Group 1: STZ: Ten neonatal STZ-primed mice were fed with a normal diet ad libitum without any treatment until 9 weeks of age.

Group 2: Vehicle: Ten NASH mice were orally administered vehicle (10% phosphate buffered saline, pH 7.2) in a volume of 10 mL/kg twice daily (9 am and 7 pm) from 6 to 9 weeks of age.

Group 3: Amino Acid Composition A-1: Ten NASH mice were orally administered water for irrigation supplemented with Amino Acid Composition A-1 at a dose of 1500 mg/kg twice daily (9 am and 7 pm) from 6 to 9 weeks of age.

Group 4: OCA: Ten NASH mice were orally administered 0.5% methylcellulose supplemented with OCA at a dose of 30 mg/kg once daily (9 am) from 6 to 9 weeks of age.

Group 5: Normal: Ten normal mice were fed with a normal diet ad libitum without any treatment until 9 weeks of age.

Group 6: HFD: Ten normal mice were fed with a high fat diet ad libitum without any treatment until 9 weeks of age.

### Histological Results: HE Staining, NAFLD Activity Score and α-Smooth Muscle Actin Staining

The non-alcoholic fatty liver disease (NAFLD) activity score was assessed via histological analysis and grading of H&E stained liver sections from each animal. This score is the sum of three individual scores that grade the degree of steatosis (0-3), inflammation (0-2), and hepatocyte ballooning (0-2). All tissues were graded using the scoring criteria of Kleiner et al. (Kleiner et al. Hepatology. 2005; 41(6): 1313-21). Results are shown in Table 15. Data are mean±standard deviation (stdev). Normal C57BL/6 mice fed standard chow had a mean score of 0+/−0. Vehicle treated STAM™ mice had a mean score of 4.7+/−0.67. Amino Acid Composition A-1 treated mice had a mean score of 3.1+/−0.74. OCA treated mice had a mean score of 2.9+/−0.74. Both Amino Acid Composition A-1 and OCA were statistically different from vehicle for NAFLD Activity Score when compared using Dunnett's multiple comparisons test (Amino Acid Composition A-1 p=0.0001, OCA p=0.0001).

Similarly, Amino Acid Composition A-1 treated mice showed a mean ballooning score of 0.4+/−0.52, compared to a mean ballooning score for vehicle treated STAM™ mice of 1.6+/−0.52, and a mean ballooning score for OCA treated mice of 0.3+/−0.48. Both Amino Acid Composition A-1 and OCA were statistically different from vehicle for ballooning score when compared using Dunnett's multiple comparisons test (Amino Acid Composition A-1 p=0.0001, OCA p=0.0001). Raw data are shown in Tables 15-18.

TABLE 15
NAFLD Activity Score
NAFLD Activity Score (NAS)

| | | Vehicle- | Amino Acid | |
|---|---|---|---|---|
| | Normal | treated | Composition | |
| | C57BL/6 | STAM | A-1 treated | OCA treated |
| Condition | mice | mice | STAM mice | STAM mice |
| Mean | 0 | 4.7 | 3.1 | 2.9 |
| stdev | 0 | 0.67 | 0.74 | 0.74 |

TABLE 16
NAFLD Activity: Steatosis Score

| | Steatosis | | | |
|---|---|---|---|---|
| | Normal | | Amino Acid | |
| | C57BL/6 | Vehicle-treated | Composition A-1 | OCA treated |
| | mice | STAM mice | treated STAM mice | STAM mice |
| Mean | 0 | 1 | 0.9 | 0.8 |
| stdev | 0 | 0.00 | 0.32 | 0.42 |

TABLE 17
NAFLD Activity: Inflammation Score

| | Inflammation | | | |
|---|---|---|---|---|
| | | Vehicle- | Amino Acid | OCA |
| | Normal | treated | Composition | treated |
| | C57BL/6 | STAM | A-1 treated | STAM |
| | mice | mice | STAM mice | mice |
| Mean | 0 | 2.1 | 1.8 | 1.8 |
| stdev | 0 | 0.32 | 0.63 | 0.79 |

TABLE 18
NAFLD Activity: Ballooning Score

| | Ballooning | | | |
|---|---|---|---|---|
| | | Vehicle- | Amino Acid | OCA |
| | Normal | treated | Composition | treated |
| | C57BL/6 | STAM | A-1 treated | STAM |
| | mice | mice | STAM mice | mice |
| Mean | 0 | 1.6 | 0.4 | 0.3 |
| stdev | 0 | 0.52 | 0.52 | 0.48 |

Fibrosis: Sirius Red Staining Results

Fibrosis was assessed by analysis of Sirius red positively stained cell area from stained liver sections from each animal. Images were quantified using the percent of positively stained area was used as a measure of fibrosis. Results of this analysis are shown in Table 19. Data are mean±standard deviation (stdev). Normal C57BL/6 mice fed standard chow had a mean positive area of 0.286+/−0.09. Vehicle treated STAM™ mice had a mean positive area of 1.1+/−0.26. Amino Acid Composition A-1 treated mice had a mean positive area of 0.828+/−0.33. OCA treated mice had a mean score of 0.776+/−0.25. Amino Acid Composition A-1 and OCA were statistically different from vehicle when compared using Dunnett's multiple comparisons test (Amino Acid Composition A-1 p=0.00494, OCA p<0.016). Raw data are shown in Table 19.

TABLE 19
Fibrosis (mean positively stained area, Sirius red)

| | | Vehicle- | Amino Acid | OCA |
|---|---|---|---|---|
| | Normal | treated | Composition | treated |
| | C57BL/6 | STAM | A-1 treated | STAM |
| Condition | mice | mice | STAM mice | mice |
| Mean | 0.286 | 1.1 | 0.828 | 0.776 |
| stdev | 0.09 | 0.26 | 0.33 | 0.25 |

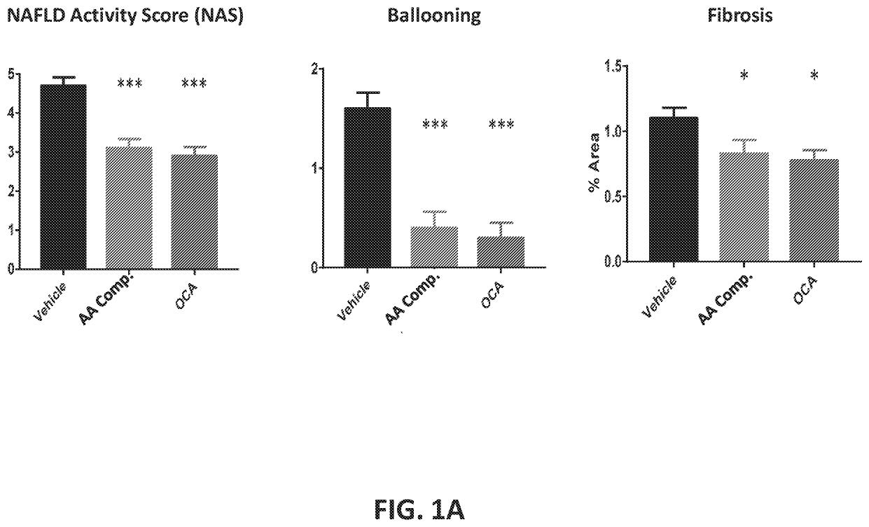

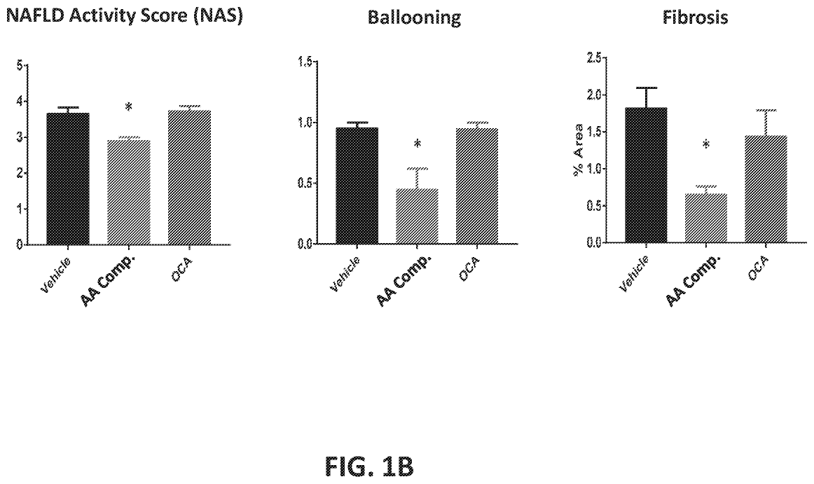

Similarly to the statistically significant improvement in the NAFLD activity score, ballooning, and fibrosis in the STAM mouse model after treatment with Amino Acid Composition A-1 (FIG. 1A), a statistically significant improvement in the NAFLD activity score, ballooning, and fibrosis was determined in the high-fat, high fructose and cholesterol diet (HFFC) mouse model after treatment with Amino Acid Composition A-1 (FIG. 1B).

α-Smooth Muscle Actin (α-SMA) Staining Results

Liver sections of all mice were stained for the marker α-smooth muscle actin (aSMA) to identify activated hepatic stellate cells. Images were quantified using the percent of positively stained area was used as a measure of stellate cell activation. Results are shown in Table 20. Data are mean±standard deviation (stdev); p values are compared to vehicle-treated STAM mice control; by one-tailed T test. Normal C57BL/6 mice fed standard chow had a mean positive area of 0.682+/−0.26. Vehicle treated STAM™ mice had a mean positive area of 2.128+/−0.50. Amino Acid Composition A-1 treated mice had a mean positive area of 1.657+/−0.84. OCA treated mice had a mean score of 1.562+/−0.31.

TABLE 20
Activated hepatic stellate cells (mean positively
stained area, α-smooth muscle actin)

| | | Vehicle- | Amino Acid | OCA |
|---|---|---|---|---|
| | Normal | treated | Composition | treated |
| | C57BL/6 | STAM | A-1 treated | STAM |
| Condition | mice | mice | STAM mice | mice |
| Mean | 0.682 | 2.128 | 1.657 | 1.562 |
| stdev | 0.26 | 0.50 | 0.84 | 0.31 |
| | | | p = 0.073 | p < 0.05 |

Treatment with Amino Acid Composition A-1 significantly reduced NASH severity to levels equivalent to Farnesoid X Receptor (FXR) inhibition by OCA (which is currently under clinical investigation by Intercept Pharmaceuticals, Inc. for treatment of NASH), as indicated by significant reduction in NAFLD Activity Score (NAS) (mean NAS: 3.1+/−0.74 for Amino Acid Composition A-1 vs. vehicle treated STAM™ mice mean score of 4.7+/−0.67, compared to OCA treated mice mean score of 2.9+/−0.74), and development of fibrosis as indicated by the downregulation of hepatic stellate cell activation (mean aSMA positively stained area: 1.657+/−0.84 for Amino Acid Composition A-1 vs. vehicle treated STAM™ mice mean area of 2.128+/−0.50, compared to OCA treated mice mean area of 1.562+/−0.31).

TABLE 21
NAFLD Activity Score: raw data

| | Vehicle- | Amino Acid | OCA |
|---|---|---|---|
| Normal | treated | Composition | treated |
| C57BL/6 | STAM | A-1 treated | STAM |
| mice | mice | STAM mice | mice |
| 0 | 6 | 3 | 4 |
| 0 | 5 | 4 | 2 |
| 0 | 5 | 4 | 2 |
| 0 | 4 | 3 | 4 |
| 0 | 5 | 2 | 3 |
| 0 | 5 | 2 | 3 |
| 0 | 4 | 3 | 2 |
| 0 | 4 | 3 | 3 |
| 0 | 4 | 3 | 3 |
| 0 | 5 | 4 | 3 |

TABLE 22
NAFLD Activity: Steatosis Score: raw data
Steatosis

| | Vehicle- | Amino Acid | OCA |
|---|---|---|---|
| Normal | treated | Composition | treated |
| C57BL/6 | STAM | A-1 treated | STAM |
| mice | mice | STAM mice | mice |
| 0 | 1 | 1 | 1 |
| 0 | 1 | 1 | 1 |
| 0 | 1 | 1 | 1 |
| 0 | 1 | 1 | 1 |
| 0 | 1 | 0 | 1 |
| 0 | 1 | 1 | 0 |
| 0 | 1 | 1 | 1 |
| 0 | 1 | 1 | 0 |
| 0 | 1 | 1 | 1 |
| 0 | 1 | 1 | 1 |

TABLE 23
NAFLD Activity: Inflammation Score: raw data
Inflammation

| | Vehicle- | Amino Acid | OCA |
|---|---|---|---|
| Normal | treated | Composition | treated |
| C57BL/6 | STAM | A-1 treated | STAM |
| mice | mice | STAM mice | mice |
| 0 | 3 | 1 | 2 |
| 0 | 2 | 2 | 1 |
| 0 | 2 | 2 | 1 |
| 0 | 2 | 2 | 2 |
| 0 | 2 | 1 | 2 |
| 0 | 2 | 1 | 3 |
| 0 | 2 | 2 | 1 |
| 0 | 2 | 2 | 3 |
| 0 | 2 | 2 | 2 |
| 0 | 2 | 3 | 1 |

TABLE 24
NAFLD Activity: Ballooning Score: raw data
Ballooning

| | Vehicle- | Amino Acid | OCA |
|---|---|---|---|
| Normal | treated | Composition | treated |
| C57BL/6 | STAM | A-1 treated | STAM |
| mice | mice | STAM mice | mice |
| 0 | 2 | 1 | 1 |
| 0 | 2 | 1 | 0 |
| 0 | 2 | 1 | 0 |
| 0 | 1 | 0 | 1 |
| 0 | 2 | 1 | 0 |
| 0 | 2 | 0 | 0 |
| 0 | 1 | 0 | 0 |
| 0 | 1 | 0 | 0 |
| 0 | 1 | 0 | 0 |
| 0 | 2 | 0 | 1 |

TABLE 25
Fibrosis (mean positively stained area, Sirius red): raw data

| | Vehicle- | Amino Acid | OCA |
|---|---|---|---|
| Normal | treated | Composition | treated |
| C57BL/6 | STAM | A-1 treated | STAM |
| mice | mice | STAM mice | mice |
| 0.26 | 0.79 | 1.07 | 0.36 |
| 0.35 | 1.43 | 0.58 | 0.56 |
| 0.19 | 1.44 | 0.48 | 1.1 |
| 0.31 | 1.36 | 0.58 | 1.19 |
| 0.19 | 1.04 | 1.07 | 0.89 |
| 0.36 | 0.75 | 0.34 | 0.91 |
| 0.24 | 1.07 | 0.86 | 0.66 |
| 0.37 | 1.13 | 1.43 | 0.72 |
| 0.18 | 0.83 | 0.96 | 0.68 |
| 0.41 | 1.16 | 0.91 | 0.69 |

TABLE 26
Activated hepatic stellate cells (mean positively
stained area, α-smooth muscle actin): raw data

| | Vehicle- | Amino Acid | OCA |
|---|---|---|---|
| Normal | treated | Composition | treated |
| C57BL/6 | STAM | A-1 treated | STAM |
| mice | mice | STAM mice | mice |
| 0.47 | 2.16 | 0.81 | 1.46 |
| 0.59 | 2.77 | 1.35 | 1.51 |
| 1.13 | 2.21 | 1.3 | 1.49 |
| 0.52 | 1.5 | 3.03 | 1.17 |
| 0.75 | 2.87 | 2.04 | 1.49 |
| 0.46 | 1.93 | 0.97 | 1.5 |
| 0.37 | 1.6 | 3.08 | 1.13 |
| 0.85 | 1.46 | 1.91 | 2.03 |
| 0.62 | 2.36 | 1.15 | 1.87 |
| 1.06 | 2.42 | 0.93 | 1.97 |

### Example 3. Reduction of Fibrogenic Gene Expression in Hepatic Stellate Cells Treated with an Amino Acid Composition

Hepatic stellate cells in a healthy liver are in the space of Disse, between the hepatocytes and liver sinusoidal endothelial cells. In response to liver injury hepatic stellate cells become activated, proliferative and contractile, increase production of aSMA, secretion of type I and III collagens and specific MMP and TIMP proteins. LX-2 cells were selected as a model of activated hepatic stellate cells and used to test whether specific amino acid compositions would reduce fibrogenic gene expression induced with TGFβ1.

LX-2 hepatic stellate cells (Millipore) were seeded on day 0 at 1.67E4 cells per well in collagen I coated 96-well microplates (ThermoFisher) in Dulbecco's Modified Eagle Medium (DMEM, Corning) supplemented with 2% heat inactivated fetal bovine serum (HI-FBS, HyClone) and 0.2% Primocin (InVivoGen) and incubated overnight at 37° C., 5% CO2. Cells were washed and media was replaced with amino acid free DMEM (US Biologicals) containing a defined custom amino acid concentration based on the mean physiological concentrations in blood based on values published in the Human Metabolome Database (1,2,3) and a dose curve of defined amino acid compositions LIVRQ+N-Acetylcysteine, LIVRQ, RQ+N-Acetylcysteine, N-acetylcysteine, LIV at 40× the concentration present in the basal HMDB (Human Metabolome Database (Wishart D S, Tzur D, Knox C, et al., HMDB: the Human Metabolome Database. Nucleic Acids Res. 2007 January; 35(Database issue):D521-6. 17202168)) derived amino acid concentrations or individually with leucine, isoleucine, valine, arginine, glutamine or cysteine at 50× the HMDB derived concentrations. Combinations containing N-acetylcysteine were dosed with 10 mM. Cells were pretreated for 6 hours at 37° C., 5% CO2. After pretreatment, TGFβ1 (R&D Systems) or vehicle was spiked into each well for a final concentration of 5 ng/mL and cells were incubated under this stimulus for a further 12 hours at 37° C., 5% CO2.

After 12 hour incubation, RNA extraction and quantitative PCR was conducted on lysates to determine collagen-1a1 expression normalized to β-actin housekeeping expression using the ΔΔCt method using TaqMan primer probes (Integrated DNA Technologies: Col1A1, Hs.PT.58.15517795; Actb, Hs.PT.39a.22214847; Acta2, Hs.PT.56a.24853961; Timp2, Hs.PT.58.14780594).

Table 27 shows the Col1a1, Acta2, and Timp2 gene expression in LX-2 cells treated with amino acid combinations compared to vehicle with or without TGFβ1 stimulus. LIVRQ+N-Acetylcysteine, LIVRQ, RQ+N-Acetylcysteine, and N-acetylcysteine reduced Col1a1 expression and Timp2 expression. LIVRQ+N-acetylcysteine shows the largest reduction of Col1a1, Acta2, and Timp2 gene expression. LIVRQ-N-acetylcysteine reduces Acta2 expression significantly greater than N-Acetylcysteine alone, RQ+N-acetylcysteine, and LIV. LIVRQ+N-acetylcysteine reduces Timp2 expression significantly greater than any of the other combinations (Table 27).

TABLE 27

| | | Col1a 1 | | | Acta2 | | | Timp2 | | |
|---|---|---|---|---|---|---|---|---|---|---|
| | Amino Acid | | Std. | Number of | | Std. | Number of | | Std. | Number of |
| TGFβ1 | Supplement | Mean | Deviation | values | Mean | Deviation | values | Mean | Deviation | values |
| Yes | Vehicle | 2.861 | 0.3151 | 4 | 0.801 | 0.1149 | 4 | 1.658 | 0.2791 | 4 |
| No | Vehicle | 1.042 | 0.3102 | 4 | 1.006 | 0.1190 | 4 | 1.022 | 0.2400 | 4 |
| Yes | LIVRQNAC | 1.267 | 0.4106 | 4 | 0.292 | 0.0969 | 4 | 0.535 | 0.0306 | 4 |
| Yes | LIVRQ | 1.787 | 0.2926 | 4 | 0.267 | 0.0637 | 4 | 0.975 | 0.2006 | 4 |
| Yes | RQNAC | 1.664 | 0.3320 | 4 | 0.487 | 0.1042 | 4 | 0.897 | 0.1932 | 4 |
| Yes | NAC | 1.659 | 0.4695 | 4 | 0.647 | 0.1097 | 4 | 1.076 | 0.0681 | 4 |
| Yes | LIV | 2.831 | 0.3404 | 3 | 0.793 | 0.0812 | 4 | 1.927 | 0.0944 | 4 |

Table 28 shows the Col1a1 expression of individual amino acids with or without TGFβ1 stimulus at 1× or 50× the HMDB derived amino acid concentration. Individually, only cysteine showed a significant decrease in Col1a1 expression at 50×.

TABLE 28

| | | Col1a1 | | |
|---|---|---|---|---|
| | Amino | | | Number |
| | Acid | | Std. | of |
| TGFβ1 | Supplement | Mean | Deviation | values |
| No | Vehicle | 1.015 | 0.1832 | 8 |
| Yes | 1X CYS | 2.491 | 0.1588 | 4 |
| Yes | 50X CYS | 1.695 | 0.3310 | 4 |
| Yes | 1X ILE | 2.020 | 0.1451 | 4 |
| Yes | 50X ILE | 2.028 | 0.3667 | 4 |
| Yes | 1X LEU | 1.901 | 0.3360 | 4 |
| Yes | 50X LEU | 2.372 | 0.4153 | 4 |
| Yes | 1X VAL | 2.093 | 0.2157 | 4 |
| Yes | 50X VAL | 2.203 | 0.5762 | 4 |
| No | Vehicle | 1.010 | 0.1510 | 8 |
| Yes | 1X ARG | 1.620 | 0.6691 | 4 |
| Yes | 50X ARG | 1.970 | 0.7740 | 4 |
| No | Vehicle | 1.012 | 0.1681 | 8 |
| Yes | 1X GLN | 2.340 | 0.7069 | 4 |
| Yes | 50X GLN | 2.194 | 0.3359 | 4 |

### Example 4. Treatment with an Amino Acid Composition Ameliorates NASH Progression in Two Rodent Models by Impacting Lipid Metabolism, and Fibrosis

The amino acid composition is formulated to simultaneously target multiple mechanisms of disease pathology to safely and effectively treat NASH (Table 29). As described herein, the efficacy of the amino acid composition was studied in two established mouse models of NASH to determine the effect of the amino acid composition on signs and symptoms associated with NASH and related disorders.

TABLE 29
Exemplary amino acid components of the amino acid composition.

| Amino acid | wt. ratio | wt. % | g/packet | g dose #1 | | g dose #2 | |
|---|---|---|---|---|---|---|---|
| Leucine | 1 | 16.78 | 1.00 g | 2 | g | 4 | g |
| Isoleucine | 0.5 | 8.39 | 0.50 g | 1 | g | 2 | g |
| Valine | 0.5 | 8.39 | 0.50 g | 1 | g | 2 | g |
| Arginine HCl | 1.81 | 30.37 | 1.81 g | 3.62 | g | 7.24 | g |
| Glutamine | 2 | 33.56 | 2.00 g | 4 | g | 8 | g |
| N-acetylcysteine | 0.15 | 2.52 | 0.15 g | 0.3 | g | 0.6 | g |
| Total amino acids | | | 5.96 g | ~12 | g | ~24 | g |

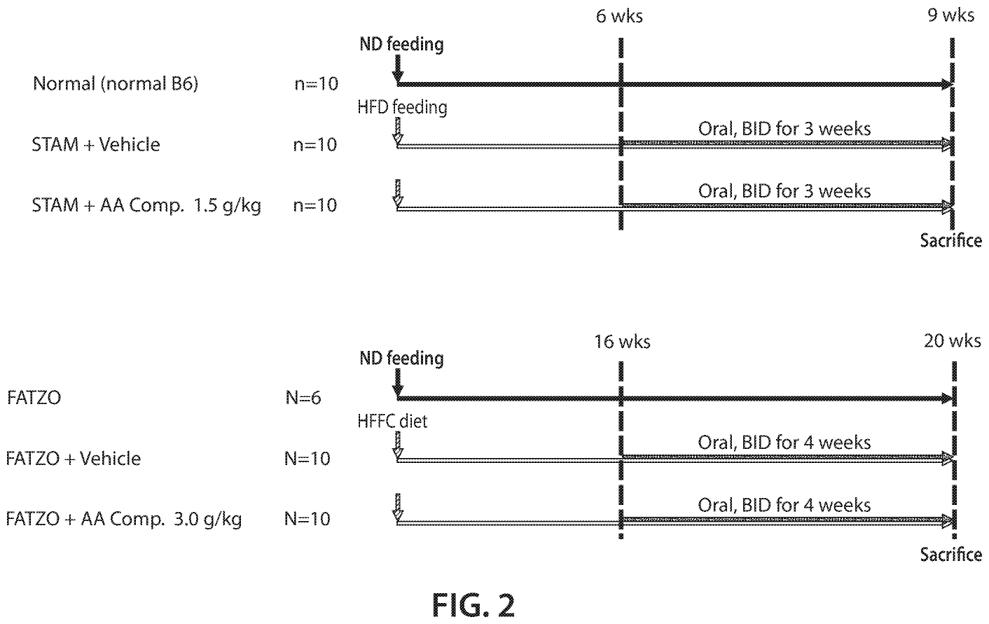

The STAM™ mouse is a model for non-alcoholic steatohepatitis (NASH) and hepatocellular carcinoma (HCC), developed by SMC Laboratories, Inc. Evidence of fatty liver is present by 5 weeks of age, followed by NASH by 7 weeks of age, and fibrosis by 9 weeks of age. Male STAM mice were generated in C57BL/6 mice, which received a low dose streptozotocin 2 days after birth and were fed a high fat diet (57% kcal fat, HFD32, CLEA Japan, Inc.) starting at 4 weeks old (Saito K. et al., 2015 Sci Rep 5: 12466; hereby incorporated by reference in its entirety). The amino acid composition was administered to STAM mice at a dose of 1.6 m/kg twice daily for 3 weeks starting at 6 weeks of age. One group of vehicle treated STAM mice was included as a control. Unfasted mice were euthanized at 9 weeks old. Plasma and liver samples were harvested for further analysis (FIG. 2).

The FATZO™ mouse is an inbred, polygenic model of obesity, metabolic syndrome, and NASH, developed by Crown Bioscience, Inc (Peterson R G. Et al., 2017 PLoS One; hereby incorporated by reference in its entirety). Male FATZO mice were fed a high fat, fructose, and cholesterol (HFFC) diet (40% kcal fat, D12079B, Research Diets, Inc. and 5% fructose in drinking water) starting at 6 weeks old to induce NAFLD and NASH. Evidence of fatty liver is present by 4 weeks post induction, followed by NASH by 16 weeks post induction and fibrosis by 20 weeks of induction. The designed amino acid composition was administered at a dose of 3.0 g/kg twice daily for 4 weeks starting at 16 weeks post induction (FIG. 2). One group of vehicle treated FATZO mice was included as control. Unfasted mice were euthanized at 20 weeks post-induction. Plasma and liver samples were harvested for further analysis.

The Aperio ScanScope CS whole slide digital imaging system (Vista, Calif.) was used for imaging in H&E, Picric Sirius Red, SMA, F4/80. Images were captured from whole slides.

The livers were evaluated by veterinary pathologists blind to sample ID using the NASH Clinical Research Network (CRN) liver histological scoring system (Kleiner D E, et al., 2015, hereby incorporated by reference in its entirety). The NASH CRN Scoring System assesses progression of steatosis, lobular inflammation, hepatocyte ballooning, degeneration, and fibrosis. One cross section of liver for each case was analyzed with the NASH score system. Steatosis, lobular inflammation, and fibrosis progression was assessed on a 0-3 scale. Ballooning degeneration was assessed on a 0-2 scale.

The Positive Pixel Count algorithm of the Aperio Automatic Image Quantitation was used to quantify the percentage of a specific stain present in a scanned slide image. A range of color (range of hues and saturation) and three intensity ranges (weak, positive, and strong) were masked and evaluated. The algorithm counted the number and intensity-sum in each intensity range, along with three additional quantities: average intensity, ratio of strong/total number, and average intensity of weak positive pixels.

A specific positive pixel algorithm was used for imaging the Sirius Red and Oil Red 0 liver sections. The positive pixel algorithm was modified to distinguish between the orange and blue colors. Alterations from the normal “hue value” (0.1 to 0.96) and “color saturation” (0.04 to 0.29), were made for the Sirius Red evaluation. Vasculature and artifacts were excluded from analysis.

Liver total lipid-extracts were obtained by Folch's method (Folch J. et al., J. Biol. Chem. 1957; 226: 497; hereby incorporated by reference in its entirety). Liver samples were homogenized in chloroform-methanol (2:1, v/v) and incubated overnight at room temperature. After washing with chloroform-methanol-water (8:4:3, v/v/v), the extracts were evaporated to dryness, and dissolved in isopropanol. Liver triglyceride and cholesterol contents were measured by the Triglyceride E-test and Cholesterol E-test, respectively.

Liver RNA samples were converted into cDNA libraries using the Illumina TruSeq Stranded mRNA sample preparation kit (Illumina #RS-122-2103). Transcriptome were analyzed at Q2 Solutions (Morrisville, N.C.). RNA Seq data were normalized and analyzed using Ingenuity Pathway Analysis (QIAGEN Bioinformatics). Mouse liver gene expression at the pathway level was focused on because it is translatable to human NAFLD (Teufel A, et al., Gastroenterology, 2016, hereby incorporated by reference in its entirety).

Metabolic profiling based on both capillary electrophoresis time-of-flight mass spectrometry (CE-TOFMS) and LC-TOFMS platforms was performed at Human Metabolome Technologies (Yamagata, Japan). Metabolites in the samples were identified by comparing the migration time and m/z ratio with authentic standards and quantified by comparing their peak areas with those of authentic standards.

The level of IL-1b protein in liver was quantified using the multiplex ELISA Assay (Meso Scale Discovery, Rockville, Md.).

The Amino Acid Composition Improves Ballooning and Fibrosis in Both STAM and FATZO Mice

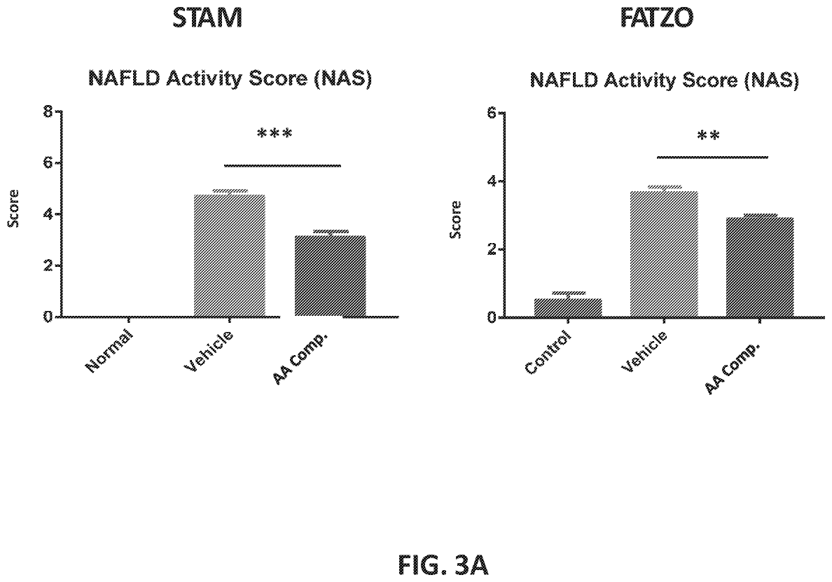

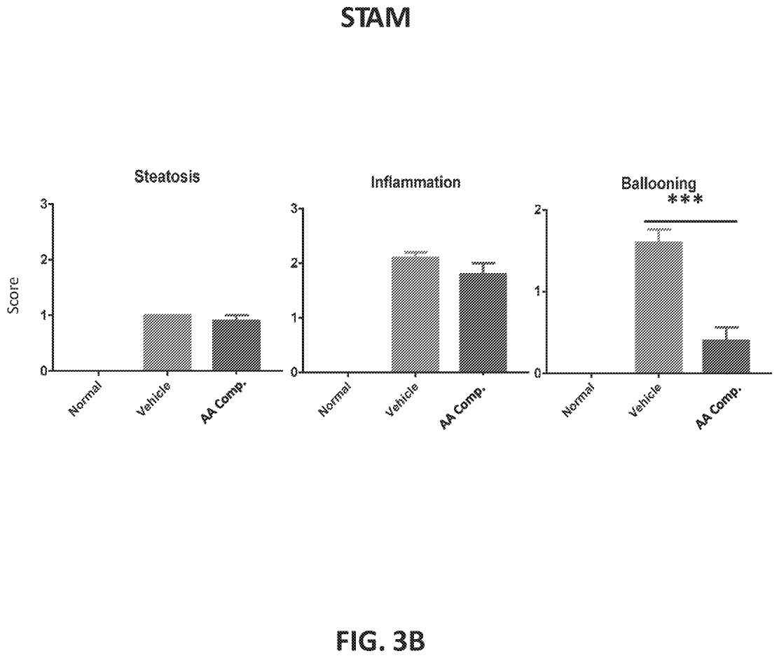

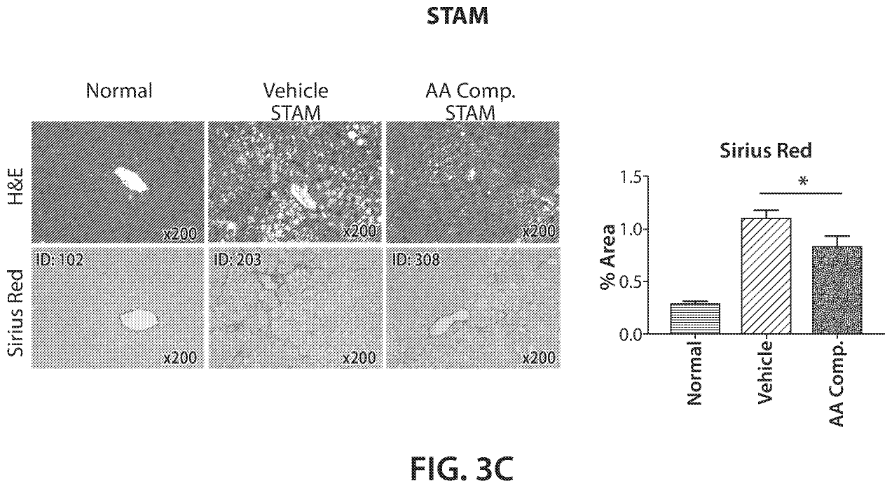

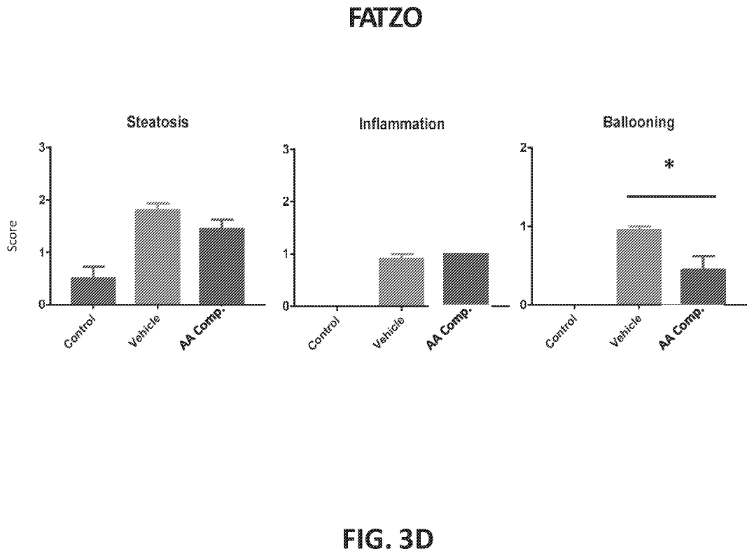

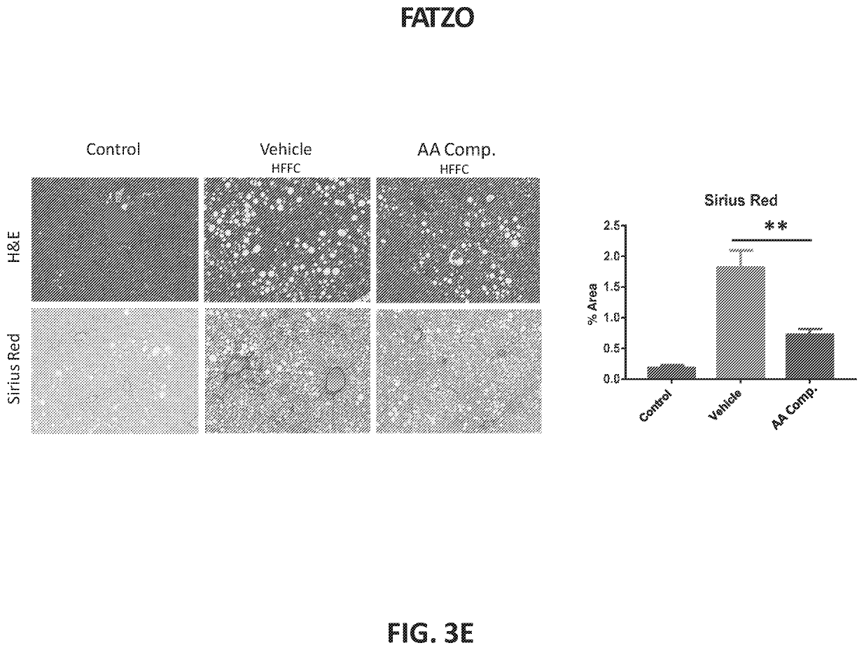

Treatment with the amino acid composition significantly reduced NAFLD activity scores (NAS) in both STAM and FATZO mice (FIG. 3A). Treatment with the amino acid composition also significantly decreased hepatocyte ballooning in STAM mice (FIG. 3B). Scores of steatosis and inflammation were not changed according to histological measures by treatment of STAM mice with the amino acid composition. The Sirius Red-positive, fibrosis area was significantly lowered by treating the STAM mice with the amino acid composition, while the Oil Red O area was not changed by treating the STAM mice with the amino acid composition (FIG. 3C). Liver triglyceride and cholesterol levels were not changed.

Treatment with the amino acid composition also significantly decreased hepatocyte ballooning in FATZO mice (FIG. 3D). Scores of steatosis and inflammation as well as liver triglyceride and cholesterol levels were not changed in the FATZO mice treated with the amino acid composition treatment. The Sirius Red-positive, fibrosis area was significantly lowered by treatment of the FATZO mice with the amino acid composition, while the Oil Red 0 area was not changed by treatment of the FATZO mice with the amino acid composition treatment (FIG. 3E).

The Amino Acid Composition Prevents Fibrogenesis Pathways

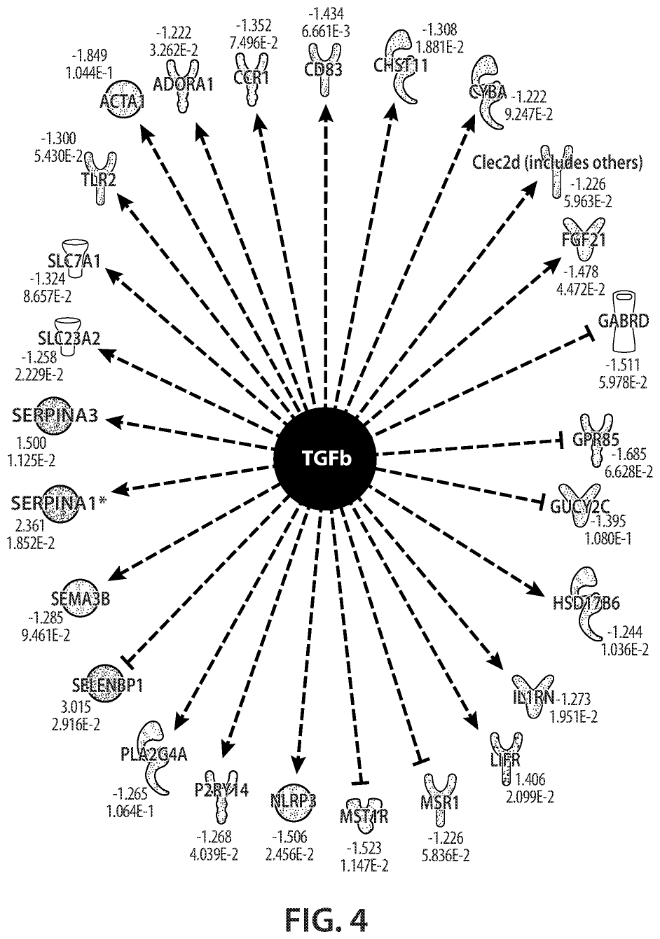

Fibrosis is at the nexus of several biologic processes, such as metabolic dysregulation, inflammation, and cell death. Lipid accumulation in hepatocytes and chronic inflammation induce fibrogenic activation of hepatic stellate cells (Wobser H, et al., Cell Res. 2009, which is hereby incorporated by reference in its entirety). The liver gene expression pattern resulting from treatment with the amino acid composition was consistent with the suppression of the fibrogenic TGF-b signaling pathway (FIG. 4).

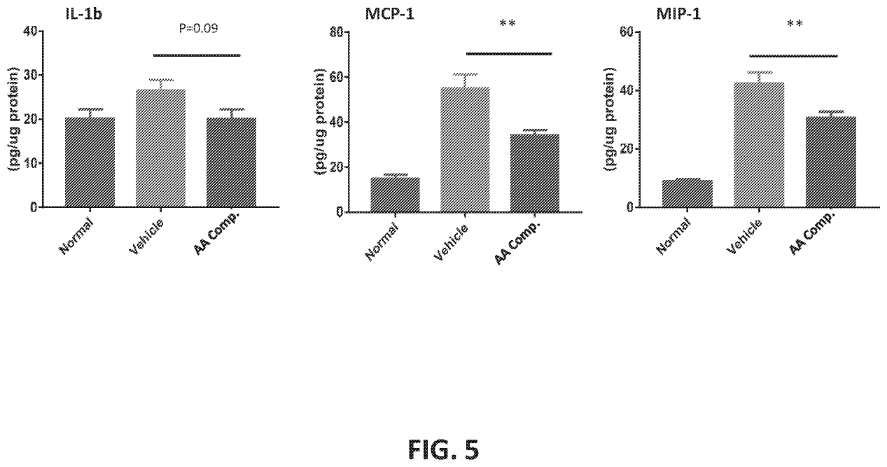

Increasing evidence implicates that CCR2/CCR5 and their ligands, including MCP-1/MIP-1, promote macrophage recruitment and hepatic stellate cell activation which contribute to fibrosis following liver tissue damage (Lefebvre E, et al., PLoS One 2016, which is hereby incorporated by reference in its entirety). The amino acid composition displayed a potent antifibrotic activity in the STAM model of NASH via reducing hepatic TGF-b signaling and MCP-1 and MIP-1 proteins (FIG. 5).

The amino acid composition demonstrated consistent disease modifying activity in both STAM and FATZO mouse models of NASH including improvement in NAS and amelioration of ballooning and fibrosis. The activity of the amino acid composition appears to be driven, at least in part, via increase in fatty acid oxidation, reduction in levels of transcription pathways associated with fibrosis.

### Example 5. TGFβ1 Fibrogenic Gene Expression of Hepatic Stellate Cell

Primary human hepatic stellate cells were obtained from Samsara Sciences. Cells were grown in Complete HSC Medium to ˜80% confluence in T75 or T150 flasks below passage 10 were seeded into sterile, collagen I coated, 96-well optical plastic microplates (ThermoScientific, 152036) and incubated overnight at 37° C., 5% CO2 in a humidified incubator in DMEM with 2% Fetal Bovine Serum and 1% Antibiotic-Antimycotic. After the overnight incubation, plates are washed and pretreated with medium±single amino acid dropout, 1×HMDB DMEM±supplemental amino acid dose for 10.5 hours. After 10.5 hour pretreatment, the same pretreatment medium supplemented with 3 ng/mL TGFβ1, was applied and incubated for 24 hours at 37° C., 5% CO2. After 24 hour stimulus, supernatant was removed, RNA was extracted and gene expression was evaluated using the ΔΔCq method within each single amino acid dropout and supplementation by normalizing to its own 1×HMDB concentration.

Human Procollagen 1α1 was measured from the supernatant by ELISA (Human ProCollagen I alpha 1 DuoSet ELISA, R&D Systems) at 1/100 dilution in 1× Reagent Diluent (Reagent Ancillary Kit 2, R&D Systems).

### Col1a1 Gene Expression

Tables 30, 31, 31-1, 31-2, 31-3, and 31-4 show the mean fold change in Col1a1 gene expression in primary human hepatic stellate cells from three different healthy donors. LIVRQNAC and LIVRQNAC+S showed significantly decreased Col1a1 gene expression in two of three donors. LIVRQNAC+G and RQNAC showed significantly decreased Col1a1 expression in all three donors. LIVRQ showed a significant change in Col1a1 gene expression in only one donor. LIV alone did not significantly change Col1a1 gene expression.

Each of leucine, isoleucine, valine, and arginine did not significantly change Col1a1 gene expression in any donor when the amino acid was administered alone. Glutamine decreased Col1a1 gene expression in two of three donors. N-acetyl cysteine significantly reduced Col1a1 gene expression in all three donors.

Fold change of Col1a1 gene expression after administration of an amino acid composition,
normalized to Gapdh expression in a first donor

| | | Col1a1 Fold Expression Relative to Control | | | | |
|---|---|---|---|---|---|---|
| | | | Std. | Number | | |
| Amino Acid Supplement | Conc. (X) | Mean | Deviation | of values | P-value* | Significance |
| LIVRQNAC | 40 | 0.91 | 0.08 | 4 | ns | 0.401 |
| LIVRQNAC | 30 | 0.87 | 0.10 | 4 | ns | 0.1073 |
| LIVRQNAC | 20 | 0.88 | 0.04 | 4 | ns | 0.1483 |
| LIVRQNAC | 10 | 0.90 | 0.08 | 4 | ns | 0.3035 |
| LIVRQNAC | 1 | 1.00 | 0.10 | 4 | | |
| LIVRQNAC + G | 40 | 0.73 | 0.15 | 4 | ** | 0.0053 |
| LIVRQNAC + G | 30 | 0.79 | 0.08 | 4 | * | 0.0252 |
| LIVRQNAC + G | 20 | 0.84 | 0.08 | 4 | ns | 0.1181 |
| LIVRQNAC + G | 10 | 0.79 | 0.11 | 4 | * | 0.0286 |
| LIVRQNAC + G | 1 | 1.00 | 0.03 | 4 | | |
| LIVRQNAC + S | 40 | 0.79 | 0.05 | 4 | * | 0.0325 |
| LIVRQNAC + S | 30 | 0.86 | 0.13 | 4 | ns | 0.1848 |
| LIVRQNAC + S | 20 | 0.96 | 0.10 | 4 | ns | 0.9287 |
| LIVRQNAC + S | 10 | 0.85 | 0.12 | 4 | ns | 0.1566 |
| LIVRQNAC + S | 1 | 1.00 | 0.10 | 4 | | |
| LIV | 40 | 0.93 | 0.03 | 4 | ns | 0.5561 |
| LIV | 30 | 1.04 | 0.07 | 4 | ns | 0.8872 |
| LIV | 20 | 1.04 | 0.09 | 4 | ns | 0.9069 |
| LIV | 10 | 1.05 | 0.10 | 4 | ns | 0.8156 |
| LIV | 1 | 1.00 | 0.07 | 4 | | |
| LIVRQ | 40 | 0.75 | 0.03 | 4 | *** | 0.001 |
| LIVRQ | 30 | 0.73 | 0.05 | 4 | *** | 0.0004 |
| LIVRQ | 20 | 0.80 | 0.03 | 4 | ** | 0.0054 |
| LIVRQ | 10 | 0.84 | 0.08 | 4 | * | 0.0208 |
| LIVRQ | 1 | 1.01 | 0.13 | 4 | | |
| RQNAC | 40 | 0.51 | 0.07 | 4 | **** | 0.0001 |
| RQNAC | 30 | 0.49 | 0.02 | 4 | **** | 0.0001 |
| RQNAC | 20 | 0.59 | 0.04 | 4 | **** | 0.0001 |
| RQNAC | 10 | 0.68 | 0.07 | 4 | **** | 0.0001 |
| RQNAC | 1 | 1.00 | 0.11 | 4 | | |
| N-Acetyl Cysteine | 40 | 0.76 | 0.06 | 4 | ** | 0.0011 |
| N-Acetyl Cysteine | 20 | 1.02 | 0.08 | 4 | ns | 0.9921 |
| N-Acetyl Cysteine | 10 | 1.07 | 0.08 | 4 | ns | 0.5517 |
| N-Acetyl Cysteine | 5 | 1.00 | 0.08 | 4 | ns | 0.9999 |
| N-Acetyl Cysteine | 0 | 1.00 | 0.06 | 4 | | |

TABLE 31
Fold change of Col1a1 gene expression after administration of a single amino acid composition,
normalized to Gapdh expression in the first donor

| | | Col1a1 Fold Expression Relative to Control | | | | |
|---|---|---|---|---|---|---|
| | Conc. | | Std. | Number | | |
| Amino Acid Supplement | (μM) | Mean | Deviation | of values | P-value* | Significance |
| Valine | 23420 | 1.00 | 0.05 | 4 | ns | 0.9996 |
| Valine | 11710 | 1.09 | 0.17 | 4 | ns | 0.5528 |
| Valine | 4684 | 1.05 | 0.11 | 4 | ns | 0.8851 |
| Valine | 234 | 1.00 | 0.08 | 4 | | |
| Arginine | 5440 | 1.12 | 0.18 | 4 | ns | 0.2151 |
| Arginine | 2720 | 1.03 | 0.03 | 4 | ns | 0.9625 |
| Arginine | 1088 | 0.99 | 0.06 | 4 | ns | 0.9989 |
| Arginine | 109 | 1.00 | 0.03 | 4 | | |
| Glutamine | 22484 | 0.53 | 0.01 | 4 | **** | 0.0001 |
| Glutamine | 11242 | 0.62 | 0.05 | 4 | **** | 0.0001 |
| Glutamine | 3747 | 0.70 | 0.03 | 3 | **** | 0.0001 |
| Glutamine | 749 | 1.00 | 0.07 | 4 | ns | 0.9999 |
| Glutamine | 562 | 1.00 | 0.07 | 3 | | |
| Isoleucine | 6639 | 1.11 | 0.07 | 4 | ns | 0.7553 |
| Isoleucine | 3320 | 1.10 | 0.14 | 4 | ns | 0.7944 |
| Isoleucine | 1328 | 1.05 | 0.22 | 4 | ns | 0.9831 |
| Isoleucine | 66 | 1.01 | 0.21 | 4 | | |
| Leucine | 15270 | 0.99 | 0.10 | 4 | ns | 0.994 |
| Leucine | 7635 | 1.12 | 0.16 | 4 | ns | 0.5049 |
| Leucine | 3054 | 1.11 | 0.15 | 4 | ns | 0.5499 |
| Leucine | 153 | 1.00 | 0.11 | 4 | | |
| N-Acetyl Cysteine | 10000 | 0.76 | 0.06 | 4 | ** | 0.0011 |
| N-Acetyl Cysteine | 5000 | 1.02 | 0.08 | 4 | ns | 0.9921 |
| N-Acetyl Cysteine | 2500 | 1.07 | 0.08 | 4 | ns | 0.5517 |
| N-Acetyl Cysteine | 1000 | 1.00 | 0.08 | 4 | ns | 0.9999 |
| N-Acetyl Cysteine | 0 | 1.00 | 0.06 | 4 | | |

TABLE 31-1
Fold change of Col1a1 gene expression after administration of an amino acid composition,
normalized to Gapdh expression in second donor.

| | | Col1a1 Fold Expression Relative to Control | | | | |
|---|---|---|---|---|---|---|
| | | | Std. | Number | | |
| Amino Acid Supplement | Conc. (X) | Mean | Deviation | of values | P-value* | Significance |
| LIVRQNAC | 40 | 0.72 | 0.05 | 4 | **** | 0.0001 |
| LIVRQNAC | 30 | 0.72 | 0.02 | 4 | **** | 0.0001 |
| LIVRQNAC | 20 | 0.70 | 0.03 | 4 | **** | 0.0001 |
| LIVRQNAC | 10 | 0.71 | 0.08 | 4 | **** | 0.0001 |
| LIVRQNAC | 1 | 1.00 | 0.02 | 4 | | |
| LIVRQNAC + G | 40 | 0.60 | 0.09 | 4 | **** | 0.0001 |
| LIVRQNAC + G | 30 | 0.68 | 0.07 | 4 | *** | 0.0001 |
| LIVRQNAC + G | 20 | 0.71 | 0.09 | 4 | *** | 0.0003 |
| LIVRQNAC + G | 10 | 0.69 | 0.06 | 4 | *** | 0.0002 |
| LIVRQNAC + G | 1 | 1.00 | 0.07 | 4 | | |
| LIVRQNAC + S | 40 | 0.66 | 0.02 | 4 | **** | 0.0001 |
| LIVRQNAC + S | 30 | 0.69 | 0.06 | 4 | **** | 0.0001 |
| LIVRQNAC + S | 20 | 0.76 | 0.05 | 4 | *** | 0.0002 |
| LIVRQNAC + S | 10 | 0.77 | 0.04 | 4 | *** | 0.0003 |
| LIVRQNAC + S | 1 | 1.00 | 0.11 | 4 | | |
| LIV | 40 | 1.20 | 0.21 | 4 | ns | 0.1032 |
| LIV | 30 | 1.10 | 0.09 | 4 | ns | 0.6074 |
| LIV | 20 | 1.10 | 0.04 | 4 | ns | 0.6031 |
| LIV | 10 | 1.02 | 0.08 | 4 | ns | 0.9981 |
| LIV | 1 | 1.00 | 0.11 | 4 | | |
| LIVRQ | 40 | 1.23 | 0.13 | 4 | ns | 0.1945 |
| LIVRQ | 30 | 1.12 | 0.13 | 4 | ns | 0.7176 |
| LIVRQ | 20 | 1.08 | 0.24 | 4 | ns | 0.8874 |
| LIVRQ | 10 | 1.14 | 0.16 | 4 | ns | 0.5632 |
| LIVRQ | 1 | 1.00 | 0.11 | 4 | | |
| RQNAC | 40 | 0.54 | 0.03 | 4 | **** | 0.0001 |
| RQNAC | 30 | 0.55 | 0.06 | 4 | **** | 0.0001 |
| RQNAC | 20 | 0.58 | 0.04 | 4 | **** | 0.0001 |
| RQNAC | 10 | 0.73 | 0.04 | 4 | *** | 0.0007 |
| RQNAC | 1 | 1.01 | 0.16 | 4 | | |
| N-Acetyl Cysteine | 40 | 0.57 | 0.06 | 4 | **** | 0.0001 |
| N-Acetyl Cysteine | 20 | 0.69 | 0.06 | 4 | **** | 0.0001 |
| N-Acetyl Cysteine | 10 | 0.69 | 0.09 | 4 | *** | 0.0001 |
| N-Acetyl Cysteine | 5 | 0.69 | 0.05 | 4 | *** | 0.0001 |
| N-Acetyl Cysteine | 0 | 1.00 | 0.10 | 4 | | |

TABLE 31-2
Fold change of Col1a1 gene expression after administration of a single amino acid composition,
normalized to Gapdh expression in second donor.

| | | Col1a1 Fold Expression Relative to Control | | | | |
|---|---|---|---|---|---|---|
| | Conc. | | Std. | Number | | |
| Amino Acid Supplement | (μM) | Mean | Deviation | of values | P-value* | Significance |
| Valine | 23420 | 1.05 | 0.03 | 4 | ns | 0.9194 |
| Valine | 11710 | 0.98 | 0.11 | 4 | ns | 0.9827 |
| Valine | 4684 | 1.05 | 0.18 | 4 | ns | 0.8893 |
| Valine | 234 | 1.00 | 0.11 | 4 | | |
| Arginine | 5440 | 1.15 | 0.10 | 4 | ns | 0.2773 |
| Arginine | 2720 | 1.15 | 0.14 | 4 | ns | 0.2759 |
| Arginine | 1088 | 0.99 | 0.15 | 4 | ns | 0.9938 |
| Arginine | 109 | 1.00 | 0.12 | 4 | | |
| Glutamine | 22484 | 0.86 | 0.07 | 4 | ns | 0.1411 |
| Glutamine | 11242 | 0.91 | 0.09 | 4 | ns | 0.4365 |
| Glutamine | 3747 | 1.04 | 0.14 | 4 | ns | 0.9811 |
| Glutamine | 749 | 1.02 | 0.13 | 4 | ns | 0.9988 |
| Glutamine | 562 | 1.01 | 0.12 | 8 | | |
| Isoleucine | 6639 | 1.03 | 0.07 | 4 | ns | 0.8931 |
| Isoleucine | 3320 | 0.99 | 0.08 | 4 | ns | 0.9841 |
| Isoleucine | 1328 | 0.97 | 0.10 | 4 | ns | 0.9157 |
| Isoleucine | 66 | 1.00 | 0.02 | 4 | | |
| Leucine | 15270 | 1.13 | 0.14 | 4 | ns | 0.0811 |
| Leucine | 7635 | 1.05 | 0.05 | 4 | ns | 0.7277 |
| Leucine | 3054 | 1.06 | 0.03 | 4 | ns | 0.5342 |
| Leucine | 153 | 1.00 | 0.03 | 4 | | |
| N-Acetyl Cysteine | 10000 | 0.57 | 0.06 | 4 | **** | 0.0001 |
| N-Acetyl Cysteine | 5000 | 0.69 | 0.06 | 4 | **** | 0.0001 |
| N-Acetyl Cysteine | 2500 | 0.69 | 0.09 | 4 | *** | 0.0001 |
| N-Acetyl Cysteine | 1000 | 0.69 | 0.05 | 4 | *** | 0.0001 |
| N-Acetyl Cysteine | 0 | 1.00 | 0.10 | 4 | | |

TABLE 31-3
Fold change of Col1a1 gene expression after administration of an amino acid composition,
normalized to Gapdh expression in third donor.

| | | Col1a1 Fold Expression Relative to Control | | | | |
|---|---|---|---|---|---|---|
| | | | Std. | Number | | |
| Amino Acid Supplement | Conc. (X) | Mean | Deviation | of values | P-value* | Significance |
| LIVRQNAC | 40 | 0.81 | 0.09 | 4 | ** | 0.008 |
| LIVRQNAC | 30 | 0.70 | 0.06 | 4 | *** | 0.0001 |
| LIVRQNAC | 20 | 0.79 | 0.08 | 4 | ** | 0.0035 |
| LIVRQNAC | 10 | 0.79 | 0.07 | 4 | ** | 0.0039 |
| LIVRQNAC | 1 | 1.00 | 0.06 | 4 | | |
| LIVRQNAC + G | 40 | 0.63 | 0.10 | 4 | *** | 0.0002 |
| LIVRQNAC + G | 30 | 0.64 | 0.02 | 4 | *** | 0.0003 |
| LIVRQNAC + G | 20 | 0.75 | 0.14 | 4 | ** | 0.005 |
| LIVRQNAC + G | 10 | 0.71 | 0.11 | 4 | ** | 0.0017 |
| LIVRQNAC + G | 1 | 1.00 | 0.03 | 4 | | |
| LIVRQNAC + S | 40 | 0.79 | 0.11 | 4 | * | 0.0316 |
| LIVRQNAC + S | 30 | 0.79 | 0.04 | 4 | * | 0.0309 |
| LIVRQNAC + S | 20 | 0.77 | 0.09 | 4 | * | 0.0208 |
| LIVRQNAC + S | 10 | 0.85 | 0.09 | 4 | ns | 0.1434 |
| LIVRQNAC + S | 1 | 1.01 | 0.16 | 4 | | |
| LIV | 40 | 1.00 | 0.16 | 4 | ns | 0.9999 |
| LIV | 30 | 0.94 | 0.16 | 4 | ns | 0.8685 |
| LIV | 20 | 1.08 | 0.08 | 4 | ns | 0.6767 |
| LIV | 10 | 0.93 | 0.04 | 4 | ns | 0.7713 |
| LIV | 1 | 1.00 | 0.05 | 4 | | |
| LIVRQ | 40 | 1.00 | 0.05 | 4 | ns | 0.9999 |
| LIVRQ | 30 | 1.07 | 0.13 | 4 | ns | 0.8753 |
| LIVRQ | 20 | 1.10 | 0.13 | 4 | ns | 0.6983 |
| LIVRQ | 10 | 1.05 | 0.21 | 4 | ns | 0.9641 |
| LIVRQ | 1 | 1.00 | 0.07 | 4 | | |
| RQNAC | 40 | 0.64 | 0.05 | 4 | *** | 0.0003 |
| RQNAC | 30 | 0.70 | 0.13 | 4 | ** | 0.0018 |
| RQNAC | 20 | 0.66 | 0.05 | 4 | *** | 0.0005 |
| RQNAC | 10 | 0.87 | 0.15 | 4 | ns | 0.2175 |
| RQNAC | 1 | 1.00 | 0.04 | 4 | | |
| N-Acetyl Cysteine | 40 | 0.62 | 0.01 | 4 | *** | 0.0005 |
| N-Acetyl Cysteine | 20 | 0.73 | 0.10 | 4 | ** | 0.0083 |
| N-Acetyl Cysteine | 10 | 0.82 | 0.09 | 4 | ns | 0.0909 |
| N-Acetyl Cysteine | 5 | 0.91 | 0.12 | 4 | ns | 0.4954 |
| N-Acetyl Cysteine | 0 | 1.01 | 0.16 | 4 | | |

TABLE 31-4
Fold change of Col1a1 gene expression after administration of a single amino acid composition,
normalized to Gapdh expression in second donor.

| | | Col1a1 Fold Expression Relative to Control | | | | |
|---|---|---|---|---|---|---|
| Amino Acid | Conc. | | Std. | Number | | |
| Supplement | (μM) | Mean | Deviation | of values | P-value* | Significance |
| Valine | 23420 | 1.13 | 0.12 | 4 | ns | 0.7199 |
| Valine | 11710 | 1.27 | 0.31 | 4 | ns | 0.1735 |
| Valine | 4684 | 1.22 | 0.16 | 4 | ns | 0.3247 |
| Valine | 234 | 1.01 | 0.13 | 4 | | |
| Arginine | 5440 | 1.02 | 0.09 | 4 | ns | 0.9702 |
| Arginine | 2720 | 0.99 | 0.09 | 4 | ns | 0.9973 |
| Arginine | 1088 | 0.95 | 0.02 | 4 | ns | 0.5384 |
| Arginine | 109 | 1.00 | 0.05 | 4 | | |
| Glutamine | 22484 | 0.81 | 0.11 | 4 | * | 0.0113 |
| Glutamine | 11242 | 0.81 | 0.11 | 4 | ** | 0.0087 |
| Glutamine | 3747 | 1.00 | 0.03 | 4 | ns | 0.9999 |
| Glutamine | 749 | 0.96 | 0.07 | 4 | ns | 0.8697 |
| Glutamine | 562 | 1.00 | 0.10 | 8 | | |
| Isoleucine | 6639 | 1.03 | 0.04 | 4 | ns | 0.9974 |
| Isoleucine | 3320 | 0.94 | 0.13 | 4 | ns | 0.8329 |
| Isoleucine | 1328 | 0.94 | 0.17 | 4 | ns | 0.7947 |
| Isoleucine | 66 | 1.02 | 0.20 | 4 | | |
| Leucine | 15270 | 1.07 | 0.12 | 4 | ns | 0.9535 |
| Leucine | 7635 | 1.00 | 0.16 | 4 | ns | 0.998 |
| Leucine | 3054 | 1.08 | 0.23 | 4 | ns | 0.9185 |

### Procollagen Iα1 Secretion

Tables 32, 33, 33-1, 33-2, 33-3, and 33-4 show the fold change in procollagen Iα1 in primary human hepatic stellate cells from three different healthy donors normalized to their respective baseline amino acid conditions. Statistical significance calculated by one-way

ANOVA with Dunnett's multiple comparison test within each treatment group. The combination LIV significantly increased procollagen Iα1 secretion in all three donors. The addition of arginine (R) and glutamine (Q) to a combination of LIV counteracted the profibrogenic effect of LIV alone. LIVRQNAC, LIVRQNAC+G, LIVRQNAC+S and RQNAC significantly decreased procollagen Iα1 secretion in all three donors. Individually, N-acetyl cysteine was shown to significantly decrease procollagen Iα1 secretion in two of the three donors. Valine significantly increased procollagen Iα1 secretion in only one of two donors, while isoleucine and arginine significantly increased procollagen Iα1 secretion in two of three donors. In other words, glutamine administered individually did not have a significant impact on procollagen Iα1 secretion. As such, the reduction of the profibrogenic effect of LIV with arginine and glutamine relative to that of LIV alone would not have been expected based on the effect of individual amino acid treatments.

TABLE 32
Fold change of procollagen 1α1 secretion after administration of an amino
acid composition in a first donor

| | | Procollagen Ia1 Secretion (Fold Change of 1X) | | | | |
|---|---|---|---|---|---|---|
| | | | Std. | Number | | |
| Amino Acid Supplement | Conc. (X) | Mean | Deviation | of values | P-value* | Significance |
| LIVRQNAC | 40 | 0.6283 | 0.0585 | 3 | *** | 0.0001 |
| LIVRQNAC | 30 | 0.5975 | 0.0709 | 3 | **** | 0.0001 |
| LIVRQNAC | 20 | 0.6504 | 0.0622 | 4 | *** | 0.0001 |
| LIVRQNAC | 10 | 0.8287 | 0.0936 | 4 | * | 0.0277 |
| LIVRQNAC | 1 | 1.0000 | 0.0908 | 4 | | |
| LIVRQNAC + G | 40 | 0.5288 | 0.0402 | 3 | *** | 0.0006 |
| LIVRQNAC + G | 30 | 0.6297 | 0.0200 | 3 | ** | 0.0042 |
| LIVRQNAC + G | 20 | 0.5926 | 0.0634 | 4 | ** | 0.001 |
| LIVRQNAC + G | 10 | 0.7404 | 0.0920 | 4 | * | 0.0267 |
| LIVRQNAC + G | 1 | 1.0000 | 0.2151 | 4 | | |
| LIVRQNAC + S | 40 | 0.5900 | 0.0450 | 3 | *** | 0.0003 |
| LIVRQNAC + S | 30 | 0.5562 | 0.1242 | 3 | *** | 0.0002 |
| LIVRQNAC + S | 20 | 0.6844 | 0.0638 | 3 | ** | 0.0022 |
| LIVRQNAC + S | 10 | 0.7003 | 0.0946 | 3 | ** | 0.0032 |
| LIVRQNAC + S | 1 | 1.0000 | 0.0311 | 3 | | |
| LIV | 40 | 1.3017 | 0.1474 | 3 | ns | 0.0518 |
| LIV | 30 | 1.3358 | 0.1922 | 3 | * | 0.0305 |
| LIV | 20 | 1.2592 | 0.0747 | 3 | ns | 0.0997 |
| LIV | 10 | 1.0149 | 0.1089 | 3 | ns | 0.9997 |
| LIV | 1 | 1.0000 | 0.0828 | 3 | | |
| LIVRQ | 40 | 1.0070 | 0.1716 | 3 | ns | 0.9999 |
| LIVRQ | 30 | 1.0190 | 0.1103 | 3 | ns | 0.9983 |
| LIVRQ | 20 | 1.1403 | 0.0516 | 3 | ns | 0.3875 |
| LIVRQ | 10 | 1.0454 | 0.0908 | 3 | ns | 0.9609 |
| LIVRQ | 1 | 1.0000 | 0.0935 | 3 | | |
| RQNAC | 40 | 0.3622 | 0.0166 | 3 | **** | 0.0001 |
| RQNAC | 30 | 0.4232 | 0.0819 | 3 | **** | 0.0001 |
| RQNAC | 20 | 0.5819 | 0.0574 | 3 | *** | 0.0001 |
| RQNAC | 10 | 0.8181 | 0.0703 | 3 | * | 0.0313 |
| RQNAC | 1 | 1.0000 | 0.0967 | 3 | | |
| N-Acetyl Cysteine | 40 | 0.5076 | 0.0154 | 3 | **** | 0.0001 |
| N-Acetyl Cysteine | 20 | 0.6593 | 0.0914 | 3 | *** | 0.0003 |
| N-Acetyl Cysteine | 10 | 0.7939 | 0.0715 | 3 | ** | 0.01 |
| N-Acetyl Cysteine | 5 | 0.9175 | 0.0519 | 3 | ns | 0.3855 |
| N-Acetyl Cysteine | 0 | 1.0000 | 0.0686 | 3 | | |

TABLE 33
Fold change of procollagen 1α1 secretion after administration of a single amino acid
composition in the first donor

| | | Procollagen Ia1 Secretion (Fold Change of 1X) | | | | |
|---|---|---|---|---|---|---|
| Amino Acid | Conc. | | Std. | Number | | |
| Supplement | (μM) | Mean | Deviation | of values | P-value* | Significance |
| Valine | 23420 | 1.2139 | 0.0544 | 3 | ns | 0.1392 |
| Valine | 11710 | 1.2069 | 0.0881 | 3 | ns | 0.155 |
| Valine | 4684 | 1.1203 | 0.1908 | 3 | ns | 0.5111 |
| Valine | 234 | 1.0000 | 0.1389 | 4 | | |
| Arginine | 5440 | 1.0646 | 0.0939 | 3 | ns | 0.4155 |
| Arginine | 2720 | 1.1757 | 0.0466 | 3 | * | 0.01 |
| Arginine | 1088 | 1.0291 | 0.0615 | 4 | ns | 0.8428 |
| Arginine | 109 | 1.0000 | 0.0389 | 4 | | |
| Glutamine | 22484 | 1.0564 | 0.1293 | 3 | ns | 0.8468 |
| Glutamine | 11242 | 1.0888 | 0.0261 | 3 | ns | 0.5648 |
| Glutamine | 3747 | 1.0757 | 0.1003 | 4 | ns | 0.6356 |
| Glutamine | 749 | 0.9790 | 0.0836 | 4 | ns | 0.993 |
| Glutamine | 562 | 1.0000 | 0.0596 | 3 | | |
| Isoleucine | 6639 | 1.2144 | 0.1129 | 3 | ns | 0.0537 |
| Isoleucine | 3320 | 1.1366 | 0.0938 | 3 | ns | 0.2411 |
| Isoleucine | 1328 | 0.9229 | 0.0614 | 3 | ns | 0.6321 |
| Isoleucine | 66 | 1.0000 | 0.0953 | 3 | | |
| Leucine | 15270 | 1.1710 | 0.1043 | 3 | ns | 0.094 |
| Leucine | 7635 | 1.0915 | 0.0832 | 3 | ns | 0.4736 |
| Leucine | 3054 | 1.1410 | 0.1245 | 4 | ns | 0.1424 |
| Leucine | 153 | 1.0000 | 0.0481 | 4 | | |

TABLE 33-1
Fold change of procollagen 1α1 secretion after administration of an amino
acid composition in the second donor

| | | Procollagen Ia1 Secretion (Fold Change of 1X) | | | | |
|---|---|---|---|---|---|---|
| | | | Std. | Number | | |
| Amino Acid Supplement | Conc. (X) | Mean | Deviation | of values | P-value* | Significance |
| LIVRQNAC | 40 | 0.7465 | 0.0551 | 3 | ** | 0.0041 |
| LIVRQNAC | 30 | 0.6829 | 0.0991 | 3 | *** | 0.0007 |
| LIVRQNAC | 20 | 0.6922 | 0.0281 | 4 | *** | 0.0004 |
| LIVRQNAC | 10 | 0.7879 | 0.0748 | 4 | ** | 0.0085 |
| LIVRQNAC | 1 | 1.0000 | 0.1141 | 4 | | |
| LIVRQNAC + G | 40 | 0.6372 | 0.0267 | 3 | **** | 0.0001 |
| LIVRQNAC + G | 30 | 0.7347 | 0.0324 | 3 | **** | 0.0001 |
| LIVRQNAC + G | 20 | 0.6716 | 0.0552 | 4 | **** | 0.0001 |
| LIVRQNAC + G | 10 | 0.7823 | 0.0579 | 4 | *** | 0.0001 |
| LIVRQNAC + G | 1 | 1.0000 | 0.0580 | 4 | | |
| LIVRQNAC + S | 40 | 0.8756 | 0.0372 | 3 | ns | 0.1229 |
| LIVRQNAC + S | 30 | 0.7340 | 0.0432 | 3 | ** | 0.0019 |
| LIVRQNAC + S | 20 | 0.7405 | 0.0491 | 3 | ** | 0.0022 |
| LIVRQNAC + S | 10 | 0.7472 | 0.0710 | 3 | ** | 0.0027 |
| LIVRQNAC + S | 1 | 1.0000 | 0.1031 | 3 | | |
| LIV | 40 | 1.4409 | 0.0697 | 3 | **** | 0.0001 |
| LIV | 30 | 1.3679 | 0.0156 | 3 | *** | 0.0001 |
| LIV | 20 | 1.3418 | 0.1090 | 3 | *** | 0.0002 |
| LIV | 10 | 1.2176 | 0.0343 | 3 | ** | 0.0057 |
| LIV | 1 | 1.0000 | 0.0396 | 3 | | |
| LIVRQ | 40 | 0.9851 | 0.0534 | 3 | ns | 0.9965 |
| LIVRQ | 30 | 1.0185 | 0.0735 | 3 | ns | 0.9921 |
| LIVRQ | 20 | 0.9212 | 0.0215 | 3 | ns | 0.4893 |
| LIVRQ | 10 | 0.9558 | 0.0580 | 3 | ns | 0.8556 |
| LIVRQ | 1 | 1.0000 | 0.1134 | 3 | | |
| RQNAC | 40 | 0.6363 | 0.0432 | 3 | *** | 0.0002 |
| RQNAC | 30 | 0.6154 | 0.0196 | 3 | *** | 0.0001 |
| RQNAC | 20 | 0.7060 | 0.0851 | 3 | *** | 0.0009 |
| RQNAC | 10 | 0.8385 | 0.0248 | 3 | * | 0.041 |
| RQNAC | 1 | 1.0000 | 0.1071 | 3 | | |
| N-Acetyl Cysteine | 40 | 0.8383 | 0.0378 | 3 | ns | 0.4053 |
| N-Acetyl Cysteine | 20 | 0.7378 | 0.1347 | 3 | ns | 0.1002 |
| N-Acetyl Cysteine | 10 | 0.8877 | 0.2282 | 3 | ns | 0.6842 |
| N-Acetyl Cysteine | 5 | 0.8387 | 0.0832 | 3 | ns | 0.407 |
| N-Acetyl Cysteine | 0 | 1.0000 | 0.0808 | 3 | | |

TABLE 33-2
Fold change of procollagen 1α1 secretion after administration of a single amino acid
composition in the second donor

| | | Procollagen Ia1 Secretion (Fold Change of 1X) | | | | |
|---|---|---|---|---|---|---|
| Amino Acid | Conc. | | Std. | Number | | |
| Supplement | (μM) | Mean | Deviation | of values | P-value* | Significance |
| Valine | 23420 | 1.3068 | 0.0963 | 3 | ** | 0.0019 |
| Valine | 11710 | 1.2877 | 0.1122 | 3 | ** | 0.0029 |
| Valine | 4684 | 1.2865 | 0.0717 | 4 | ** | 0.0018 |
| Valine | 234 | 1.0000 | 0.0589 | 4 | | |
| Arginine | 5440 | 1.1304 | 0.0187 | 3 | ns | 0.0937 |
| Arginine | 2720 | 1.0722 | 0.0791 | 3 | ns | 0.4483 |
| Arginine | 1088 | 1.0126 | 0.0822 | 4 | ns | 0.989 |
| Arginine | 109 | 1.0000 | 0.0778 | 4 | | |
| Glutamine | 22484 | 0.7143 | 0.0566 | 3 | ** | 0.0058 |
| Glutamine | 11242 | 0.7080 | 0.0246 | 3 | ** | 0.005 |
| Glutamine | 3747 | 0.7541 | 0.0860 | 4 | * | 0.0102 |
| Glutamine | 749 | 0.9191 | 0.1171 | 4 | ns | 0.5776 |
| Glutamine | 562 | 1.0000 | 0.1003 | 3 | | |
| Isoleucine | 6639 | 1.5423 | 0.1489 | 3 | ** | 0.006 |
| Isoleucine | 3320 | 1.4940 | 0.0238 | 3 | * | 0.0102 |
| Isoleucine | 1328 | 1.4811 | 0.2307 | 3 | * | 0.0117 |
| Isoleucine | 66 | 1.0000 | 0.1264 | 3 | | |
| Leucine | 15270 | 0.9518 | 0.0406 | 3 | ns | 0.9292 |
| Leucine | 7635 | 1.2628 | 0.1763 | 3 | ns | 0.0607 |
| Leucine | 3054 | 1.0781 | 0.1735 | 4 | ns | 0.7374 |
| Leucine | 153 | 1.0000 | 0.0681 | 4 | | |

TABLE 33-3
Fold change of procollagen 1α1 secretion after administration of an amino
acid composition in the third donor

| | | Procollagen Ia1 Secretion (Fold Change of 1X) | | | | |
|---|---|---|---|---|---|---|
| | | | Std. | Number | | |
| Amino Acid Supplement | Conc. (X) | Mean | Deviation | of values | P-value* | Significance |
| LIVRQNAC | 40 | 0.9052 | 0.0344 | 3 | ns | 0.5685 |
| LIVRQNAC | 30 | 0.7456 | 0.0895 | 3 | * | 0.0192 |
| LIVRQNAC | 20 | 0.7817 | 0.0680 | 4 | * | 0.03 |
| LIVRQNAC | 10 | 0.9774 | 0.1451 | 4 | ns | 0.9927 |
| LIVRQNAC | 1 | 1.0000 | 0.1116 | 4 | | |
| LIVRQNAC + G | 40 | 0.7040 | 0.0080 | 3 | ** | 0.002 |
| LIVRQNAC + G | 30 | 0.6249 | 0.0819 | 3 | *** | 0.0003 |
| LIVRQNAC + G | 20 | 0.6863 | 0.1334 | 4 | *** | 0.0006 |
| LIVRQNAC + G | 10 | 1.0068 | 0.0642 | 4 | ns | 0.9998 |
| LIVRQNAC + G | 1 | 1.0000 | 0.0724 | 4 | | |
| LIVRQNAC + S | 40 | 0.9190 | 0.0772 | 3 | ns | 0.3351 |
| LIVRQNAC + S | 30 | 0.8107 | 0.0596 | 3 | * | 0.0101 |
| LIVRQNAC + S | 20 | 0.8878 | 0.0129 | 3 | ns | 0.1296 |
| LIVRQNAC + S | 10 | 0.9814 | 0.0458 | 3 | ns | 0.9852 |
| LIVRQNAC + S | 1 | 1.0000 | 0.0780 | 3 | | |
| LIV | 40 | 1.3233 | 0.0667 | 3 | ** | 0.0024 |
| LIV | 30 | 1.2510 | 0.1070 | 3 | * | 0.0125 |
| LIV | 20 | 1.2702 | 0.0639 | 3 | ** | 0.0079 |
| LIV | 10 | 1.1912 | 0.1049 | 3 | ns | 0.0532 |
| LIV | 1 | 1.0000 | 0.0521 | 3 | | |
| LIVRQ | 40 | 1.2020 | 0.1119 | 3 | ns | 0.1081 |
| LIVRQ | 30 | 1.1380 | 0.0955 | 3 | ns | 0.3407 |
| LIVRQ | 20 | 0.9489 | 0.1179 | 3 | ns | 0.9263 |
| LIVRQ | 10 | 1.0786 | 0.0764 | 3 | ns | 0.7564 |
| LIVRQ | 1 | 1.0000 | 0.1056 | 3 | | |
| RQNAC | 40 | 0.6590 | 0.0860 | 3 | ** | 0.0012 |
| RQNAC | 30 | 0.6708 | 0.0407 | 3 | ** | 0.0016 |
| RQNAC | 20 | 0.9135 | 0.1192 | 3 | ns | 0.5063 |
| RQNAC | 10 | 0.8783 | 0.0515 | 3 | ns | 0.245 |
| RQNAC | 1 | 1.0000 | 0.0740 | 3 | | |
| N-Acetyl Cysteine | 40 | 0.6962 | 0.0189 | 3 | * | 0.0125 |
| N-Acetyl Cysteine | 20 | 0.8521 | 0.0709 | 3 | ns | 0.2666 |
| N-Acetyl Cysteine | 10 | 0.9391 | 0.1250 | 3 | ns | 0.8641 |
| N-Acetyl Cysteine | 5 | 1.0897 | 0.1245 | 3 | ns | 0.6511 |
| N-Acetyl Cysteine | 0 | 1.0000 | 0.1133 | 3 | | |

TABLE 33-4
Fold change of procollagen 1α1 secretion after administration of a single amino acid
composition in the third donor

| | | Procollagen Ia1 Secretion (Fold Change of 1X) | | | | |
|---|---|---|---|---|---|---|
| Amino Acid | | | Std. | Number | | |
| Supplement | Conc. (μM) | Mean | Deviation | of values | P-value* | Significance |
| Valine | 23420 | 1.1139 | 0.1077 | 3 | ns | 0.5315 |
| Valine | 11710 | 1.0498 | 0.1773 | 3 | ns | 0.918 |
| Valine | 4684 | 1.0428 | 0.1036 | 4 | ns | 0.9323 |
| Valine | 234 | 1.0000 | 0.1203 | 4 | | |
| Arginine | 5440 | 1.2125 | 0.0862 | 3 | * | 0.0112 |
| Arginine | 2720 | 1.1314 | 0.0820 | 3 | ns | 0.1114 |
| Arginine | 1088 | 1.0623 | 0.0629 | 4 | ns | 0.5378 |
| Arginine | 109 | 1.0000 | 0.0760 | 4 | | |
| Glutamine | 22484 | 1.0121 | 0.0730 | 3 | ns | 0.9989 |
| Glutamine | 11242 | 1.1204 | 0.1056 | 3 | ns | 0.2356 |
| Glutamine | 3747 | 0.9734 | 0.0900 | 4 | ns | 0.9747 |
| Glutamine | 749 | 1.0317 | 0.0644 | 4 | ns | 0.9538 |
| Glutamine | 562 | 1.0000 | 0.0447 | 3 | | |
| Isoleucine | 6639 | 1.4465 | 0.0958 | 3 | ** | 0.0014 |
| Isoleucine | 3320 | 1.2703 | 0.0352 | 3 | * | 0.024 |
| Isoleucine | 1328 | 1.2687 | 0.0374 | 3 | * | 0.0247 |
| Isoleucine | 66 | 1.0000 | 0.1629 | 3 | | |
| Leucine | 15270 | 0.9892 | 0.0260 | 3 | ns | 0.9979 |
| Leucine | 7635 | 1.2027 | 0.0693 | 3 | ns | 0.0638 |
| Leucine | 3054 | 1.1399 | 0.1385 | 4 | ns | 0.1844 |
| Leucine | 153 | 1.0000 | 0.1077 | 4 | | |

### Example 6. Treatment of NASH in a Mouse Model with an Amino Acid Composition Induction of NASH in Mice

In one example, the effects of LIVRQNAC and related amino acid compositions in the obesity, metabolism-driven non-alcoholic steatohepatitis (NASH) in FATZO mouse model was examined.

NASH was induced in 60 male FATZO mice by a western diet (Research Diet #D12079B; fat 40% kcal, protein 17% kcal, carbohydrate 43% kcal) supplemented with 5% fructose in the drinking water (WDF) during a 16 week induction phase. Diets and water were available ad libitum. Littermate control male FATZO mice fed with a control diet (n=6, Purina #5008; fat 17% kcal, protein 27% kcal, carbohydrate 56% kcal) and sterile water were set up for control purpose. Mice were housed in plastic cages with microisolator. Sterilized bedding was replaced once a week. Mice were housed three per cage and maintained on a twelve hour light cycle throughout study duration. Room temperature was monitored daily and maintained at 22-25° C. Body weight was recorded every week during the induction phase.

Following 16 weeks diet induction, 6 mice remained on control diet (group 1, Control) while 60 induced mice were randomized on body weight and plasma glucose (fed) for assignment to the following treatments. FATZO mice were administered with test articles starting at 16 weeks post western diet NASH induction for 4 weeks. Test articles were administered by oral gavage. Animals were euthanized at 20 weeks post western diet NASH induction, and tissues were harvested for analysis.

| Group | (n) | Treatment (oral) | Diet |
|---|---|---|---|
| 1 | 6 | Vehicle | 5008 WDF |
| 2 | 10 | Vehicle | D12079B + 5% Fructose |
| 3 | 10 | LIVRQNAC (1500 mg/kg) | D12079B + 5% Fructose |
| 4 | 10 | LIVRQNAC (3000 mg/kg) | D12079B + 5% Fructose |
| 5 | 10 | LIVRQNAC + G (3885 mg/kg) | D12079B + 5% Fructose |
| 6 | 10 | LRQNAC (2469 mg/kg) | D12079B + 5% Fructose |
| 7 | 10 | OCA (30 mg/kg/day) | D12079B + 5% Fructose |

LIVRQNAC, LIVRQNAC+G, LRQNAC, and OCA (Advanced ChemBlocks, Inc.), incipient, and water for irrigation were provided by Axcella Health, Inc. 0.5% Methylcellulose was provided by CrownBio, Inc. Dosing solutions were prepared according to Appendix 1. TA compounds (amino acid compositions) were amino acid blends formulated fresh daily in water for irrigation (Baxter #27F7114) and the excipients 0.125% Xanthan Gum, 1.5 mM Sodium Lauryl Sulfate and 0.28% Lecithin. Obeticholic acid (OCA) was suspended in 0.5% methylcellulose in water for irrigation. All test articles were stored refrigerated. TA compounds were provided in frozen powder form by the sponsor. Dosing was continued for 4 weeks.

Leucine dosages of LIVRQNAC+G and LRQNAC were matched to that of LIVRQNAC.

| | | | Supplier Part | Lot |
|---|---|---|---|---|
| Ingredient | Grade | Supplier | Number | Number |
| Fusi-BCAA | Instantized | Ajinomoto | 33555 | OH704 |
| Unflavored | (0.3-0.9% | (AjiPure) | | |
| (2:1:1 L-Leu: | Lecithin) | | | |
| L-Ile:L-Val) | | | | |
| L-Arginine HCl | USP | Sigma | A4599 | CDB0352V |
| | | (Ajinomoto) | | |
| L-Arginine HCl | USP | Sigma | A4599 | CDB0352V |
| | | (Ajinomoto) | | |
| L-Glutamine | USP | Ajinomoto | 32824 | R014A003 |
| Glycine | USP | Ajinomoto | 30359 | R015T008 |
| Acetylcysteine | USP | Spectrum | AC126 | 1FI0576 |
| (NAC) | | Chemical | | |

| | | LIVRQNAC + | |
|---|---|---|---|
| | LIVRQNAC | G | LRQNAC |
| | Daily | Daily | Daily |
| Ingredient | Dose (g) | Dose (g) | Dose (g) |
| Fusi-BCAA Unflavored | 24.0 | 24.0 | |
| (2:1:1 L-Leu:L-Ile:L-Val) | | | |
| Fusil (L-Leucine) | | | 12.0 |
| L-Arginine HCl | 18.0 | 18.0 | 18.0 |
| L-Glutamine | 24.0 | 24.0 | 24.0 |
| Glycine | | 20.0 | |
| Acetylcysteine (NAC) | 1.8 | 1.8 | 1.8 |
| AMINO ACIDS = | 67.8 | 87.8 | 55.8 |

LIVRQNAC, LIVRQNAC+G, LRQNAC, OCA and Vehicle were administered by oral gavage at a volume of 10 mL/kg throughout the study. Dosages were calculated by daily body weight. LIVRQNAC, LIVRQNAC+G, LRQNAC, and Vehicle were administered twice per day (BID), while OCA was administered once a day (QD) in the morning. Mice receiving OCA once per day (QD), and one vehicle QD. Doses were administered by oral gavage at 0700 and 1800 by oral gavage for 4 weeks.

The viability, clinical signs and behavior were monitored daily. Body weight was recorded daily during the dosing period. Blood samples were collected weekly in the AM (0700) via tail clip for glucose measurement (StatStrip glucometer).

Animals were anesthetized with CO2 inhalation and exsanguinated via cardiac puncture for euthanasia. Terminal blood samples (K2EDTA) were obtained by cardiac puncture in anesthetized animals at termination. Samples were provided frozen to Axcella Health. Organ weights (total liver, gonadal fat pads) were recorded. Pancreas, and small intestine and gonadal fat pads were fixed in 10% Buffered Formalin and prepared as directed in protocol. A section of small intestine, gonadal fat pad and liver were also snap frozen in liquid nitrogen and shipped to the sponsor.

The liver tissues were fixed in Bouin's solution at 4° C. for 24 hours followed by baths of standard concentrations of alcohol then xylene to prepare the tissues for paraffin embedding. After being embedded in paraffin and cooled, five-micron sections were cut and stained for routine H&E and Picric Sirius Red. A section of both right and left lobes of the livers were frozen in OCT for analysis of lipid content with Oil-Red-) staining. The Aperio whole slide digital imaging system (Scan Scope CS, Vista, Calif.) was used for imaging. All slides were imaged at 20×. The scan time ranged from 1.5 minutes to a maximum time of 2.25 minutes. The whole images were housed and stored in their Spectrum software system and images were shot from the whole slides.

The livers were evaluated using the NASH liver criteria for scoring. In this mouse study, one cross section of liver for each case was analyzed with the NASH score system. According to the published NASH CRN Scoring System, this scoring system comprises of NAFLD Activity Score (NAS), fibrosis stage and identification of NASH by pattern recognition. The NAS can range from 0 to 8 and is calculated by the sum of scores of steatosis (0-3), lobular inflammation (0-3) and hepatocyte ballooning (0-2) from H&E stained sections. Fibrosis was scored (0-4) from picrosirius red stained slides. The NASH system is used for human liver 18 gauge biopsies. Steatosis, lobular inflammation, hepatocyte. balloon degeneration, fibrosis, NAS and the presence of NASH by pattern recognition were systematically assessed. In this study we evaluated one total cross section of liver per mouse in this study. This is about 15 times the size of an 18 gauge human liver biopsy. The pathology score was determined as 0, +1, +2, or +3. The lesions were scored on location (periportal, centrilobular, and mid zonal) and fat accumulation (focal, periportal, and/or centrilobular). The other part of the score was distribution of the lesions: focal, multifocal and/or diffuse. Also, mild, moderate and severity of the lesions. These parameters made up the total NASH score.

All immunohistochemical staining steps were performed using the Dako FLEX SYSTEM on an automated immunostainer; incubations were done at room temperature and Tris buffered saline plus 0.05% Tween 20, pH 7.4 (TBS—Dako Corp.) was used for all washes and diluents. Thorough washing was performed after each incubation. Primary antibodies included anti-mouse SMA, F4/80, Mac-2, and Picric Sirius Red. Control sections were treated with an isotype control using the same concentration as primary antibodies to verify the staining specificity.

White adipose tissue (WAT) adipocyte size was analyzed from the H&E stained sections. Using the Aperio Image Scope application, 3 localized regions (edge of tissue, tissue not surrounding vascular area, tissue surrounding vascular area) of each tissue specimen were assessed by measuring the area of 10 largest adipocytes of the region. Within each tissue, 10 hot spots of each regions were quantified (um²) and averaged.

Pancreatic beta-islet cells were identified by immunohistochemical staining.

Aperio Automatic Image Quantitation was employed to quantify positive pixels of immunohistochemical staining, Oil-Red 0, and Sirius Red staining. The Positive Pixel Count algorithm was used to quantify the percentage of a specific stain present in a scanned slide image. A range of color (range of hues and saturation) and three intensity ranges (weak, positive, and strong) were masked and evaluated. The algorithm counted the number and intensity-sum in each intensity range, along with three additional quantities: average intensity, ratio of strong/total number, and average intensity of weak positive pixels. The positive pixel algorithm was modified to distinguish between the orange and blue colors. Alterations from the normal “hue value” (0.1 to 0.96) and “color saturation” (0.04 to 0.29), were made for the Sirius Red evaluation. Vasculature and artifacts were excluded from analysis.

Liver IL-1b protein level was quantified using the multiplex ELISA Assay (Meso Scale Discovery, Rockville, Md.).

Statistical analyses of liver histological scores were performed using Bonferroni Multiple Comparison Test on GraphPad Prism 6 (GraphPad Software Inc., USA). P values <0.05 were considered statistically significant. Results were expressed as mean±SEM. Comparisons were made between Group 2 (Vehicle) and the following groups; Group 3 (LIVRQNAC 1,500 mg/kg), Group 4 (LIVRQNAC 3,000 mg/kg), Group 5 (LIVRQNAC+G, 3,885 mg/kg), and (LRQNAC, 2,469 mg/kg).

### Body and Liver Weight

Feeding the western diet supplemented with fructose (WDF) for 16 weeks elicited significant effects on body weight compared to control fed animals. Prior to administration of test agent, animals fed the WDF were significantly heavier (47.6±0.45 vs. 43.9±1.03 g; p<0.01) compared to animals fed the control diet.

Body weight decreased compared to baseline values in all treatment groups; there were no significant differences in weight loss compared to vehicle (−7.6±0.9, −6.9±1.3, −6.8±1.4, −5.7±1.2, −6.4±1.0, −4.7±1.6 and −3.9±1.5% for control, vehicle, LIVRQNAC (1500 mg/kg), LIVRQNAC (3000 mg/kg), LIVRQNAC+G, LRQNAC, and OCA, respectively; p<0.4992).

Liver weight (% body weight) was significantly higher in vehicle treated animals fed WDF compared to control diet (7.22±0.3 vs. 5.05±0.24%; p<0.0001); however, in animals fed WDF, no significant effects compared to vehicle were noted in any treatment group (7.22±03, 7.14±0.3, 7.19±0.26, 6.69±0.18, 7.02±0.5 and 6.81±0.2 for vehicle, LIVRQNAC (1500 mg/kg), LIVRQNAC (3000 mg/kg), LIVRQNAC+G, LRQNAC, and OCA, respectively; p<0.7450).

### Liver Histology

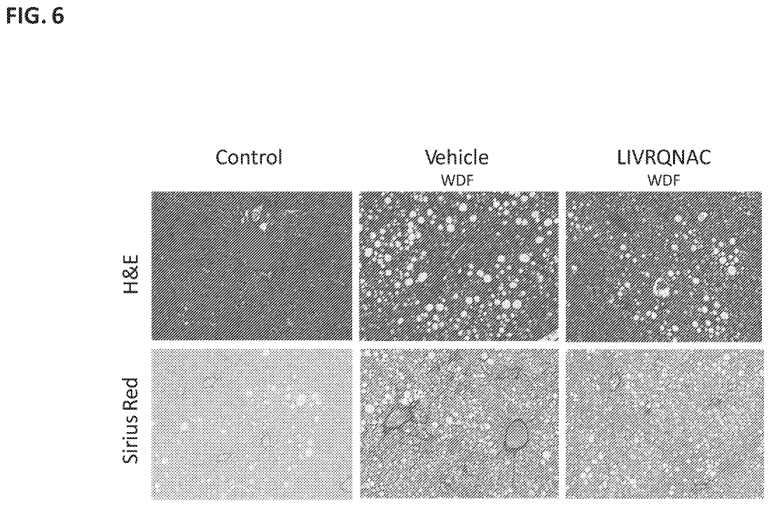

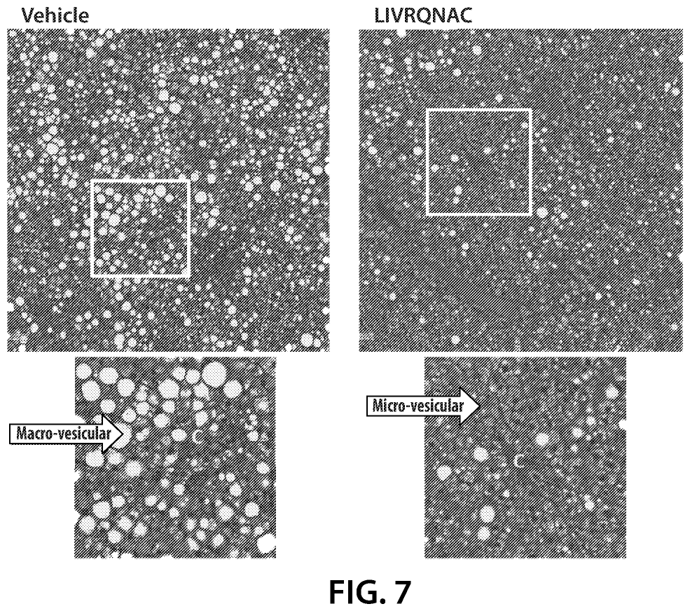

FATZO mice fed with the control diet developed mild steatosis, ballooning, or fibrosis (FIG. 6). FATZO mice fed with the WDF and treated with vehicle developed significant steatosis, ballooning, and fibrosis. In contrast to predominantly macrovesicular steatosis in the vehicle groups, a mixture of predominantly microvesicular and diminished macrovesicular steatosis was observed in LIVRQNAC, LIVRQNAC+G and LRQNAC groups, as shown in FIG. 7.

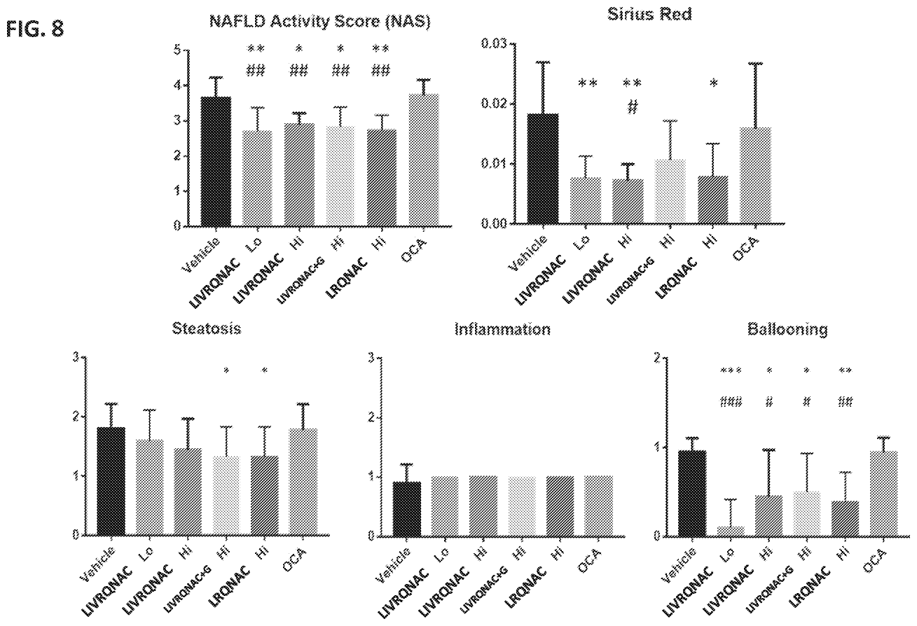

The NAFLD activity score is calculated from histological scoring of steatosis (0-3) and ballooning (0-2) in fixed liver tissues. In WDF fed animals, all amino acid composition treatments produced a significant reduction in the NAS compared to the vehicle treatment group (FIG. 8). LIVRQNAC and amino acid composition treatments reduced liver steatosis as compared to vehicle, although only LIVRQNAC+G and LRQNAC reached statistical significance (p<0.05), while LIVRQNAC did not (LIVRQNAC 3.0 g/kg, p=0.12). All amino acid composition treatments significantly attenuated hepatocyte ballooning, the biomarker of lipotoxicity and cell death. In conclusion, amino acid composition-associated improvement of liver pathology is mainly attributed to attenuation of hepatocyte ballooning. There was no significant effect of OCA on the NAS score and NAS components compared to vehicle.

| Liver | | LIVRQNAC | LIVRQNAC | | | |
|---|---|---|---|---|---|---|
| Pathology | Vehicle | 1.5 g/kg | 3.0 g/kg | LIVRQNAC + G | LRQNAC | OCA |
| NAS | 3.65 ± 0.183 | 2.70 ± 0.213 | 2.89 ± 0.111 | 2.83 ± 0.186 | 2.72 ± 0.147 | 3.72 ± 0.147 |
| Steatosis | 1.8 ± 0.133 | 1.6 ± 0.163 | 1.44 ± 0.176 | 1.33 ± 0.167 | 1.33 ± 0.167 | 1.78 ± 0.147 |
| Inflammation | 0.9 ± 0.1 | 1.0 ± 0.0 | 1.0 ± 0.0 | 1.0 ± 0.0 | 1.0 ± 0.0 | 1.0 ± 0.0 |
| Ballooning | 0.95 ± 0.05 | 0.1 ± 0.1 | 0.44 ± 0.176 | 0.50 ± 0.144 | 0.39 ± 0.111 | 0.94 ± 0.056 |

Livers from vehicle treated animals demonstrated a mild fibrosis; score of 0.8±0.1. Only livers from animals treated with LIVRQNAC (1500 mg/kg) demonstrated a significant reduction in fibrosis when compared to the vehicle treated group, (0.2±0.1 versus 0.8±0.1, p<0.01), but not with LIVRQNAC (3000 mg/kg), LIVRQNAC+G or LRQNAC. Sirius Red collagen staining demonstrated that all amino acid composition treatments had significantly lower collagen deposition compared to vehicle (LIVRQNAC 1500 mg/kg, p<0.01; LIVRQNAC 3000 mg/kg, p<0.01; LIVRQNAC+G, p=0.09; LRQNAC, p<0.05). OCA did not affect liver fibrosis score or Sirius Red collagen staining area.

| | | LIVRQNAC | LIVRQNAC | | | |
|---|---|---|---|---|---|---|
| Fibrosis | Vehicle | 1.5 g/kg | 3.0 g/kg | LIVRQNAC + G | LRQNAC | OCA |
| Fibrosis | 0.8 ± 0.133 | 0.2 ± 0.133 | 0.44 ± 0.176 | 0.44 ± 0.176 | 0.33 ± 0.167 | 0.67 ± 0.167 |
| Sirius | 1.82 ± 0.279 | 0.77 ± 0.116 | 0.72 ± 0.092 | 0.107 ± 0.218 | 0.79 ± 0.183 | 1.59 ± 0.36 |
| Red | | | | | | |

### Liver Chemokines and Cytokines

Proinflammatory cytokine IL-1b protein level in liver was elevated in the WDF fed mice as compared to control diet-fed mice, as shown in Table 34.

TABLE 34
Mean liver IL-1b protein levels after administration
of amino acid compositions

| | | | LIVRQNAC | LIVRQNAC + | LRQNAC |
|---|---|---|---|---|---|
| | Ctrl | | (3000 | G (3885 | (2469 |
| IL-1b | diet | Vehicle | mg/kg) | mg/kg) | mg/kg) |
| Mean | 10.53 | 22.31 | 22.11 | 14.42 | 28.85 |
| SEM | 1.248 | 6.063 | 5.739 | 3.299 | 10.41 |

### Summary

Based on clinical observations, WDF-fed FATZO mice gained more body weight that those fed with a control diet. All treatments were well tolerated in FATZO mice. Both WDF-fed and control diet-fed mice lose body weight during the treatment period, which may be due to the stress associated with administration of test articles or vehicle via oral gavage twice a day.

NAS was significantly attenuated in all amino acid composition treatment groups as compared to vehicle, predominantly attributing to ballooning score. Hepatocyte ballooning was significantly reduced in all the amino acid composition treatment groups. Steatosis was significantly reduced in LIVRQNAC+G and LRQNAC treatment groups. LIVRQNAC also lowered steatosis, although the difference was not significant. Consistent with the histological and biochemical data, de novo lipogenesis enzymes FASN and ACACA RNA levels were not affected by amino acid composition treatment.

The characteristics of hepatocyte steatosis were differed by amino acid composition treatments. Liver of the WDF-fed mice (vehicle group) demonstrated predominantly macrovesicular steatosis. In contrast, macrovesicular steatosis was diminished, and a mixture of microvesicular and macrovesicular steatosis in all amino acid composition treatment groups. The biological meaning and mechanism of amino acid compositions on macro- to microvesicular steatosis phenotypes merit further investigation.

Liver fibrosis score in FATZO model of NAFLD was significantly attenuated by LIVRQNAC treatment at low dose but not at high dose. LIVRQNAC+G and LRQNAC had no effect on fibrosis. Nonetheless, Sirius Red collagen staining demonstrated that LIVRQNAC, LIVRQNAC+G and LRQNAC significantly reduced collagen deposition in the liver.

In conclusion, all three amino acid compositions (LIVRQNAC, LIVRQNAC+G and LRQNAC) tested in FATZO mice attenuate NAFLD activity scores, hepatocyte ballooning, and fibrosis. These amino acid compositions can be used to treat NASH. Glycine-containing amino acid compositions can further reduce pathways which results in reduced liver fibrosis

### Example 7. Treatment of Subjects with an Amino Acid Composition

The study described herein features the administration of a composition including amino acids to subjects with type 2 diabetes mellitus (T2DM) and nonalcoholic fatty liver disease (NAFLD). The goal of this pre-IND and IRB approved study was to determine the safety and tolerability of an amino acid composition as well as its impact on the structure and function of human physiology by looking at various markers of fibrosis, inflammation, insulin sensitivity, glucose and lipid metabolism, and apoptosis, after 6 weeks and 12 weeks of administration. The composition included about 1 g of L-leucine, about 0.5 g of L-isoleucine, about 0.5 g of L-valine, about 1.5 g of L-arginine (or 1.81 g of L-arginine HCl), about 2.0 g of L-glutamine, and about 0.15 g of N-acetylcysteine per stick packet, for administration in four stick packs three times per day (e.g., a total of about 72 g per day, or about 24 g three times per day).

In this study, subjects received the amino acid composition three times daily for 12 weeks. Amino acids were provided in powder form to be dissolved in 12 oz. of water. Participants were given the amino acid composition for the 12 week study period.

The primary outcome measure of this study was safety and tolerability. The secondary outcome measures were to examine the impact on human physiology through biomarkers that pertain to metabolism, inflammation and fibrosis. Assessments were performed at baseline (day 1), at week 6, and at week 12 of the study.

Key criteria for selecting subjects included the following: Men or women aged 18 to 70 years, inclusive; Willing and able to provide written informed consent; History of T2DM or Hemoglobin A1c (HbA1c) ≥6.5% and <10% at Screening; Documentation of fatty liver disease by one of the following criteria: a. Prior history of steatosis confirmed within 3 months of Screening by at least one of the following methods: Liver fat by MRI with a PDFF ≥8%; Fibroscan with Control Attenuation Parameter ≥300 dB/m; Liver biopsy indicating non-NASH NAFLD steatosis >Grade I. If the patient does not have this documented prior history of steatosis within 3 months of Screening (as noted in 4a), then a liver fat score of ≥10% must be documented at the time of Screening using the following formula:

Predicted percent liver fat=10\hat{ }(−0.805+(0.282*metabolic syndrome [yes=1/no=0])+(0.078*type 2 diabetes [yes=2/no=0])+(0.525*log 10(insulin mU/L))+(0.521*log 10(AST U/L))−(0.454*log 10(AST/ALT))34

Note: insulin, ALT and AST should be measured in a fasted serum sample. Subjects must be on stable exercise, diet and lifestyle routine within 3 months prior to Screening, with no major body weight fluctuations, i.e. subjects should be within ±3% of their body weight over the last 3 months at the time of Screening. Body mass index (BMI) ≥32 kg/m2 at Screening. For sites whose MRI equipment cannot accommodate a patient with a BMI of ≥45 kg/m2, an upper limit between 40 to 45 kg/m2 may be applied. Patients must be on a stable dose of glucose-lowering medication (which can include metformin, sulfonylureas, dipeptidyl peptidase-4 [DPP-4] inhibitors, sodium-glucose co-transporter 2 [SGLT2] inhibitors, or long-acting basal insulin) for at least 3 months before Screening and plan to remain on the same medication without anticipated dose adjustments of their medications for the duration of the study. See Section 8 below for a full list of excluded diabetes related medications. Subjects may be included in the study if they are concurrently treated with anti-hypertensive medications (e.g., beta blockers, hydrochlorothiazide, ACE inhibitors, angiotensin receptor blockers), medications for dyslipidemia (e.g., statins, fibrates), and medication for hypothyroidism (e.g., levothyroxine), so long as they have been on stable doses and regimen of these medications for at least 3 months before Screening and plan to remain on the same medication without anticipated dose adjustments of their medications for the duration of the study. Subjects may be on vitamin supplements (e.g. multivitamins; vitamin E <400 IU/day). However, they must be on stable doses and regimen of these vitamin supplements for at least 3 months before Screening without anticipated dose adjustments for the duration of the study. Female subjects of childbearing potential must have a negative serum pregnancy test at Screening and must agree and use a highly effective method of contraception during heterosexual intercourse during the entire study period and for 30 days following the last dose of study treatment. Childbearing potential refers to those female subjects who have not had a hysterectomy, bilateral oophorectomy, or medically-documented ovarian failure, or women <50 years of age with amenorrhea of any duration.

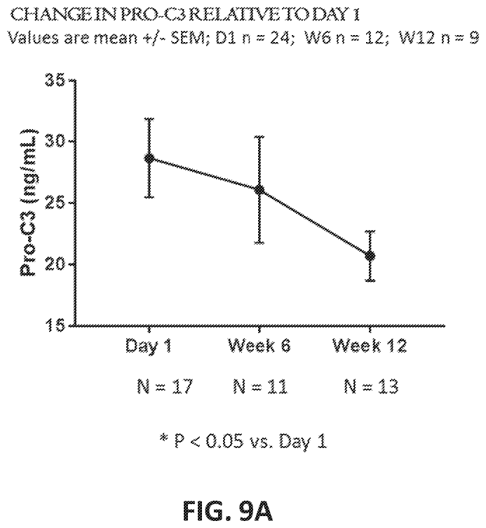

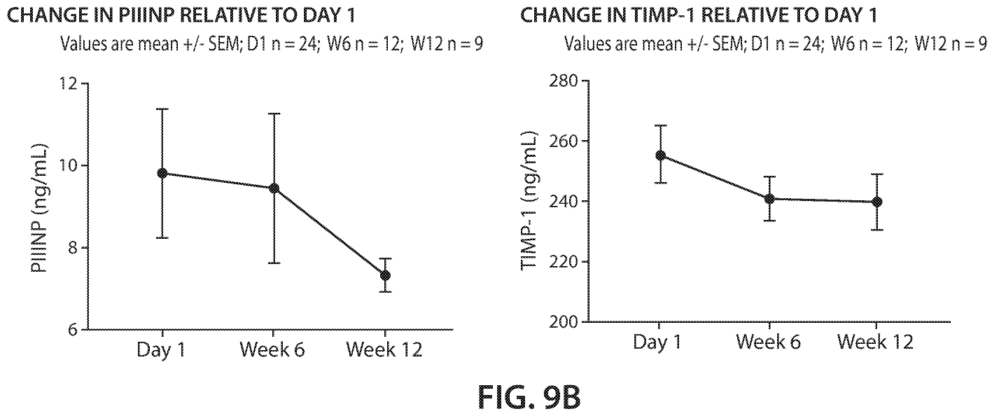

LIVRQNAC decreases plasma pro-C3 and other key fibrosis biomarkers at week 12, supporting a suppression of fibrogenesis. Mean levels of plasma proC3, PIIINP and TIMP-1 were determined at baseline (day 1) and at weeks 6 and 12. FIG. 9A shows average (in ng/ml, +/−SEM) of Pro-C3 over time, in the indicated number of subjects. LIVRQNAC significantly (p<0.05) decreased pro-C3 levels at week 12 compared to day 1. FIG. 9B shows that LIVRQNAC tends to decrease PIIINP and TIMP-1 levels (in ng/ml, +/−SEM) at weeks 6 and 12 relative to day 1.

The findings from this study suggest that the amino acid composition has a favorable safety and tolerability profile and impacts biomarkers for the structure and function of the human body that relate to fibrosis.

### Example 8: TGFβ1 Fibrogenic Gene Expression of Hepatic Stellate Cell

Primary human hepatic stellate cells were obtained from Samsara Sciences based on the following criteria for selecting donors: adult age (between 18 and 50 years), normal BMI (>18.5 and <25), and absence of confounding liver disease. Cells grown in Complete HSC Medium to ˜80% confluence in T75 or T150 flasks below passage 10 were seeded into sterile, collagen I coated, 96-well optical plastic microplates (ThermoScientific, 152036) at 6000 cells per well (1250 cells per cm2) and incubated overnight at 37° C., 5% CO2 in a humidified incubator in DMEM with 2% Fetal Bovine Serum and 1% Antibiotic-Antimycotic.

After the overnight incubation, plates were removed from the incubator and the medium was gently pipetted off and washed twice with 150 μL per well DPBS. The DPBS was removed and the pretreatment medium (±single amino acid dropout, 1×HMDB DMEM+1% Antibiotic-Antimycotic, 10 mM HEPES, ±supplemental amino acid dose; see experiment for medium composition) was applied to the cells at 150 μL per well. Plates were returned to the incubator for 10.5 hours.

After 10.5 hour pretreatment, the medium was removed from the cells, and the same pretreatment medium, now supplemented with 3 ng/mL TGFβ1, was applied. Each plate contained 3 ng/mL TGFβ1 in 1× human plasma amino acid (HMDB or PAA) concentration medium, 0 ng/mL in 1×HMDB, and 3 ng/mL TGFβ1+20 μM Silybin in 1×HMDB to serve as controls. Plates were then incubated for 24 hours at 37° C., 5% CO2.

After 24 hour stimulus, supernatant was removed and frozen at −80° C. in two separate aliquots. The cells were then washed with 125 μL per well Buffer FCW (FastLane Cell Multiplex NR Kit, Qiagen, 216713). The wash buffer was immediately removed and 50 μL of Cell Processing Mix (containing genomic DNA Wipeout buffer) was applied to lyse cells, incubating for 10 minutes at room temperature. RNA lysate was then transferred to 96-well qPCR plates, sealed, and gDNA was digested on thermal cycler at 75° C. for 5 minutes. RNA lysate was frozen at −80° C.

Each 20 μL one-step RT-qPCR reaction contained 4 μL of RNA lysate. Gene expression of Hsp47, and Gapdh were multiplexed using the HEX, and FAM fluorescent channels, respectively, with commercially available primer-probe mixes (the Human Hsp47 Primer-Probe Set, HEX; and the Human Gapdh Primer-Probe Set, FAM from IDT). Gene expression was evaluated using the ΔΔCq method within each single amino acid dropout and supplementation by normalizing to its own 1×HMDB concentration.

### Results

### Hsp47 Gene Expression

Tables 35, 36, 37, 38, 39, and 40 show the mean fold change in Hsp47 gene expression in primary human hepatic stellate cells from three different healthy donors. LIVRQNac, LIVRQNacG, LIVRQNacS, RQNac, and N-acetylcysteine decreased Hsp47 gene expression in all three donors. LIVRQ decreased Hsp47 in only one of three donors, and LIV had no significant impact on Hsp47 gene expression.

Leucine, isoleucine, and valine did not significantly change Hsp47 gene expression in any donor when the amino acid was administered alone. Arginine significantly increased Hsp47 gene expression in two of three donors when the amino acid was administered alone. Glutamine significantly increased Hsp47 gene expression in one of three donors when administered alone. N-acetyl cysteine significantly reduced Hsp47 gene expression in all three donors.

TABLE 35
Fold change of Hsp47 gene expression after administration of an amino acid composition,
normalized to Gapdh expression in a first donor

| | | Hsp47 Fold Expression Relative to Control | | | | |
|---|---|---|---|---|---|---|
| Amino Acid | Conc. | | Std. | Number | P- | |
| Supplement | (X) | Mean | Deviation | of values | value* | Significance |
| LIVRQNAC | 40 | 0.86 | 0.04 | 4 | ** | 0.0056 |
| LIVRQNAC | 30 | 0.82 | 0.04 | 4 | *** | 0.0005 |
| LIVRQNAC | 20 | 0.84 | 0.02 | 4 | ** | 0.0011 |
| LIVRQNAC | 10 | 0.80 | 0.04 | 4 | *** | 0.0002 |
| LIVRQNAC | 1 | 1.00 | 0.09 | 4 | | |
| LIVRQNAC + G | 40 | 0.72 | 0.08 | 4 | **** | 0.0001 |
| LIVRQNAC + G | 30 | 0.75 | 0.04 | 4 | **** | 0.0001 |
| LIVRQNAC + G | 20 | 0.75 | 0.04 | 4 | **** | 0.0001 |
| LIVRQNAC + G | 10 | 0.78 | 0.05 | 4 | **** | 0.0001 |
| LIVRQNAC + G | 1 | 1.00 | 0.03 | 4 | | |
| LIVRQNAC + S | 40 | 0.77 | 0.03 | 4 | **** | 0.0001 |
| LIVRQNAC + S | 30 | 0.79 | 0.06 | 4 | *** | 0.0002 |
| LIVRQNAC + S | 20 | 0.82 | 0.06 | 4 | *** | 0.001 |
| LIVRQNAC + S | 10 | 0.81 | 0.06 | 4 | *** | 0.0005 |
| LIVRQNAC + S | 1 | 1.00 | 0.06 | 4 | | |
| LIV | 40 | 0.89 | 0.07 | 4 | ns | 0.2215 |
| LIV | 30 | 1.00 | 0.12 | 4 | ns | 0.9999 |
| LIV | 20 | 0.96 | 0.07 | 4 | ns | 0.8327 |
| LIV | 10 | 0.98 | 0.04 | 4 | ns | 0.9724 |
| LIV | 1 | 1.00 | 0.09 | 4 | | |
| LIVRQ | 40 | 0.60 | 0.03 | 4 | **** | 0.0001 |
| LIVRQ | 30 | 0.61 | 0.09 | 4 | **** | 0.0001 |
| LIVRQ | 20 | 0.66 | 0.03 | 4 | **** | 0.0001 |
| LIVRQ | 10 | 0.73 | 0.05 | 4 | *** | 0.0004 |
| LIVRQ | 1 | 1.00 | 0.13 | 4 | | |
| RQNAC | 40 | 0.47 | 0.07 | 4 | **** | 0.0001 |
| RQNAC | 30 | 0.50 | 0.02 | 4 | **** | 0.0001 |
| RQNAC | 20 | 0.60 | 0.05 | 4 | **** | 0.0001 |
| RQNAC | 10 | 0.66 | 0.04 | 4 | **** | 0.0001 |
| RQNAC | 1 | 1.00 | 0.06 | 4 | | |
| N-Acetyl Cysteine | 40 | 0.88 | 0.02 | 4 | * | 0.0217 |
| N-Acetyl Cysteine | 20 | 1.09 | 0.05 | 4 | ns | 0.1033 |
| N-Acetyl Cysteine | 10 | 1.11 | 0.10 | 4 | * | 0.043 |
| N-Acetyl Cysteine | 5 | 1.03 | 0.02 | 4 | ns | 0.9209 |
| N-Acetyl Cysteine | 0 | 1.00 | 0.06 | 4 | | |

TABLE 36
Fold change of Hsp47 gene expression after administration of a single amino acid composition,
normalized to Gapdh expression in the first donor

| | | Hsp47 Fold Expression Relative to Control | | | | |
|---|---|---|---|---|---|---|
| Amino Acid | Conc. | | Std. | Number | P- | |
| Supplement | (μM) | Mean | Deviation | of values | value* | Significance |
| Valine | 23420 | 1.00 | 0.04 | 4 | ns | 0.9995 |
| Valine | 11710 | 1.05 | 0.08 | 4 | ns | 0.5447 |
| Valine | 4684 | 1.05 | 0.06 | 4 | ns | 0.5443 |
| Valine | 234 | 1.00 | 0.06 | 4 | | |
| Arginine | 5440 | 1.25 | 0.08 | 4 | *** | 0.0001 |
| Arginine | 2720 | 1.05 | 0.05 | 4 | ns | 0.5692 |
| Arginine | 1088 | 0.94 | 0.02 | 4 | ns | 0.3705 |
| Arginine | 109 | 1.00 | 0.06 | 4 | | |
| Glutamine | 22484 | 0.51 | 0.05 | 4 | **** | 0.0001 |
| Glutamine | 11242 | 0.62 | 0.05 | 4 | **** | 0.0001 |
| Glutamine | 3747 | 0.82 | 0.08 | 4 | * | 0.0193 |
| Glutamine | 749 | 1.17 | 0.06 | 4 | * | 0.0341 |
| Glutamine | 562 | 1.00 | 0.13 | 4 | | |
| Isoleucine | 6639 | 0.99 | 0.11 | 4 | ns | 0.9995 |
| Isoleucine | 3320 | 1.22 | 0.32 | 4 | ns | 0.3335 |
| Isoleucine | 1328 | 1.07 | 0.15 | 4 | ns | 0.9366 |
| Isoleucine | 66 | 1.00 | 0.14 | 4 | | |
| Leucine | 15270 | 0.94 | 0.08 | 4 | ns | 0.7669 |
| Leucine | 7635 | 1.13 | 0.12 | 4 | ns | 0.2585 |
| Leucine | 3054 | 1.07 | 0.13 | 4 | ns | 0.6982 |
| Leucine | 153 | 1.00 | 0.07 | 4 | | |
| N-Acetyl Cysteine | 10000 | 0.88 | 0.02 | 4 | * | 0.0217 |
| N-Acetyl Cysteine | 5000 | 1.09 | 0.05 | 4 | ns | 0.1033 |
| N-Acetyl Cysteine | 2500 | 1.11 | 0.10 | 4 | * | 0.043 |
| N-Acetyl Cysteine | 1000 | 1.03 | 0.02 | 4 | ns | 0.9209 |
| N-Acetyl Cysteine | 0 | 1.00 | 0.06 | 4 | | |

TABLE 37
Fold change of Hsp47 gene expression after administration of an amino acid composition,
normalized to Gapdh expression in second donor.

| | | Hsp47 Fold Expression Relative to Control | | | | |
|---|---|---|---|---|---|---|
| Amino Acid | Conc. | | Std. | Number | P- | |
| Supplement | (X) | Mean | Deviation | of values | value* | Significance |
| LIVRQNAC | 40 | 0.75 | 0.03 | 4 | **** | 0.0001 |
| LIVRQNAC | 30 | 0.77 | 0.05 | 4 | **** | 0.0001 |
| LIVRQNAC | 20 | 0.80 | 0.03 | 4 | *** | 0.0001 |
| LIVRQNAC | 10 | 0.78 | 0.06 | 4 | **** | 0.0001 |
| LIVRQNAC | 1 | 1.00 | 0.05 | 4 | | |
| LIVRQNAC + G | 40 | 0.64 | 0.07 | 4 | **** | 0.0001 |
| LIVRQNAC + G | 30 | 0.70 | 0.02 | 4 | *** | 0.0002 |
| LIVRQNAC + G | 20 | 0.72 | 0.06 | 4 | *** | 0.0005 |
| LIVRQNAC + G | 10 | 0.70 | 0.12 | 4 | *** | 0.0002 |
| LIVRQNAC + G | 1 | 1.00 | 0.08 | 4 | | |
| LIVRQNAC + S | 40 | 0.77 | 0.05 | 4 | ** | 0.0015 |
| LIVRQNAC + S | 30 | 0.79 | 0.06 | 4 | ** | 0.0029 |
| LIVRQNAC + S | 20 | 0.84 | 0.07 | 4 | * | 0.0225 |
| LIVRQNAC + S | 10 | 0.82 | 0.09 | 4 | * | 0.0115 |
| LIVRQNAC + S | 1 | 1.00 | 0.09 | 4 | | |
| LIV | 40 | 1.14 | 0.16 | 4 | ns | 0.197 |
| LIV | 30 | 1.04 | 0.05 | 4 | ns | 0.9267 |
| LIV | 20 | 1.04 | 0.05 | 4 | ns | 0.9561 |
| LIV | 10 | 0.98 | 0.10 | 4 | ns | 0.9961 |
| LIV | 1 | 1.00 | 0.08 | 4 | | |
| LIVRQ | 40 | 1.03 | 0.10 | 4 | ns | 0.9929 |
| LIVRQ | 30 | 1.00 | 0.11 | 4 | ns | 0.9999 |
| LIVRQ | 20 | 0.97 | 0.13 | 4 | ns | 0.9869 |
| LIVRQ | 10 | 1.05 | 0.12 | 4 | ns | 0.9461 |
| LIVRQ | 1 | 1.00 | 0.09 | 4 | | |
| RQNAC | 40 | 0.70 | 0.06 | 3 | **** | 0.0001 |
| RQNAC | 30 | 0.64 | 0.06 | 4 | **** | 0.0001 |
| RQNAC | 20 | 0.72 | 0.04 | 4 | **** | 0.0001 |
| RQNAC | 10 | 0.76 | 0.04 | 4 | *** | 0.0002 |
| RQNAC | 1 | 1.00 | 0.09 | 4 | | |
| N-Acetyl Cysteine | 40 | 0.68 | 0.05 | 4 | **** | 0.0001 |
| N-Acetyl Cysteine | 20 | 0.79 | 0.06 | 4 | *** | 0.0006 |
| N-Acetyl Cysteine | 10 | 0.78 | 0.07 | 4 | *** | 0.0003 |
| N-Acetyl Cysteine | 5 | 0.78 | 0.06 | 4 | *** | 0.0004 |
| N-Acetyl Cysteine | 0 | 1.00 | 0.04 | 4 | | |

TABLE 38
Fold change of Hsp47 gene expression after administration of a single amino acid composition,
normalized to Gapdh expression in second donor.

| | | Hsp47 Fold Expression Relative to Control | | | | |
|---|---|---|---|---|---|---|
| Amino Acid | Conc. | | Std. | Number | P- | |
| Supplement | (μM) | Mean | Deviation | of values | value* | Significance |
| Valine | 23420 | 0.89 | 0.12 | 4 | ns | 0.395 |
| Valine | 11710 | 0.99 | 0.14 | 4 | ns | 0.9958 |
| Valine | 4684 | 1.02 | 0.11 | 4 | ns | 0.9918 |
| Valine | 234 | 1.00 | 0.09 | 4 | | |
| Arginine | 5440 | 1.25 | 0.10 | 4 | ** | 0.0012 |
| Arginine | 2720 | 1.15 | 0.05 | 4 | * | 0.0365 |
| Arginine | 1088 | 1.04 | 0.09 | 4 | ns | 0.835 |
| Arginine | 109 | 1.00 | 0.03 | 4 | | |
| Glutamine | 22484 | 0.87 | 0.13 | 4 | ns | 0.1668 |
| Glutamine | 11242 | 0.98 | 0.07 | 4 | ns | 0.983 |
| Glutamine | 3747 | 1.09 | 0.08 | 4 | ns | 0.4276 |
| Glutamine | 749 | 1.20 | 0.09 | 4 | * | 0.0211 |
| Glutamine | 562 | 1.00 | 0.02 | 4 | | |
| Isoleucine | 6639 | 0.94 | 0.02 | 4 | ns | 0.396 |
| Isoleucine | 3320 | 0.92 | 0.03 | 4 | ns | 0.2348 |
| Isoleucine | 1328 | 0.90 | 0.04 | 4 | ns | 0.109 |
| Isoleucine | 66 | 1.00 | 0.12 | 4 | | |
| Leucine | 15270 | 1.04 | 0.06 | 4 | ns | 0.6052 |
| Leucine | 7635 | 1.03 | 0.07 | 4 | ns | 0.7746 |
| Leucine | 3054 | 1.00 | 0.04 | 4 | ns | 0.9999 |
| Leucine | 153 | 1.00 | 0.05 | 4 | | |
| N-Acetyl Cysteine | 10000 | 0.68 | 0.05 | 4 | **** | 0.0001 |
| N-Acetyl Cysteine | 5000 | 0.79 | 0.06 | 4 | *** | 0.0006 |
| N-Acetyl Cysteine | 2500 | 0.78 | 0.07 | 4 | *** | 0.0003 |
| N-Acetyl Cysteine | 1000 | 0.78 | 0.06 | 4 | *** | 0.0004 |
| N-Acetyl Cysteine | 0 | 1.00 | 0.04 | 4 | | |

TABLE 39
Fold change of Hsp47 gene expression after administration of an amino acid composition,
normalized to Gapdh expression in third donor

| | | Hsp47 Fold Expression Relative to Control | | | | |
|---|---|---|---|---|---|---|
| Amino Acid | Conc. | | Std. | Number | P- | |
| Supplement | (X) | Mean | Deviation | of values | value* | Significance |
| LIVRQNAC | 40 | 0.80 | 0.08 | 4 | ** | 0.0042 |
| LIVRQNAC | 30 | 0.82 | 0.07 | 4 | * | 0.0114 |
| LIVRQNAC | 20 | 0.83 | 0.06 | 4 | * | 0.0174 |
| LIVRQNAC | 10 | 0.85 | 0.05 | 4 | * | 0.0358 |
| LIVRQNAC | 1 | 1.00 | 0.06 | 3 | | |
| LIVRQNAC + G | 40 | 0.68 | 0.09 | 4 | *** | 0.0003 |
| LIVRQNAC + G | 30 | 0.70 | 0.04 | 4 | *** | 0.0005 |
| LIVRQNAC + G | 20 | 0.78 | 0.11 | 4 | ** | 0.007 |
| LIVRQNAC + G | 10 | 0.76 | 0.05 | 4 | ** | 0.0039 |
| LIVRQNAC + G | 1 | 1.00 | 0.11 | 4 | | |
| LIVRQNAC + S | 40 | 0.75 | 0.09 | 2 | * | 0.0106 |
| LIVRQNAC + S | 30 | 0.74 | 0.05 | 4 | ** | 0.0018 |
| LIVRQNAC + S | 20 | 0.78 | 0.07 | 4 | ** | 0.0058 |
| LIVRQNAC + S | 10 | 0.79 | 0.05 | 4 | ** | 0.0076 |
| LIVRQNAC + S | 1 | 1.00 | 0.13 | 4 | | |
| LIV | 40 | 0.96 | 0.09 | 4 | ns | 0.8499 |
| LIV | 30 | 0.99 | 0.08 | 4 | ns | 0.9974 |
| LIV | 20 | 1.04 | 0.06 | 4 | ns | 0.8348 |
| LIV | 10 | 1.01 | 0.04 | 4 | ns | 0.9976 |
| LIV | 1 | 1.00 | 0.08 | 4 | | |
| LIVRQ | 40 | 0.99 | 0.01 | 4 | ns | 0.9993 |
| LIVRQ | 30 | 1.05 | 0.02 | 4 | ns | 0.6878 |
| LIVRQ | 20 | 1.06 | 0.10 | 4 | ns | 0.5142 |
| LIVRQ | 10 | 1.07 | 0.09 | 4 | ns | 0.3719 |
| LIVRQ | 1 | 1.00 | 0.06 | 4 | | |
| RQNAC | 40 | 0.72 | 0.08 | 3 | *** | 0.0001 |
| RQNAC | 30 | 0.75 | 0.03 | 4 | *** | 0.0001 |
| RQNAC | 20 | 0.81 | 0.03 | 4 | ** | 0.0018 |
| RQNAC | 10 | 0.95 | 0.06 | 4 | ns | 0.5648 |
| RQNAC | 1 | 1.00 | 0.09 | 4 | | |
| N-Acetyl Cysteine | 40 | 0.60 | 0.04 | 4 | **** | 0.0001 |
| N-Acetyl Cysteine | 20 | 0.71 | 0.09 | 4 | ** | 0.0015 |
| N-Acetyl Cysteine | 10 | 0.79 | 0.08 | 4 | * | 0.0167 |
| N-Acetyl Cysteine | 5 | 0.88 | 0.09 | 4 | ns | 0.2154 |
| N-Acetyl Cysteine | 0 | 1.00 | 0.13 | 4 | | |

TABLE 40
Fold change of Hsp47 gene expression after administration of a single amino acid composition,
normalized to Gapdh expression in second donor.

| | | Hsp47 Fold Expression Relative to Control | | | | |
|---|---|---|---|---|---|---|
| Amino Acid | Conc. | | Std. | Number | P- | |
| Supplement | (μM) | Mean | Deviation | of values | value* | Significance |
| Valine | 23420 | 0.95 | 0.11 | 4 | ns | 0.8891 |
| Valine | 11710 | 0.95 | 0.17 | 4 | ns | 0.909 |
| Valine | 4684 | 0.97 | 0.12 | 4 | ns | 0.9786 |
| Valine | 234 | 1.00 | 0.07 | 4 | | |
| Arginine | 5440 | 1.21 | 0.14 | 4 | ns | 0.068 |
| Arginine | 2720 | 1.11 | 0.13 | 4 | ns | 0.4558 |
| Arginine | 1088 | 1.03 | 0.11 | 4 | ns | 0.9749 |
| Arginine | 109 | 1.00 | 0.08 | 4 | | |
| Glutamine | 22484 | 0.99 | 0.13 | 3 | ns | 0.9991 |
| Glutamine | 11242 | 0.97 | 0.09 | 4 | ns | 0.9571 |
| Glutamine | 3747 | 1.03 | 0.09 | 4 | ns | 0.9544 |
| Glutamine | 749 | 1.02 | 0.09 | 4 | ns | 0.9949 |
| Glutamine | 562 | 1.00 | 0.04 | 4 | | |
| Isoleucine | 6639 | 1.06 | 0.08 | 4 | ns | 0.7622 |
| Isoleucine | 3320 | 0.98 | 0.02 | 4 | ns | 0.9632 |
| Isoleucine | 1328 | 1.00 | 0.12 | 4 | ns | 0.999 |
| Isoleucine | 66 | 1.00 | 0.11 | 4 | | |
| Leucine | 15270 | 1.10 | 0.05 | 4 | ns | 0.4295 |
| Leucine | 7635 | 1.04 | 0.09 | 4 | ns | 0.9117 |
| Leucine | 3054 | 1.06 | 0.15 | 4 | ns | 0.7532 |
| Leucine | 153 | 1.00 | 0.07 | 4 | | |
| N-Acetyl Cysteine | 10000 | 0.60 | 0.04 | 4 | **** | 0.0001 |
| N-Acetyl Cysteine | 5000 | 0.71 | 0.09 | 4 | ** | 0.0015 |
| N-Acetyl Cysteine | 2500 | 0.79 | 0.08 | 4 | * | 0.0167 |
| N-Acetyl Cysteine | 1000 | 0.88 | 0.09 | 4 | ns | 0.2154 |
| N-Acetyl Cysteine | 0 | 1.00 | 0.13 | 4 | | |

### Example 9. Triculture Model for Recapitulating the Liver Microenvironment for Interrogating Fibrosis

Cell Seeding and Maintenance

Triculture model including the three major cell types of the liver (hepatocytes, hepatic macrophages and stellate cells) was developed to assess the effect of the amino acids combination L-leucine, L-isoleucine, L-valine, L-arginine, L-glutamine, and N-acetylcysteine (LIVRQNAC) on fibrosis.

A 96-well or 12-well transwell (corning) was used to co-culture hepatocytes, macrophages, and stellate cells isolated from healthy donors.

Primary human hepatic stellate cells obtained from Samsara Sciences and grown in Complete HSC Medium to −80% confluence in T150 flasks were seeded on the undersurface of the membrane of transwells previously coated with collagen (Corning).

Once the stellate cells were seeded, primary human PBMC derived macrophages were also added on the undersurface of the membrane. In the Transwell, both cells were plated in the hepatocytes plating media (William's E medium (Gibco) supplemented with 10% heat-inactivated FBS (Atlanta Bio), 2 mM Glutamax (Gibco), and 0.2% Primocin (InVivoGen) and incubated for 6 hours at 37° C., 5% CO2.

After 6 hours of incubation, primary hepatocytes from a healthy human donor were seeded on the collagen gel on the top surface of the transwell. The triculture was incubated at 37° C., 5% CO2 in hepatocyte plating media described above. After 6 hours, cells were washed once and incubated overnight at 37° C., 5% CO2 in hepatocytes plating media. On day 1, cells were washed once and incubated in hepatocytes defined medium (Corning) supplemented with 2 mM Glutamax (Gibco), and 1× Penicillin/Streptomycin (P/S) overnight at 37° C., 5% CO2.

### Amino Acids Pre-Treatment

On day 2, cells were washed twice with DPBS 1× (Gibco) and maintained in:
a. Amino acid-free WEM (US Biologicals) supplemented with 11 mM Glucose (Sigma), 0.272 mM Sodium Pyruvate (Sigma), 1× P/S (Gibco) and containing a defined custom amino acid concentration based on the mean physiological concentrations in blood; or
b. The same media described above with one concentration of defined amino acid compositions LIVRQNAC at 30× or 40×.

Cells were maintained in the defined media (a. and b.) for 24 hours at 37° C., 5% CO2.

Co-Treatment with Free Fatty Acids and Different Amino Acids Combination

After 24 h pre-treatment, cells were maintained in the same media described above and exposed to free fatty acids (FFAs) at 250 uM with a ratio of 2:1 (Oleate:Palmitate) supplemented with TNF-α (Thermofisher) at 1 ng/ml±LIVRQNAC. After 24 hours of incubation at 37° C., 5% CO2, media was removed from each side of the transwell separately and cells were incubated in the same conditions described above for an additional 48 hours.

Cytokine/Chemokine and Procollagen Iα1 Analysis after 24 h by ELISA

Supernatants from both sides of the 96-well transwell plate were used to analyze a multiplex panel of analytes: IL6, IL8, MCP1, IP10, Gro alpha, and Procollagen Iα1 (fireplex kit, Abcam). YKL40 was measured from the supernatant collected from the 12-well transwell plate by ELISA (Human Chitinase 3-like 1 (YKL40) Quantikine ELISA, R&D systems).

### Procollagen Iα1 Secretion

Table 41 shows the fold change in procollagen Iα1 secreted by the stellate cells treated with (FFAs TNFα)+LIVRQNAC at 30× normalized to the FFAs+TNFα baseline. Statistical significance calculated by T-Test shows that LIVRQNAC significantly decreased procollagen Iα1 secretion. Procollagen Iα1 level from the hepatocytes side was measured and showed no difference between both treatments (table 42).

TABLE 41
Fold change of procollagen Iα1 secretion by stellate cells in triculture after administration
of LIVRQNAC at 30× compared to LIVRQNAC at 1×

| | | Procollagen Iα1 Secretion (Fold Change of 1×) | | | | |
|---|---|---|---|---|---|---|
| Amino Acid | Conc. | Mean | Std. | Number | P- | |
| Supplement | (X) | (log2) | Deviation | of values | value* | Significance |
| LIVRQNAC | 30 | −1.2E+00 | 0.00 | 3 | 0.004 | * |
| LIVRQNAC | 1 | −4.7E−02 | 0.44 | 3 | n/a | n/a |

TABLE 42
Fold change of procollagen Iα1 level measured from the hepatocytes sides in triculture after
administration of LIVRQNAC at 30× compared to LIVRQNAC at 1×

| | | Procollagen Iα1 Secretion (Fold Change of 1×) | | | | |
|---|---|---|---|---|---|---|
| Amino Acid | Conc. | Mean | Std. | Number | P- | |
| Supplement | (X) | (log2) | Deviation | of values | value* | Significance |
| LIVRQNAC | 30 | 3.4E−02 | 0.002 | 3 | 0.1509 | ns |
| LIVRQNAC | 1 | −7.5E−05 | 0.02 | 3 | n/a | n/a |

Tables 43 and 44 show the fold change in cytokines and chemokines secreted by either macrophages and the stellate cells or Hepatocytes side respectively treated with FFAs+TNFα+LIVRQNAC at 30× normalized to the FFAs+TNFα baseline (LIVRQNAC at 1×). Several proinflammatory cytokines (IL-6, IL-8, IP-10, and GROalpha (CXCL1)) and chemokine (MCP1) which have established chemoattractant properties and shown to be upregulated in NASH patients were measured. Statistical significance calculated by T-Test shows that treatment with LIVRQNAC at 30× significantly decreased IL-6, IP-10, GROalpha (CXCL1), and MCP1 levels as compared to the control LIVRQNAC at 1×. IL-8 level was also reduced when treated with LIVRQNAC 30×, however did not show statistical significance compared to LIVRQNAC 1×.

TABLE 43
Fold change of Cytokines and Chemokines secretion by macrophages and stellate
cells after administration of LIVRQNAC at 30× compared to LIVRQNAC at 1×

| Cytokines/ | Amino Acids | Conc. | Mean | Std. | Number | P- | |
|---|---|---|---|---|---|---|---|
| Chemokines | Supplement | (X) | (log2) | Deviation | of values | value* | Significance |
| IL-6 | LIVRQNAC | 30 | −2.9E+00 | 0.16 | 3 | 0.0015 | ** |
| IL-6 | LIVRQNAC | 1 | −6.3E−05 | 0.02 | 3 | n/a | n/a |
| IL-8 | LIVRQNAC | 30 | −8.2E−01 | 0.52 | 3 | 0.1409 | ns |
| IL-8 | LIVRQNAC | 1 | −1.0E−02 | 0.24 | 3 | n/a | n/a |
| IP-10 | LIVRQNAC | 30 | −1.4E+00 | 0.31 | 3 | 0.0069 | ** |
| IP-10 | LIVRQNAC | 1 | −2.4E−02 | 0.33 | 3 | n/a | n/a |
| GROalpha | LIVRQNAC | 30 | −9.4E−01 | 0.48 | 3 | 0.0785 | ns |
| (CXCL1) | | | | | | | |
| GROalpha | LIVRQNAC | 1 | −2.2E−04 | 0.04 | 3 | n/a | n/a |
| (CXCL1 | | | | | | | |
| MCP1 | LIVRQNAC | 30 | −1.4E+00 | 0.27 | 3 | 0.0061 | ** |
| MCP1 | LIVRQNAC | 1 | −1.8E−04 | 0.03 | 3 | n/a | n/a |

TABLE 44
Fold change of Cytokines and Chemokines secretion by Hepatocytes cells after
administration of LIVRQNAC at 30× compared to LIVRQNAC at 1×

| Cytokines/ | Amino Acids | Conc. | Mean | Std. | Number | P- | |
|---|---|---|---|---|---|---|---|
| Chemokines | Supplement | (X) | (log2) | Deviation | of values | value* | Significance |
| IL-6 | LIVRQNAC | 30 | −2.7E+00 | 0.58 | 3 | 0.0018 | ** |
| IL-6 | LIVRQNAC | 1 | −1.1E−02 | 0.23 | 3 | n/a | n/a |
| IL-8 | LIVRQNAC | 30 | −1.4E+00 | 0.29 | 3 | 0.0541 | ns |
| IL-8 | LIVRQNAC | 1 | 0.0E+00 | 0.00 | 3 | n/a | n/a |
| IP-10 | LIVRQNAC | 30 | −2.7E+00 | 0.26 | 3 | 0.0001 | **** |
| IP-10 | LIVRQNAC | 1 | −9.3E−04 | 0.06 | 3 | n/a | n/a |
| GROalpha | LIVRQNAC | 30 | −1.8E+00 | 0.04 | 3 | 0.0002 | *** |
| (CXCL1) | | | | | | | |
| GROalpha | LIVRQNAC | 1 | −2.6E−03 | 0.11 | 3 | n/a | n/a |
| (CXCL1) | | | | | | | |
| MCP1 | LIVRQNAC | 30 | −1.6E+00 | 0.03 | 3 | 0.0039 | ** |
| MCP1 | LIVRQNAC | 1 | −1.6E−02 | 0.27 | 3 | n/a | n/a |

Tables 45 and 46 show the fold change in YKL-40 secreted by either macrophages and the stellate cells or Hepatocytes treated with FFAs TNFα+LIVRQNAC at 40× normalized to the LIVRQNAC 1×.

Plasma levels of YKL40 (also called chitinase-3-like protein 1 [CHI3L1]) are increased in several inflammatory diseases, including NASH. It has been shown that YKL40 plasma levels increased in NAFLD patients with the progression of fibrosis. Statistical significance calculated by T-Test shows that LIVRQNAC at 40× decreases hepatocytes YKL40 level significantly. YKL-40 level measured from the macrophages and stellate cells side was also reduced when treated with LIVRQNAC 40× but didn't show statistical significance compared to LIVRQNAC 1× treatment.

TABLE 45
Fold change of YKL40 secretion by stellate cells and macrophages after administration
of LIVRQNAC at 40× compared to LIVRQNAC at 1×

| | | YKL40 secretion (Fold change of 1×) | | | | |
|---|---|---|---|---|---|---|
| Amino Acid | Conc. | | Std. | Number | P- | |
| Supplement | (X) | Mean | Deviation | of values | value* | Significance |
| LIVRQNAC | 30 | −6.0E−01 | 0.28 | 2 | 0.148 | ns |
| LIVRQNAC | 1 | −9.2E−03 | 0.23 | 2 | n/a | n/a |

TABLE 46
Fold change of YKL40 secretion by Hepatocytes after administration of
LIVRQNAC at 40× compared to LIVRQNAC at 1×

| | | YKL40 secretion (Fold change of 1×) | | | | |
|---|---|---|---|---|---|---|
| Amino Acid | Conc. | | Std. | Number | P- | |
| Supplement | (X) | Mean | Deviation | of values | value* | Significance |
| LIVRQNAC | 30 | −1.5E+00 | 0.13 | 2 | 0.0097 | ** |
| LIVRQNAC | 1 | −4.9E−03 | 0.17 | 2 | n/a | n/a |

### Example 10: TGFβ1 Induced Proliferation of Hepatic Stellate Cells

Proliferation of hepatic stellate cells is a key phenotypic feature of activated hepatic stellate cells. Primary human hepatic stellate cells were obtained from Samsara Sciences based on the following criteria for selecting donors: adult age (between 18 and 50 years), normal BMI (>18.5 and <25), and absence of confounding liver disease. Cells from three different donors were grown in Complete HSC Medium to −80% confluence in T75 or T150 flasks below passage 10 were seeded into sterile, collagen I coated, 96-well optical plastic microplates (ThermoScientific, 152036) at 6000 cells per well (˜1250 cells per cm²) and incubated overnight at 37° C., 5% CO2 in a humidified incubator in DMEM with 2% Fetal Bovine Serum and 1% Antibiotic-Antimycotic.

After the overnight incubation, plates were removed from the incubator and the medium was gently pipetted off and washed twice with 150 μL per well DPBS. The DPBS was removed and the pretreatment medium (1×HMDB amino acid DMEM+1% Antibiotic-Antimycotic, 10 mM HEPES, ±supplemental treatment dose at a multiple (×) of the HMDB amino acid concentration) was applied to the cells at 150 μL per well. Each treatment and dose was tested in triplicate wells per plate. Vehicle control was tested in 6 replicate wells per plate. Plates were incubated overnight. After overnight pretreatment, the medium was removed from the cells and the same pretreatment medium, supplemented with 3 ng/mL TGFβ1, was applied. To assess proliferation, cells were labeled with 10 μM EdU (5-ethynyl-2′-deoxyuridine) which is incorporated into DNA during active DNA synthesis. Plates were then incubated for 24 hours at 37° C., 5% CO₂.

After 24-hour stimulus, supernatant was removed and frozen at −80° C. in two separate aliquots. Cells were then washed with DPBS and fixed with 4% paraformaldehyde solution for 20 minutes. Cells were permeabilized with 0.1% Triton X-100 and EdU was labeled using the Click-iT™ EdU Alexa Fluor™ 555 HCS Assay (Invitrogen) according to the manufacturer's instructions. Nuclei were labeled with Hoechst 33342, a cell permeable DNA binding dye.

Cells were imaged using an ImageXpress Micro Confocal high content imager (Molecular Devices) using a 10× Plan Apo objective. Twelve frames were imaged per well. EdU labeled with Alexa Fluor™ 555 was detected in the Texas Red channel. Nuclei labeled with Hoechst 33342 were detected in the DAPI channel. Image analysis was performed using MetaXpress Version 6.2.3.733 (Molecular Devices). The number of proliferating cells, defined as those nuclei that were positive for EdU labeling (EdU+) and the total nuclei count were determined for each condition. The percentage EdU positive cells (% EdU+) was determined as the number of EdU positive nuclei divided by the total number of nuclei for each well. Fold change in nuclei count and % EdU+ cells were calculated relative to the baseline amino acid (1×HMDB) vehicle (PBS) condition stimulated with 3 ng/mL TGFβ1. The mean of each phenotype's measurement in 3 ng/mL TGFβ1 treated PBS vehicle wells is defined as the baseline. The phenotype measurement in each well is divided by this baseline. A score that equals 1 means no change from baseline. A score less or more than 1 means decrease or increase, respectively. Statistical analysis (mean, standard deviation calculation and two-tailed t-test) is done on the log 2 transformed scores.

### Results

Table 47 shows the log 2 transform of fold change in the percentage of actively proliferating EdU positive cells, relative to the PBS vehicle condition in primary human hepatic stellate cells from three different donors. LIVRQNAC reduced the percentage of actively proliferating EdU positive cells in all three donors relative to 3 ng/mL TGFβ1 vehicle. Table 48 shows the log 2 transform of fold change in nuclei count relative to the PBS vehicle condition in primary human hepatic stellate cells from three different donors. LIVRQNAC reduced nuclei count at the highest two dose conditions in two out of the three donors tested relative to 3 ng/mL TGFβ1 vehicle.

TABLE 47

| Experiment | Treatment | Concentration | Average | Std Dev. | Number | Significance | p-value |
|---|---|---|---|---|---|---|---|
| Donor 1 | PBS | 1 | −0.039 | 0.347 | 24 | 0 | 1 |
| Donor 1 | LIVRQNAC | 10 | −1.180 | 0.464 | 3 | 1 | 0.0140 |
| Donor 1 | LIVRQNAC | 20 | −1.767 | 0.258 | 3 | 1 | 0.0009 |
| Donor 1 | LIVRQNAC | 40 | −2.675 | 0.237 | 3 | 1 | 0.0001 |
| Donor 2 | PBS | 1 | −0.005 | 0.122 | 24 | 0 | 1.0000 |
| Donor 2 | LIVRQNAC | 10 | −0.813 | 0.195 | 3 | 1 | 0.0005 |
| Donor 2 | LIVRQNAC | 20 | −1.235 | 0.422 | 3 | 1 | 0.0004 |
| Donor 2 | LIVRQNAC | 40 | −3.540 | 0.365 | 3 | 1 | 2.00E−07 |
| Donor 3 | PBS | 1 | −0.012 | 0.188 | 24 | 0 | 1.0000 |
| Donor 3 | LIVRQNAC | 10 | −0.653 | 0.081 | 3 | 1 | 3.51E−05 |
| Donor 3 | LIVRQNAC | 20 | −1.355 | 0.089 | 3 | 1 | 2.83E−07 |
| Donor 3 | LIVRQNAC | 40 | −3.431 | 0.529 | 3 | 1 | 7.83E−07 |

TABLE 48

| Experiment | Treatment | Concentration | Average | Std Dev. | Number | Significance | p-value |
|---|---|---|---|---|---|---|---|
| Donor 1 | PBS | 1 | −0.008 | 0.152 | 24 | 0 | 1 |
| Donor 1 | LIVRQNAC | 10 | 0.19 | 0.095 | 3 | 0 | 0.1485574 |
| Donor 1 | LIVRQNAC | 20 | 0.101 | 0.081 | 3 | 0 | 0.3896755 |
| Donor 1 | LIVRQNAC | 40 | −0.058 | 0.111 | 3 | 0 | 0.7172079 |
| Donor 2 | PBS | 1 | −0.001 | 0.06 | 24 | 0 | 1 |
| Donor 2 | LIVRQNAC | 10 | −0.083 | 0.209 | 3 | 0 | 0.3375736 |
| Donor 2 | LIVRQNAC | 20 | −0.242 | 0.183 | 3 | 1 | 0.0110886 |
| Donor 2 | LIVRQNAC | 40 | −1.358 | 0.221 | 3 | 1 | 8.93E−07 |
| Donor 3 | PBS | 1 | −0.001 | 0.059 | 24 | 0 | 1 |
| Donor 3 | LIVRQNAC | 10 | 0.076 | 0.074 | 3 | 0 | 0.1738019 |
| Donor 3 | LIVRQNAC | 20 | −0.161 | 0.105 | 3 | 1 | 0.0291969 |
| Donor 3 | LIVRQNAC | 40 | −1.255 | 0.258 | 3 | 1 | 7.20E−06 |

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A method for reducing fibrosis in a subject, comprising administering to the subject in need thereof an effective amount of a composition comprising:
a) a leucine amino acid entity,
b) an arginine amino acid entity,
c) glutamine amino acid entity; and
d) a N-acetylcysteine (NAC)-entity;
provided that:
the fibrosis is not liver fibrosis,
thereby reducing the fibrosis in the subject.

2. A method of treating a fibrotic condition or disorder in a subject in need thereof, comprising administering to the subject an effective amount of a composition comprising:
a) a leucine-amino acid entity
b) an arginine-amino acid entity,
c) glutamine-amino acid entity; and
d) NAC-entity;
provided that:
the fibrotic condition or disorder is not a liver fibrotic condition or disorder,
thereby treating the fibrotic condition or disorder.

3. A composition for use in reducing fibrosis in a subject, comprising an effective amount of a composition comprising:
a) a leucine amino acid entity,
b) an arginine amino acid entity,
c) glutamine amino acid entity; and
d) a N-acetylcysteine (NAC)-entity;
provided that:
the fibrosis is not liver fibrosis.

4. A composition for use in intreating a fibrotic condition or disorder in a subject in need thereof, comprising an effective amount of a composition comprising:
a) a leucine-amino acid entity
b) an arginine-amino acid entity,
c) glutamine-amino acid entity; and
d) NAC-entity;
provided that:
the fibrotic condition or disorder is not a liver fibrotic condition or disorder.

5. The method of claim 1 or 2, or composition for use of claim 3 or 4, wherein the fibrotic condition or disorder is chosen from a lung fibrotic condition or disorder, a heart or vasculature fibrotic condition or disorder, a kidney fibrotic condition or disorder, a pancreas fibrotic condition or disorder, a skin fibrotic condition or disorder, a gastrointestinal fibrotic condition or disorder, a bone marrow or hematopoietic tissue fibrotic condition or disorder, a nervous system fibrotic condition or disorder, an eye fibrotic condition or disorder, or a combination thereof.

6. The method of any of claim 1, 2, or 5, or composition for use of any of claims 3-5, wherein administration of the composition results in a reduction or inhibition of one, two, three, four, five, six, or more (e.g., all) of:
(a) formation or deposition of tissue fibrosis;
(b) the size, cellularity, composition, or cellular content, of a fibrotic lesion;
(c) the collagen of a fibrotic lesion;
(d) the collagen or hydroxyproline content, of a fibrotic lesion;
(e) expression or activity of a fibrogenic protein;
(f) fibrosis associated with an inflammatory response; or
(g) weight loss associated with fibrosis.

7. The method of any of claim 1, 2, 5, or 6, or composition for use of any of claims 3-6, wherein the method further comprises determining the level of one, two, three, four, five, six, seven, eight, nine, ten, 11, or more (e.g., all) of the following: (a) Col1a1; (b) FGF-21; (c) hydroxyproline content; (d) IL-1β; (e) matrix metalloproteinase (MMP), e.g., MMP-13, MMP-2, MMP-9, MT1-MMP, MMP-3, or MMP-10; (f) N-terminal fragment of type III collagen (proC3); (g) PIIINP (N-Terminal Propeptide of Type III Collagen); (h) α-smooth muscle actin (aSMA); (i) TGF-β; (j) tissue inhibitor of metalloproteinase (TIMP); e.g., TIMP1 or TIMP2; (k) Acta2; or (l) Hsp47.

8. The method of any of claim 1, 2, or 5-7, or composition for use of any of claims 3-7, wherein the total wt. % of (a)-(d) is greater than the total wt. % of one, two, or three of other amino acid entity components, non-amino acid entity protein components (e.g., whey protein), or non-protein components in the composition (e.g., in dry form).

9. The method of any of claim 1, 2, or 5-8, or composition for use of any of claims 3-8, wherein the composition comprises a combination of 18 or fewer amino acid entities.

10. The method of any of claim 1, 2, or 5-9, or composition for use of any of claims 3-9, wherein the composition does not comprise a peptide of more than 20 amino acid residues in length (e.g., whey protein), or if a peptide of more than 20 amino acid residues in length is present, the peptide is present at less than: 10 weight (wt.) % of the total wt. of protein components or total components the composition (e.g., in dry form).

11. The method of any of claim 1, 2, or 5-10, or composition for use of any of claims 3-10, wherein at least one, two, three, or four (e.g., all) of methionine (M), tryptophan (W), valine (V), or cysteine (C) is absent, or if present, are present at less than: 10 wt. % of the composition (e.g., in dry form).

12. The method of any of claim 1, 2, or 5-11, or composition for use of any of claims 3-11, wherein at least one, two, three, or more (e.g., all) of (a)-(d) is selected from Table 1.

13. The method of any of claim 1, 2, or 5-12, or composition for use of any of claims 3-12, wherein the composition further comprises one or both of (e) an isoleucine amino acid entity or (f) a valine amino acid entity.

14. The method of any of claim 1, 2, or 5-13, or composition for use of any of claims 3-13, wherein the wt. ratio of the leucine amino acid entity, the arginine amino acid entity, the glutamine amino acid entity, and the NAC-amino acid entity is 1+/−20%:1.5+/−20%:2+/−20%:0.15+/−20%.

15. The method of claim 13 or 14, or composition for use of claim 13 or 14, wherein the wt. ratio of the leucine amino acid entity, the isoleucine amino acid entity, the valine amino acid entity, the arginine amino acid entity, the glutamine amino acid entity, and the NAC-amino acid entity is 1+/−20%:0.5+/−20%:0.5+/−20%:1.5+/−20%:2+/−20%:0.15+/−20%.

16. The method of any of claim 1, 2, or 5-15, or composition for use of any of claims 3-15, wherein the composition comprises:
a) an leucine amino acid entity chosen from:
i) L-leucine or a salt thereof,
ii) a dipeptide or salt thereof, or tripeptide or salt thereof, comprising L-leucine, or
iii) β-hydroxy-β-methylbutyrate (HMB) or a salt thereof;
b) an arginine amino acid entity chosen from:
i) L-arginine or a salt thereof,
ii) a dipeptide or salt thereof, or tripeptide or salt thereof, comprising L-arginine,
iii) creatine or a salt thereof, or
iv) a dipeptide or salt thereof, or tripeptide or salt thereof, comprising creatine;
c) the glutamine amino acid entity is L-glutamine or a salt thereof or a dipeptide or salt thereof, or tripeptide or salt thereof, comprising L-glutamine; and
d) the NAC entity is NAC or a salt thereof or a dipeptide or salt thereof, comprising NAC.

17. The method of any of claim 1, 2, or 5-16, or composition for use of any of claims 3-16, wherein the composition further comprises one or both of:
e) L-isoleucine or a salt thereof or a dipeptide or salt thereof, or tripeptide or salt thereof, comprising L-isoleucine; or
f) L-valine or a salt thereof or a dipeptide or salt thereof, or tripeptide or salt thereof, comprising L-valine.

18. The method of any of claim 1, 2, or 5-17, or composition for use of any of claims 3-17, wherein the composition comprises:
a) the leucine amino acid entity is L-leucine or a salt thereof;
b) the arginine amino acid entity is L-arginine or a salt thereof;
c) the glutamine amino acid entity is L-glutamine or a salt thereof; and
d) the NAC entity is NAC or a salt thereof.

19. The method of any of claim 1, 2, or 5-18, or composition for use of any of claims 3-18, wherein the composition comprises:
a) the leucine amino acid entity is L-leucine or a salt thereof;
b) the arginine amino acid entity is L-arginine or a salt thereof;
c) the glutamine amino acid entity is L-glutamine or a salt thereof;
d) the NAC entity is NAC or a salt thereof;
e) the isoleucine amino acid entity is L-isoleucine or a salt thereof; and
f) the valine amino acid entity is L-valine or a salt thereof.

20. The method of any of claim 1, 2, or 5-19, or composition for use of any of claims 3-19, wherein the composition is formulated with a pharmaceutically acceptable carrier.

21. The method of any of claim 1, 2, or 5-20, or composition for use of any of claims 3-20, wherein the composition is formulated as a dietary composition.

22. The method of claim 21, or composition for use of claim 21, wherein the dietary composition is chosen from a medical food, a functional food, or a supplement.
